(19) United States
(12) Patent Application Publication
(10) Pub. No.: US 2024/0383980 A1
(43) Pub. Date: Nov. 21, 2024

(54) CD3/CD25 Antibodies For Neuro-Immune Diseases

(71) Applicant: Vitruviae LLC, Nutley, NJ (US)

(72) Inventors: Mahiuddin Ahmed, Verona, NJ (US); Sonia Sequeira, Verona, NJ (US)

(21) Appl. No.: 18/042,144

(22) PCT Filed: Aug. 19, 2021
(86) PCT No.: PCT/US2021/046674

§ 371 Date: Feb. 17, 2023

Related U.S. Application Data
(63) Continuation of application No. 16/997,570, filed on Aug. 19, 2020, now Pat. No. 11,124,568.

(51) Int. Cl. C07K 16/28

(52) U.S. Cl. CPC C07K 16/2809; C07K 16/2866; C07K 2317/24; C07K 2317/31; C07K 2317/52; C07K 2317/56; C07K 2317/565; C07K 2317/622

(57) ABSTRACT

The present invention relates to the use of anti-CD3 and anti-CD25 antibodies for the management and treatment of inflammatory diseases and psycho-immune disorders, such as depression, autism (ASD) and attention deficit/hyperactivity disorder (ADHD). In one aspect, the invention concerns an antibody or fragment thereof, capable of binding to CD3 and/or CD25. In another aspect, the invention concerns a pharmaceutical composition comprising an antibody or fragment thereof according to the invention and an excipient, such as a pharmaceutically acceptable carrier. Other aspects of the invention are also disclosed.

The present specification comprises a sequence listing in computer readable format, submitted together with the application. The sequence listing forms part of the disclosure and is incorporated in the specification in its entirety.

The present invention relates to the use of anti-CD3 and anti-CD25 antibodies for the management and treatment of inflammatory diseases and psycho-immune disorders, such as depression, autism (ASD) and attention deficit/hyperactivity disorder (ADHD).

Provided are compositions related to novel, humanized antibodies or bispecific antibodies, multivalent formats and adjuvants, carriers and methods of administration of such antibodies or bispecific antibodies, including by oral or nasal delivery in humans or preclinical models. This invention further relates to a companion diagnostic as a method of selection of subjects that may benefit from anti-CD3/anti-CD25 therapy and a biomarker for rapid and easy monitoring of response to treatment.

### TECHNICAL BACKGROUND

Inflammation is an immune response to protect the body from physical and emotional stressors that can cause or exacerbate psychological disorders (Miller and Raison 2016, Wang 2018, Leffa 2018, Dunn 2019, Gupta 2014, Siniscalco 2018).

Depression is currently the leading cause of disability globally (World Health Organization (WHO)—Depression and Other Common Mental Disorders Global Health Estimates, report 2017; NIH-NIMH, report 2017; BCBS report 2018; Gautam, 2017, www_who.int/news-room/fact-sheets/detail/depression, GBD 2018). The prevalence of depression ranges from 3% of the population in Japan to 17% in the United States (Orsolini 2020). In the United States, 3.2% of children ages 3-17 years (approximately 1.9 million) have diagnosed depression, frequently co-diagnosed with ADHD (17%) or ASD (14%) (CDC website, 2020, Ghandour 2018). Numbers have risen sharply in recent years, particularly among millennials and adolescents (47% for boys and 65% for girls since 2013). 50% of all new cases of depression are diagnosed before the age of 14 and 75% of cases by the age of 24 (BCBS report, 2018). Two thirds of youth suicide cases in the United States have been linked to depression and the overall risk for suicide for people with depression is estimated at 15% (Orsolini 2020, CDC, 2008).

### MECHANISMS OF DISEASE

Gut microbiota dysbiosis, which causes local, and through disruption of the intestinal barrier systemic inflammation (Belizario 2018) is prevalent in people with depression, ADHD and ASD (Valles-Colomer 2019, Zheng 2019, Bezewada 2020, Xu 2019, Stevens 2019), and may affect brain function through both systemic and autonomic pathways (Tengeler 2020, Sharon 2019).

Serotonin is an essential neurotransmitter that enhances mood, social connections, memory and healthy sleep patterns. Depression has been associated with a disturbance in serotonin (5-HT) and noradrenaline neurotransmission (Coopen and Swede, 1988) and MRI studies have suggested a correlation between increased risk of suicide and low levels of brain serotonin (Mann, 1990, Sullivan, 2015).

The relationship between inflammation and psychiatric disorders is not fully understood (Miller and Raison, 2016, Dantzer, 2011, Smith 1991, Ellul et al 2018). Smith (1991) suggested that depression is caused by excessive levels of pro-inflammatory cytokines in response to acute or chronic stress that penetrate the blood-brain barrier and alter the metabolism and synaptic signaling of neurotransmitters. Furthermore, brain microglia, activated by peripheral signals may release additional inflammatory cytokines locally.

It has been reported that brain areas targeted by cytokines overlap with those that are altered in depressed patients and include the prefrontal cortex (Juengling et al 2000), the anterior cingulate cortex (Capuron 2005), the putamen, the basal ganglia and cerebellum (Capuron et al 2007).

TREGs secrete IL-10 and TGF-b which inhibit cytotoxic T cells and their secretion of pro-inflammatory cytokines, preventing excessive activation of effector immune cells (Montufar Solis, 2007). Tregs express several surface molecules such as CTLA4, neuropilin-1 and LAG3 that modulate dendritic cells (DCs) to an immunosuppressive phenotype and further promote Treg cell expansion and function in a positive feedback mechanism.

Metabolic syndrome in young populations is associated with high levels of leptin, a a potent antagonist of adipose tissue resident Tregs, and treatment resistant depression (Snijders et al, 2016, Milaneschi, 2017).

### CD3 and CD25

CD3 is a protein complex and T cell co-receptor that is involved in activating T cells. CD3 is selectively expressed on T cells in blood, bone marrow and lymphoid tissues, but not on other normal tissues and with no cross reactivity to other animals except for chimpanzee.

CD25 (IL-2 receptor subunit a) is another T cell surface glycoprotein, expressed in activated T cells and present in the spleen, tonsils, marrow and lymphoid tissue. The anti-CD3 antibody Muromonab-CD3 and the anti-CD25 antibody Basiliximab are intravenous immunosuppressing treatments indicated for reversal and prevention of transplant rejection respectively.

IL-2 blocking anti-CD25 antibodies have been shown to ameliorate autoimmune and inflammatory conditions in human through inhibition of cytotoxic T cell proliferation and stimulation of TREGs which secrete the anti-inflammatory cytokines IL-10, IL-35 and TGF-B and further inhibit effector T cells.

### SUMMARY OF THE INVENTION

It has been estimated that 76% and 85% of people in low- and middle-income countries receive no treatment for their mental disorder due to the lack of mental health policies, financial aid, infrastructure and effective interventions (WHO, 2017; Wang, The Lancet, 2007). Thus, there is a need for easily available and effective means for management and treatment of such disorders.

The WHO has warned that current healthcare systems are not prepared to address the anticipated surge in mental disease burden in the next decade and that treatment options that are effective and safe are sorely needed (World Health Organization (WHO 2012, 2017). Thus, there is a need for effective and safe means for management and treatment of such disorders.

The lack of clear understanding of the etiology of depression, the lack of experimental models of disease and the lack of biomarkers to select patients that may benefit from a given treatment or to assess response as well as the prohibitive cost of currently available imaging technologies contribute to the challenges in developing therapies for depression (Arnow, 2015). Numerous clinical trials have failed and only a few new antidepressants have been approved in the last 20 years (Blackburn 2019). Thus, there is a need for effective and reliable means for diagnosing, monitoring and treating such disorders.

Currently available drugs for depression, ADHD or ASD are not optimized for children, have suboptimal efficacy, high levels of relapse/recurrence and associated with withdrawal syndrome or side-effects including black box warnings. Thus, there is a need for effective and safe means for management and treatment of such disorders in children and adolescents.

As many as ⅓ of the population diagnosed with any type of depression fails conventional therapy (Blackburn 2019), presenting recurrent or relapsed disease. Most of these individuals present inflammatory disease, in particular children previously exposed to adverse events (Blackburn 2019, Al-Harbi 2012, Dantzer 2011, Miller and Cole 2012). Similar findings linking inflammation to ADHD and ASD have been reported (Leffa 2018, Siniscalco 2019). Thus, there is a need for effective and safe means for management and treatment of such recurrent or relapsed disease.

To the best of the inventor's knowledge, there are no biological therapies in development for depression, ADHD or ASD, although the use of antibodies for the treatment of cancers and other inflammatory conditions is a well-known strategy (Labrijn 2019, Kaplon 2020, Lu 2020). Thus, there is a need for biological therapies for management and treatment of conditions such as depression, ADHD or ASD.

Standard of care antidepressants include selective serotonin reuptake inhibitors (SSRIs) but also tricyclic antidepressants, mirtazapine, bupropion, and venlafaxine, which may be taken indefinitely, until relapse or side-effects are observed. Optional treatments include electroconvulsive therapy (ECT) and psychosocial interventions, repetitive transcranial magnetic stimulation (rTMS), light therapy, transcranial direct stimulation, vagal nerve stimulation, deep brain stimulation and sleep deprivation treatment. Benzodiazepines may be used as adjunctive treatment, and lithium and thyroid supplements may be used as an augmenting agent when a patient is not responding to antidepressants (Gautam, 2017).

Studies have also shown that increased glutamate excitatory transmission predicts suicidal behavior in patients with MDD prompting the use of NMDA glutamate receptor inhibitors ketamine or esketamine (Yuksel and Orgun, 2010, Serafini, 2015, Matthews, 2012, Zhao, 2018, Lent 2019, esketamine FDA label).

Despite the available drug repertoire, as many as ⅓ of the population diagnosed with any type of depression fails conventional therapy (Al-Harbi, 2012), presenting resistant or relapsed disease. Thus, there is a need for effective and safe means for management and treatment of depression. There is a need for effective and safe means for management and treatment of depression in subjects that fails conventional therapy. There is a need for effective and safe means for management and treatment of resistant or relapsed depression.

In addition, current medication for ADHD and ASD is based on drugs with substantial side-effects. Methylphenidate and atomoxetine inhibit the re-uptake of dopamine and norepinephrine respectively and are approved for ADHD. Risperidone and aripiprazole are non-indication specific anti-psychotics and the only drugs approved by the FDA for children with autism spectrum disorder to help with irritability. Thus, there is a need for means of management and treatment of such disorders with fewer side-effects and potentially more suitable for children and adolescents.

A wide range of anti-inflammatory agents such as COX2 inhibitors have been tested in patients suffering from major depressive disorder however their efficacy requires further evaluation (MDD) (Muller, 2010). Thus, there is a need for effective and safe means for management and treatment of such disorders.

Several non-clinical and clinical studies suggest that inflammation is related at least in part by regulatory T cells (TREGs) insufficiency (da Cunha, 2011, Kuhn and Weiner, 2016, Moaaz, 2019) which inhabit the intestinal tract. Moaaz et al 2019 showed a systemic TREG imbalance that correlated negatively with the severity of disease in children with ASD. The role of TREGs has been also described in Inflammatory Bowel Disease (Ding 2020). Low dose IL-2 has been used clinically to stimulate TREGs in patients with hepatitis C virus-induced Vasculitis, chronic graft vs host disease, alopecia, systemic lupus erythematosus and other autoimmune disorders (Ellul 2018, clinical trial NCT01988506). In mice, intraperitoneal administration of anti-CD25 antibodies to deplete CD25+ TREGs resulted in increased despair behavior (Kim et al 2012).

Thus, there is a need for a better understanding of TREGs role in these diseases.

Recently, human CD3 transgenic mice have been engineered, facilitating the study of anti-CD3 immunotherapies. Anti-CD3 based therapies such as muromomab-CD3 (Janssen, Orthoclone, OKT3) have been extensively studied in humans both systemically and orally for ulcerative colitis and metabolic syndrome (da Cunha 2011, Ilan 2010—NCT01287195, NCT01205087). Thus, there is a need for improved therapeutics targeting CD3.

Anti-CD3 bispecific antibody platforms that bridge tumors and T cells such as blinatumomab and catumaxomab have been approved for the treatment of cancer and several other CD3 bispecifics are in clinical development (Suurs, 2019).

Systemic anti-CD3 therapy for management of graft rejection is associated with high toxicity due to general depletion of T cells and cytokine release syndrome (Kuhn and Weiner, 2016, X). Moreover, early studies used murine antibodies, limiting their clinical utility, and precluding high or repeat dosing strategies. Thus, there is a need for improved therapeutics targeting CD3.

Da Cunha (2011), Ochi (2006), Kuhn (2016), Ishikawa (2007), Boden (2019) and Rezende (2019) have described the immunosuppressive effect of murine and humanized anti-CD3 antibodies (moromomab, teplizumab, visilizumab and foralumab) in autoimmune diseases, hepatitis and diabetes.

Daclixumab was approved for multiple sclerosis and was later withdrawn due to safety concerns. Several groups are currently investigating the use of anti-CD25 antibodies to target cancer (camidanlumab-Genmab, αCD25NIB-Roche, AACR 2018 abstract 2787 and 192). Thus, there is a need for improved therapeutics targeting CD25

It is speculated that a CD3/CD25 multivalent antibody format will minimize off-target, non-TREG T cell binding and increase the potency of an anti-inflammatory, anti-depressive effect. TREG activation inhibits effector T cell pro-inflammatory cytokine release, secretes immune suppressive cytokines, and activates dendritic cells to produce substrates necessary for neurotransmitter synthesis such as tryptophan for serotonin (5H-T) and inhibit NMDA mediated excitatory signaling.

According to an aspect, the invention concerns an antibody or fragment thereof, capable of binding to CD3 and/or CD25.

According to another aspect, the invention concerns a pharmaceutical composition comprising an antibody or fragment thereof according to the invention and an excipient, such as a pharmaceutically acceptable carrier.

According to another aspect, the invention concerns a method of preventing, treating and/or alleviating an inflammatory disease, a psycho-immune disorder, an auto-immune disease and/or a mood disorder comprising administering to a patient in need thereof a therapeutically effective amount of the antibody or fragment thereof and/or pharmaceutical composition according to the invention.

According to another aspect, the invention concerns a method of preventing, treating and/or alleviating an inflammatory disease comprising administering to a patient in need thereof a therapeutically effective amount of the antibody or fragment thereof and/or pharmaceutical composition according to the invention.

According to another aspect, the invention concerns a method of preventing, treating and/or alleviating a mood disorder comprising administering to a patient in need thereof a therapeutically effective amount of the antibody or fragment thereof and/or pharmaceutical composition according to the invention.

According to another aspect, the invention concerns a method of preventing, treating and/or alleviating a psycho-immune disorder comprising administering to a patient in need thereof a therapeutically effective amount of the antibody or fragment thereof and/or pharmaceutical composition according to the invention.

According to another aspect, the invention concerns a diagnostic kit comprising the antibody or fragment thereof according to the invention and instructions for use.

According to another aspect, the invention concerns a method of diagnosing a disease in a subject, wherein said method comprises the following steps:
a) Providing a blood, nasal and/or salivary sample from said subject.
b) Contacting said blood, nasal and/or salivary sample with an antibody or fragment thereof according to the invention.

According to another aspect, the invention concerns a method of screening and/or monitoring progression of a disease in a subject, wherein said method comprises the following steps:
a) Providing a blood, nasal and/or salivary sample from said subject.
b) Contacting said blood, nasal and/or salivary sample with an antibody or fragment thereof according to the invention.

According to another aspect, the invention concerns an isolated nucleic acid molecule encoding an antibody agent or fragment thereof according to the invention.

According to another aspect, the invention concerns a recombinant vector comprising a nucleic acid molecule of the invention.

According to another aspect, the invention concerns a host cell comprising the recombinant vector of the invention.

According to another aspect, the invention concerns a method to produce an antibody or fragment thereof according to the invention comprising a step of culturing the host cell according to the invention in a culture medium under conditions allowing the expression of the antibody or fragment thereof and separating the antibody or fragment thereof from the culture medium.

According to another aspect, the invention concerns a method for generating a heterodimeric antibody, said method comprising the following steps:
a) providing a first homodimeric antibody comprising a first Fc region, said first Fc region comprising a first CH3 region selected from the group consisting of a human IgG2 CH3 region and a human IgG4 CH3 region, but with an amino acid substitution at a position selected from the group consisting of: 405, 409, 234, 235 and 322 (EU numbering);
b) providing a second homodimeric antibody comprising a second Fc region, said second Fc region comprising a second CH3 region selected from the group consisting of a human IgG2 CH3 region and a human IgG4 CH3 region, but with an amino acid substitution at a position selected from the group consisting of: 405, 409, 234, 235 and 322 (EU numbering),
   wherein the sequences of said first and second antibody CH3 regions are different,
c) incubating said first antibody together with said second antibody under reducing conditions sufficient to allow the cysteines in the hinge region to undergo disulfide-bond isomerization,
   to obtain a heterodimeric antibody.

According to another aspect, the invention concerns a method for generating a heterodimeric antibody, said method comprising the following steps:
a) providing a first homodimeric antibody comprising an Fc region, said Fc region comprising a first CH3 region selected from the group consisting of a human IgG2 CH3 region and a human IgG4 CH3 region, but with an amino acid substitution at position 409 (EU numbering);
b) providing a second homodimeric antibody comprising an Fc region, said Fc region comprising a second CH3 region selected from the group consisting of a human IgG2 CH3 region and a human IgG4 CH3 region, but with an amino acid substitution at position 405 (EU numbering),
c) incubating said first antibody together with said second antibody under reducing conditions sufficient to allow the cysteines in the hinge region to undergo disulfide-bond isomerization,
   to obtain a heterodimeric antibody,
   wherein the heterodimeric interaction between said first and second antibodies in the resulting heterodimeric antibody is such that no Fab-arm exchange occurs at 0.5 mM GSH after 24 hours at 37° C.

According to another aspect, the invention concerns a method for generating a heterodimeric antibody, said method comprising the following steps:
a) providing a first homodimeric antibody comprising an Fc region, said Fc region comprising a first CH3 region selected from the group consisting of a human IgG2 CH3 region and a human IgG4 CH3 region;
b) providing a second homodimeric antibody comprising an Fc region, said Fc region comprising a second CH3 region selected from the group consisting of a human IgG2 CH3 region and a human IgG4 CH3 region, but with an amino acid substitution at position 405 and an amino acid substitution at position 409 (EU numbering),
c) incubating said first antibody together with said second antibody under reducing conditions sufficient to allow the cysteines in the hinge region to undergo disulfide-bond isomerization,
   to obtain a heterodimeric antibody,
   wherein the heterodimeric interaction between said first and second antibodies in the resulting heterodimeric antibody is such that no Fab-arm exchange occurs at 0.5 mM GSH after 24 hours at 37° C.

According to another aspect, the invention concerns a heterodimeric antibody obtainable by the method according to the invention.

According to another aspect, the invention concerns a heterodimeric antibody comprising:

A first heavy chain comprising a first Fc region, said first Fc region comprising a first CH3 region selected from the group consisting of a human IgG2 CH3 region and a human IgG4 CH3 region, but with an amino acid substitution at position 405 (EU numbering), and

A second heavy chain comprising a second Fc region, said second Fc region comprising a second CH3 region selected from the group consisting of a human IgG2 CH3 region and a human IgG4 CH3, but with an amino acid substitution at position 409 (EU numbering);

wherein the sequences of the first and second CH3 regions are different.

According to another aspect, the invention concerns a heterodimeric antibody comprising:

A first heavy chain comprising a first Fc region, said first Fc region comprising a first CH3 region selected from the group consisting of a human IgG2 CH3 region and a human IgG4 CH3 region, and

A second heavy chain comprising a second Fc region, said second Fc region comprising a second CH3 region selected from the group consisting of a human IgG2 CH3 region and a human IgG4 CH3, but with an amino acid substitution at position 405 and an amino acid substitution at position 409 (EU numbering);

wherein the sequences of the first and second CH3 regions are different.

According to another aspect, the invention concerns a pharmaceutical composition comprising a heterodimeric antibody according to the invention and an excipient, such as a pharmaceutical carrier.

According to another aspect, the invention concerns a bispecific antibody or fragment thereof comprising a first antigen binding site capable of binding to a first antigen and a second antigen binding site capable of binding to a second antigen, wherein said first antigen binding site is comprised in a Fab fragment and said second antigen binding site is comprised in a moiety selected from the group consisting of scFv, antibody fragments and protein moiety, wherein said moiety being attached to the light chain of said Fab fragment.

According to another aspect, the invention concerns a heterodimeric antibody, wherein said heterodimeric antibody comprises a human IgG4 Fc domain, wherein said heterodimeric antibody binds to two or more different targets and wherein said heterodimeric antibody further comprises a component selected from
(i) a Fab,
(ii) a scFv,
(iii) a nanobody,
(iv) a variable heavy homodimer (VHH), and/or
(v) an antibody fragment.

According to another aspect, the invention concerns a heterodimeric antibody, wherein said heterodimeric antibody comprises a human IgG2 Fc domain, wherein said heterodimeric antibody binds to two or more different targets and wherein said heterodimeric antibody further comprises a component selected from
(i) a Fab,
(ii) a scFv,
(iii) a nanobody,
(iv) a variable heavy homodimer (VHH), and/or
(v) an antibody fragment.

According to another aspect, the invention concerns an IgG2, IgG3 or IgG4 molecule that comprises a light chain and an antibody domain and/or protein fused to the C-terminus of said light chain, wherein said antibody domain is selected from
(i) a Fab,
(ii) a scFv,
(iii) a nanobody,
(iv) a variable heavy homodimer (VHH), and/or
(v) an antibody fragment.

### DETAILED DISCLOSURE

According to embodiments of the invention it relates to antibodies.

According to an embodiment, the invention relates to antibodies or fragments thereof capable of binding to CD3 and/or CD25. The antibodies may be natural proteins, i.e. proteins isolated from a natural host organism or proteins having an identical amino acid sequence to such a protein; or it may be artificial proteins i.e. proteins that have been designed and/or constructed artificially, and wherein a corresponding protein having identical amino acid sequence have not been isolated from a natural host organism.

The binding site capable of binding to CD3 or CD25 may be an ordinary antibody binding site comprising a first polypeptide chain comprising a VH sequence and a second polypeptide chain comprising a VL sequence, a scFv site containing a single polypeptide containing VH and/or VL sequences, or it may be a nanobody comprising the binding site derived from a single chain antibody, as know from e.g. camelids, such as camels and alpacas.

Examples of artificial proteins include humanized antibodies, where a non-human antibody is altered by substituting one or more amino acid residues, usually outside the CDR sequences, with sequences found in human antibodies, to alter the complete antibody into an antibody that is more “human-like” than the original non-human antibody (also known as “humanized”). Other examples of artificial proteins include antibody fragments, in particular antibody fragments capable of binding to CD3 and/or CD25, scFv fragments and nanobodies.

The antibodies may be monomeric or multimeric, such as dimeric or tetrameric. Dimeric antibodies may be homomeric, when the molecule comprises two identical binding sites or it may be heteromeric when the molecule comprises two different binding sites.

According to an embodiment, the invention concerns an antibody or fragment thereof, capable of binding to CD3 and/or CD25.

In one embodiment the antibody of the invention comprises two identical polypeptides, each comprising Fc sequences, a first scFv capable of binding CD3 and/or a second scFv capable of binding CD25.

According to an embodiment, the invention concerns the antibody or fragment thereof, capable of binding to CD3.

According to an embodiment, the invention concerns the antibody or fragment thereof, wherein said antibody or fragment thereof comprises at least one of the amino acid sequences of CDR1, CDR2 and CDR3 of the murine antibody OKT3 variable light chain and/or at least one of the amino acid sequences of CDR1, CDR2 and CDR3 of the murine antibody OKT3 variable heavy chain.

According to an embodiment, the invention concerns the antibody or fragment thereof, wherein said antibody or fragment thereof comprises the amino acid sequences of CDR1, CDR2 and CDR3 of the murine antibody OKT3 variable light chain and/or the amino acid sequences of CDR1, CDR2 and CDR3 of the murine antibody OKT3 variable heavy chain.

According to an embodiment, the invention concerns the antibody or fragment thereof, wherein said antibody or fragment thereof comprises variable light chain CDR sequences of SEQ ID NO:73-75.

According to an embodiment, the invention concerns the antibody or fragment thereof, wherein said antibody or fragment thereof comprises variable heavy chain CDR sequences of SEQ ID NO:76-78.

According to an embodiment, the invention concerns the antibody or fragment thereof, wherein said antibody or fragment thereof comprises variable light chain CDR sequences of SEQ ID NO:73-75 and variable heavy chain CDR sequences of SEQ ID NO: 76-78.

According to an embodiment, the invention concerns the antibody or fragment thereof, wherein all antigen contact residues of murine antibody OKT3 are contained.

According to an embodiment, the invention concerns the antibody or fragment thereof, wherein all CDR sequences of murine antibody OKT3 are contained.

According to an embodiment, the invention concerns the antibody or fragment thereof wherein all antigen contact residues and all CDR sequences of murine antibody OKT3 are contained.

According to an embodiment, the invention concerns the antibody or fragment thereof comprising the murine antibody OKT3 variable heavy chain residue K82 (IMGT numbering).

According to an embodiment, the invention concerns the antibody or fragment thereof, wherein said antibody or fragment thereof comprises a variable heavy chain domain of SEQ ID NO:6-10 and a variable light chain domain of SEQ ID NO:1-5.

According to an embodiment, the invention concerns the antibody or fragment thereof, wherein said antibody or fragment thereof comprises a Cys114Ser mutation (IMGT numbering) in the heavy chain CDR3 region.

According to an embodiment, the invention concerns a humanized antibody of fragments thereof capable of binding CD3, based on the alpaca VHH nanobody F10.

According to an embodiment the humanized antibody of fragments thereof capable of binding CD3 comprises CDR sequences of SEQ ID NO: 131, SEQ ID NO: 132 and SEQ ID NO: 133, or CDR sequences that differed from one of these sequences by 1 substitution.

According to an embodiment, the humanized antibody of fragments thereof capable of binding CD3, based on the alpaca VHH nanobody F10, are selected among antibodies comprising the sequences of SEQ ID NO: 123 or SEQ ID NO: 124.

According to an embodiment, the invention concerns the antibody or fragment thereof, capable of binding to CD25.

According to an embodiment, the invention concerns the antibody or fragment thereof, wherein said antibody or fragment thereof comprises at least one of the amino acid sequences of CDR1, CDR2 and CDR3 of the monoclonal antibody Basiliximab variable light chain and/or at least one of the amino acid sequences of CDR1, CDR2 and CDR3 of the monoclonal antibody Basiliximab variable heavy chain.

According to an embodiment, the invention concerns the antibody or fragment thereof, wherein said antibody or fragment thereof comprises the amino acid sequences of CDR1, CDR2 and CDR3 of the monoclonal antibody Basiliximab variable light chain and/or the amino acid sequences of CDR1, CDR2 and CDR3 of the monoclonal antibody Basiliximab variable heavy chain.

According to an embodiment, the invention concerns the antibody or fragment thereof, wherein said antibody or fragment thereof comprises variable light chain CDR sequences of SEQ ID NO:79-81.

According to an embodiment, the invention concerns the antibody or fragment thereof, wherein said antibody or fragment thereof comprises variable heavy chain CDR sequences of SEQ ID NO:82-84.

According to an embodiment, the invention concerns the antibody or fragment thereof, wherein said antibody or fragment thereof comprises variable light chain CDR sequences of SEQ ID NO:79-81 and variable heavy chain CDR sequences of SEQ ID NO:82-84.

According to an embodiment, the invention concerns the antibody or fragment thereof, wherein all antigen contact residues of monoclonal antibody Basiliximab are contained.

According to an embodiment, the invention concerns the antibody or fragment thereof, wherein all CDR sequences of monoclonal antibody Basiliximab are contained.

According to an embodiment, the invention concerns the antibody or fragment thereof, wherein all antigen contact residues and all CDR sequences of monoclonal antibody Basiliximab are contained.

According to an embodiment, the invention concerns the antibody or fragment thereof, wherein said antibody or fragment thereof comprises a variable heavy chain domain of SEQ ID NO:29-31 and a variable light chain domain of SEQ ID NO:32-34.

According to an embodiment, the invention concerns the antibody or fragment thereof, wherein said antibody or fragment thereof is humanized.

According to an embodiment, the invention concerns the antibody or fragment thereof, wherein said antibody or fragment thereof is humanized and comprises at least one of the amino acid sequences of CDR1, CDR2 and CDR3 of the monoclonal antibody 7G7 variable light chain and/or at least one of the amino acid sequences of CDR1, CDR2 and CDR3 of the monoclonal antibody 7G7 variable heavy chain.

According to an embodiment, the CDR1, CDR2 and CDR3 of the monoclonal antibody 7G7 variable light chain have the sequences of SEQ ID NO: 125, SEQ ID NO: 126 and SEQ ID NO: 127.

According to an embodiment, the CDR1, CDR2 and CDR3 of the monoclonal antibody 7G7 variable heavy chain have the sequences of SEQ ID NO: 128, SEQ ID NO: 129 and SEQ ID NO: 130.

According to an embodiment, the invention concerns a humanized antibody or a fragment thereof comprising at least one of the amino acid sequences of CDR1, CDR2 and CDR3 of the monoclonal antibody 7G7 variable light chain and/or at least one of the amino acid sequences of CDR1, CDR2 and CDR3 of the monoclonal antibody 7G7 variable heavy chain, selected among antibodies comprising the VL sequence disclosed as SEQ ID NO: 118, and the VH sequence of SEQ ID NO: 119, or comprises the VL sequence of SEQ ID NO: 120 and the VH sequence of SEQ ID NO: 121.

According to an embodiment, the invention concerns the antibody or fragment thereof, wherein said antibody or fragment thereof comprises a scFv.

According to an embodiment, the invention concerns the antibody or fragment thereof, wherein said antibody or fragment thereof is a scFv.

According to an embodiment, the invention concerns the antibody or fragment thereof, wherein said scFv has increased disulfide stabilization.

According to an embodiment, the invention concerns the antibody or fragment thereof, wherein said scFv comprises at least one Cys substitution at a position selected from VH44-VL100 according to Kabat numbering.

According to an embodiment, the invention concerns the antibody or fragment thereof, wherein said antibody or fragment thereof comprises a linker.

According to an embodiment, the invention concerns the antibody or fragment thereof, wherein said scFv comprises a linker.

According to an embodiment, the invention concerns the antibody or fragment thereof, wherein said linker is a peptide linker.

According to an embodiment, the invention concerns the antibody or fragment thereof, wherein said linker comprises the sequence of SEQ ID NO:71.

According to an embodiment, the invention concerns the antibody or fragment thereof, wherein said scFv binds CD3 and comprises a sequence selected from the group consisting of SEQ ID NO:11-28.

According to an embodiment, the invention concerns the antibody or fragment thereof, wherein said scFv binds CD25 and comprises a sequence selected from the group consisting of SEQ ID NO:35-50.

According to an embodiment, the invention concerns the antibody or fragment thereof, wherein said antibody or fragment thereof comprises a constant light chain (CL) domain selected from the group consisting of kappa CL domain and lambda CL domain.

According to an embodiment, the invention concerns the antibody or fragment thereof, wherein said antibody or fragment thereof comprises a sequence selected from the group consisting of a kappa CL domain sequence and a lambda CL domain sequence.

According to an embodiment, the invention concerns the antibody or fragment thereof, wherein said antibody or fragment thereof comprises a sequence of SEQ ID NO:70.

According to an embodiment, the invention concerns the antibody or fragment thereof, wherein said antibody or fragment thereof is a heterodimeric construct.

According to an embodiment, the invention concerns the antibody or fragment thereof, wherein said heterodimeric construct comprises IgG2 and/or IgG4 constant domain.

According to an embodiment, the invention concerns the antibody or fragment thereof, wherein said heterodimeric construct comprises a point mutation selected from the group consisting of K409R, R409K, F405L, L234A, F234A, V234A, L235A, K322A and S228P.

According to an embodiment, the invention concerns the antibody or fragment thereof, wherein said antibody or fragment thereof comprises a sequence selected from the group consisting of SEQ ID NO:51-69.

According to an embodiment, the invention concerns the antibody or fragment thereof, wherein said antibody or fragment thereof is a monospecific antibody.

According to an embodiment, the invention concerns the antibody or fragment thereof, wherein said antibody or fragment thereof is a bispecific antibody.

According to an embodiment, the invention concerns the antibody or fragment thereof, wherein said antibody or fragment thereof is a multispecific antibody.

According to an embodiment, the invention concerns the antibody or fragment thereof, wherein said antibody or fragment thereof is a 4 chain IgG antibody.

According to an embodiment, the invention concerns the antibody or fragment thereof, wherein said antibody or fragment thereof is a 2 chain scFv-Fc antibody.

According to an embodiment, the invention concerns the antibody or fragment thereof, wherein said antibody or fragment thereof is a 2 chain scFv-Fc-scFv antibody.

According to an embodiment, the invention concerns the antibody or fragment thereof, wherein said antibody or fragment thereof is 2 chain scFv-IgG4-Fc antibody.

According to an embodiment, the invention concerns the antibody or fragment thereof, wherein said antibody or fragment thereof comprises a first antigen binding site capable of binding to CD3 and a second antigen binding site capable of binding to CD25, wherein said first antigen binding site is comprised in a Fab fragment and said second antigen binding site is comprised in a moiety selected from the group consisting of scFv, antibody fragments and protein moiety, wherein said moiety being attached to the light chain of said Fab fragment.

According to an embodiment, the invention concerns the antibody or fragment thereof, wherein said moiety is a scFv.

According to an embodiment, the invention concerns the antibody or fragment thereof, wherein said moiety is attached to the C-terminus or N-terminus of the light chain of said Fab fragment.

According to an embodiment, the invention concerns the antibody or fragment thereof, wherein said moiety is attached to the C-terminus of the light chain of said Fab fragment.

According to an embodiment, the invention concerns the antibody or fragment thereof, wherein said Fab fragment is derived from an IgG or an IgM.

According to an embodiment, the invention concerns the antibody or fragment thereof, wherein said Fab fragment is derived from an IgG.

According to an embodiment, the invention concerns the antibody or fragment thereof, wherein said Fab fragment is derived from an IgG selected from the group consisting of IgG1, IgG2, IgG3 and IgG4.

According to an embodiment, the invention concerns the antibody or fragment thereof, wherein said Fab fragment is derived from an IgG2 or an IgG4.

According to an embodiment, the invention concerns the antibody or fragment thereof, wherein said antibody or fragment thereof is isolated.

According to an embodiment, the invention concerns the antibody or fragment thereof, wherein said antibody or fragment thereof has increased stability and/or increased manufacturability.

According to embodiments, the invention relates to pharmaceutical composition comprising antibodies.

According to an embodiment, the invention concerns a pharmaceutical composition comprising an antibody or fragment thereof according to the invention and an excipient, such as a pharmaceutically-acceptable carrier.

According to an embodiment, the invention concerns the pharmaceutical composition comprising an adjuvant.

According to an embodiment, the invention concerns the pharmaceutical composition, wherein said adjuvant is selected from the group consisting of β-D-galactosylceramide, α-D-galactosylceramide and β-glucan.

According to an embodiment, the invention concerns the pharmaceutical composition, wherein said pharmaceutical composition is a stabilized pharmaceutical.

According to an embodiment, the invention concerns the antibody or fragment thereof and/or pharmaceutical composition according to the invention, wherein said antibody or antigen binding fragment and/or pharmaceutical composition allows administration through a route selected among subcutaneous administration, intradermal administration, intramuscular administration, oral administration and/or nasal administration.

According to embodiments, the invention relates to a method of treatment using antibodies or a composition of the invention.

According to an embodiment, the invention concerns the antibody or fragment thereof and/or pharmaceutical composition according to the invention, wherein said antibody or antigen binding fragment and/or pharmaceutical composition allows administration through a route selected among oral and nasal administration.

According to an embodiment, the invention concerns a method of preventing, treating and/or alleviating an inflammatory disease, a psycho-immune disorder, an auto-immune disease and/or a mood disorder comprising administering to a patient in need thereof a therapeutically effective amount of the antibody or fragment thereof and/or pharmaceutical composition according to the invention.

According to an embodiment, the invention concerns a method of preventing, treating and/or alleviating an inflammatory disease comprising administering to a patient in need thereof a therapeutically effective amount of the antibody or fragment thereof and/or pharmaceutical composition according to the invention.

According to an embodiment, the invention concerns a method of preventing, treating and/or alleviating a mood disorder comprising administering to a patient in need thereof a therapeutically effective amount of the antibody or fragment thereof and/or pharmaceutical composition according to the invention.

According to an embodiment, the invention concerns a method of preventing, treating and/or alleviating a psycho-immune disorder comprising administering to a patient in need thereof a therapeutically effective amount of the antibody or fragment thereof and/or pharmaceutical composition according to the invention.

According to an embodiment, the invention concerns the method, wherein said inflammatory disease, psycho-immune disorder and/or a mood disorder is selected from the group consisting of depression, major depressive disorder, autism (ASD) and attention deficit/hyperactivity disorder (ADHD), bipolar disorder, seasonal affective disorder (SAD), cyclothymic disorder, premenstrual dysphoric disorder, persistent depressive disorder, disruptive mood dysregulation disorder, depression related to medical illness, depression induced by substance use or medication.

According to an embodiment, the invention concerns the method, wherein said subject is an adult or a pediatric subject.

According to an embodiment, the invention concerns the method, wherein said antibody or fragment thereof and/or pharmaceutical composition is administered subcutaneously, intradermally, intramuscularly, orally and/or nasally.

According to an embodiment, the invention concerns the method, wherein said antibody or fragment thereof and/or pharmaceutical composition is administered subcutaneously, intradermally, intramuscularly, orally and/or nasally.

According to embodiments, the invention relates to diagnostic methods using antibodies of the invention.

According to an embodiment, the invention concerns a diagnostic kit comprising the antibody or fragment thereof according to the invention and instructions for use.

According to an embodiment, the invention concerns the diagnostic kit, wherein said diagnostic kit is for companion diagnostic.

According to an embodiment, the invention concerns the diagnostic kit, wherein said diagnostic kit is for the selection of patients that may benefit from treatment with an antibody of fragment thereof according to the invention.

According to an embodiment, the invention concerns a method of diagnosing a disease in a subject, wherein said method comprises the following steps:
a. Providing a blood and/or salivary sample from said subject.
b. Contacting said blood and/or salivary sample with an antibody or fragment thereof according to the invention.

According to an embodiment, the invention concerns a method of screening and/or monitoring progression of a disease in a subject, wherein said method comprises the following steps:
a. Providing a blood and/or salivary sample from said subject.
b. Contacting said blood and/or salivary sample with an antibody or fragment thereof according to the invention.

According to an embodiment, the invention concerns the method, wherein blood and/or salivary sample is monitored for a blood and/or salivary biomarker.

According to an embodiment, the invention concerns the method, wherein blood and/or salivary biomarker is a TREG cell biomarker.

According to embodiments, the invention relates to nucleic acids encoding antibodies of the invention, vectors, expression constructs comprising said nucleic acids and method for producing antibodies of the invention.

According to an embodiment, the invention concerns an isolated nucleic acid molecule encoding an antibody agent or fragment thereof according to the invention.

According to an embodiment, the invention concerns a recombinant vector comprising the nucleic acid molecule of the invention.

According to an embodiment, the invention concerns a host cell comprising the recombinant vector of the invention.

According to an embodiment, the invention concerns a method for the production of an antibody or fragment thereof according to the invention comprising a step of culturing the host cell according to the invention in a culture medium under conditions allowing the expression of the antibody or fragment thereof and separating the antibody or fragment thereof from the culture medium.

According to an embodiment, the invention concerns the antibody or fragment thereof, wherein the antibody or fragment thereof is produced by a recombinant vector comprising a nucleic acid encoding said antibody.

According to an embodiment, the invention concerns a method for generating a heterodimeric antibody, said method comprising the following steps:
a) providing a first homodimeric antibody comprising a first Fc region, said first Fc region comprising a first CH3 region selected from the group consisting of a human IgG2 CH3 region and a human IgG4 CH3 region, but with an amino acid substitution at a position selected from the group consisting of: 405, 409, 234, 235 and 322 (EU numbering);
b) providing a second homodimeric antibody comprising a second Fc region, said second Fc region comprising a second CH3 region selected from the group consisting of a human IgG2 CH3 region and a human IgG4 CH3 region, but with an amino acid substitution at a position selected from the group consisting of: 405, 409, 234, 235 and 322 (EU numbering),
wherein the sequences of said first and second antibody CH3 regions are different,
c) incubating said first antibody together with said second antibody under reducing conditions sufficient to allow the cysteines in the hinge region to undergo disulfide-bond isomerization,
   to obtain a heterodimeric antibody.

According to an embodiment, the invention concerns a method for generating a heterodimeric antibody, said method comprising the following steps:
a) providing a first homodimeric antibody comprising an Fc region, said Fc region comprising a first CH3 region selected from the group consisting of a human IgG2 CH3 region and a human IgG4 CH3 region, but with an amino acid substitution at position 409 (EU numbering);
b) providing a second homodimeric antibody comprising an Fc region, said Fc region comprising a second CH3 region selected from the group consisting of a human IgG2 CH3 region and a human IgG4 CH3 region, but with an amino acid substitution at position 405 (EU numbering),
c) incubating said first antibody together with said second antibody under reducing conditions sufficient to allow the cysteines in the hinge region to undergo disulfide-bond isomerization,
   to obtain a heterodimeric antibody,
   wherein the heterodimeric interaction between said first and second antibodies in the resulting heterodimeric antibody is such that no Fab-arm exchange occurs at 0.5 mM GSH after 24 hours at 37° C.

According to an embodiment, the invention concerns a method for generating a heterodimeric antibody, said method comprising the following steps:
a) providing a first homodimeric antibody comprising an Fc region, said Fc region comprising a first CH3 region selected from the group consisting of a human IgG2 CH3 region and a human IgG4 CH3 region;
b) providing a second homodimeric antibody comprising an Fc region, said Fc region comprising a second CH3 region selected from the group consisting of a human IgG2 CH3 region and a human IgG4 CH3 region, but with an amino acid substitution at position 405 and an amino acid substitution at position 409 (EU numbering),
c) incubating said first antibody together with said second antibody under reducing conditions sufficient to allow the cysteines in the hinge region to undergo disulfide-bond isomerization,
   to obtain a heterodimeric antibody,
   wherein the heterodimeric interaction between said first and second antibodies in the resulting heterodimeric antibody is such that no Fab-arm exchange occurs at 0.5 mM GSH after 24 hours at 37° C.

According to an embodiment, the invention concerns the method, wherein said method is performed in vitro.

According to an embodiment, the invention concerns the method, wherein said first and/or second CH3 region is of human IgG2.

According to an embodiment, the invention concerns the method, wherein said first and/or second CH3 region is of human IgG4.

According to an embodiment, the invention concerns the method, wherein said first and/or second Fc region is of human IgG2 or human IgG4.

According to an embodiment, the invention concerns the method, wherein said heterodimeric antibody comprises a hinge region selected from a IgG1 hinge region or a IgG4 hinge region with a S228P mutation.

According to an embodiment, the invention concerns the method, wherein said first Fc region and/or said second Fc region is chimeric, humanized, or human.

According to an embodiment, the invention concerns the method, wherein said amino acid substitution is selected from the group consisting of: F405L, K409R, L234A, F234A, V234A, L235A, N297A and K322A (EU numbering).

According to an embodiment, the invention concerns the method, wherein said first and second homodimeric antibodies bind different epitopes.

According to an embodiment, the invention concerns the method, wherein the heterodimeric interaction between said first and second antibodies in the resulting heterodimeric antibody is such that no Fab-arm exchange occurs.

According to an embodiment, the invention concerns the method, wherein the heterodimeric interaction between said first and second antibodies in the resulting heterodimeric antibody is such that no Fab-arm exchange occurs at 0.5 mM GSH after 24 hours at 37° C.

According to an embodiment, the invention concerns the method, wherein the heterodimeric interaction between said first and second antibodies in the resulting heterodimeric antibody is such that no Fab-arm exchange occurs and wherein said first and/or second Fc region is of human IgG4 but with an amino acid substitution at a position 228 (EU numbering).

According to an embodiment, the invention concerns the method, wherein said amino acid substitution is S228P.

According to an embodiment, the invention concerns the method, wherein said first homodimeric antibody has an amino acid other than Lys at position 409 and said second homodimeric antibody has an amino acid other than Phe at position 405.

According to an embodiment, the invention concerns the method, wherein said first homodimeric antibody comprises an Arg at position 409 and said second homodimeric antibody comprises a Leu at position 405.

According to an embodiment, the invention concerns the method, wherein said first and second homodimeric antibodies provided in steps a) and b) are purified.

According to an embodiment, the invention concerns the method, wherein said first and/or second homodimeric antibody is conjugated to a drug, a prodrug or a toxin or contains an acceptor group for the same.

According to an embodiment, the invention concerns the method, wherein the reducing conditions in step c) comprise the addition of a reducing agent.

According to an embodiment, the invention concerns the method, wherein step d) comprises removal of a reducing agent.

According to an embodiment, the invention concerns the method, wherein said first homodimeric antibody and/or second homodimeric antibody binds CD3.

According to an embodiment, the invention concerns the method, wherein said first homodimeric antibody and/or second homodimeric antibody binds CD25.

According to an embodiment, the invention concerns the method, wherein said first homodimeric antibody binds CD3 and said second homodimeric antibody binds CD25.

According to an embodiment, the invention concerns the method, wherein said heterodimeric antibody comprises a sequence of SEQ ID NO:51-69.

According to an embodiment, the invention concerns the method, wherein said heterodimeric antibody comprises a sequence with a least 70%, preferably 75%, more preferred 80%, preferably 85%, more preferred 90%, preferably 95%, more preferred 97% sequence identity to a sequence of SEQ ID NO:51-69.

According to an embodiment, the invention concerns a heterodimeric antibody obtainable by the method according to the invention.

According to an embodiment, the invention concerns a heterodimeric antibody comprising:

A first heavy chain comprising a first Fc region, said first Fc region comprising a first CH3 region selected from the group consisting of a human IgG2 CH3 region and a human IgG4 CH3 region, but with an amino acid substitution at position 405 (EU numbering), and

A second heavy chain comprising a second Fc region, said second Fc region comprising a second CH3 region selected from the group consisting of a human IgG2 CH3 region and a human IgG4 CH3, but with an amino acid substitution at position 409 (EU numbering);

wherein the sequences of the first and second CH3 regions are different.

According to an embodiment, the invention concerns a heterodimeric antibody comprising:

A first heavy chain comprising a first Fc region, said first Fc region comprising a first CH3 region selected from the group consisting of a human IgG2 CH3 region and a human IgG4 CH3 region, and

A second heavy chain comprising a second Fc region, said second Fc region comprising a second CH3 region selected from the group consisting of a human IgG2 CH3 region and a human IgG4 CH3, but with an amino acid substitution at position 405 and an amino acid substitution at position 409 (EU numbering);

wherein the sequences of the first and second CH3 regions are different.

According to an embodiment, the invention concerns the heterodimeric antibody, wherein said first and/or second CH3 region is of human IgG2.

According to an embodiment, the invention concerns the heterodimeric antibody, wherein said first and/or second CH3 region is of human IgG4.

According to an embodiment, the invention concerns the heterodimeric antibody, wherein said first Fc region comprises an Arg at position 409 (EU numbering).

According to an embodiment, the invention concerns the heterodimeric antibody, wherein said second Fc region comprises a Leu at position 405 (EU numbering).

According to an embodiment, the invention concerns the heterodimeric antibody, wherein said first Fc region comprises an Arg at position 409 (EU numbering) and said second Fc region comprises a Leu at position 405 (EU numbering).

According to an embodiment, the invention concerns the heterodimeric antibody, further comprising an amino acid substitution at position 234 (EU numbering).

According to an embodiment, the invention concerns the heterodimeric antibody, further comprising an amino acid substitution at position 235 (EU numbering).

According to an embodiment, the invention concerns the heterodimeric antibody, further comprising an amino acid substitution at position 234 and/or position 235 (EU numbering).

According to an embodiment, the invention concerns the heterodimeric antibody, wherein said heterodimeric antibody comprises an Ala at position 234 (EU numbering).

According to an embodiment, the invention concerns the heterodimeric antibody, wherein said heterodimeric antibody comprises an Ala at position 235 (EU numbering).

According to an embodiment, the invention concerns the heterodimeric antibody, further comprising an amino acid substitution at position 322 (EU numbering).

According to an embodiment, the invention concerns the heterodimeric antibody, wherein said heterodimeric antibody comprises an Ala at position 322 (EU numbering).

According to an embodiment, the invention concerns the heterodimeric antibody, further comprising an amino acid substitution at position 228 (EU numbering).

According to an embodiment, the invention concerns the heterodimeric antibody, wherein said heterodimeric antibody comprises a Pro at position 228 (EU numbering).

According to an embodiment, the invention concerns the heterodimeric antibody, further comprising an amino acid substitution at position 297 (EU numbering).

According to an embodiment, the invention concerns the heterodimeric antibody, wherein said heterodimeric antibody comprises an Ala at position 297 (EU numbering).

According to an embodiment, the invention concerns the heterodimeric antibody, wherein said heterodimeric antibody comprises a hinge region.

According to an embodiment, the invention concerns the heterodimeric antibody, wherein said hinge region is selected from naturally occurring and modified hinge regions.

According to an embodiment, the invention concerns the heterodimeric antibody, wherein said hinge region is selected from the group consisting of naturally occurring IgG1, IgG2, IgG3 and IgG4 hinge regions.

According to an embodiment, the invention concerns the heterodimeric antibody, wherein said hinge region is selected from the group consisting of modified IgG1, IgG2, IgG3 and IgG4 hinge regions.

According to an embodiment, the invention concerns the heterodimeric antibody, wherein said hinge region is having at least 80%, alternatively at least 85%, alternatively at least 90%, alternatively at least 95% sequence similarity to a hinge region selected from the group consisting of naturally occurring IgG1, IgG2, IgG3 and IgG4 hinge regions.

According to an embodiment, the invention concerns the heterodimeric antibody, wherein said heterodimeric antibody comprises a hinge region selected from an IgG1 hinge region or an IgG4 hinge region with a S228P mutation.

According to an embodiment, the invention concerns the heterodimeric antibody, wherein said hinge region comprises two disulfide bonds.

According to an embodiment, the invention concerns the heterodimeric antibody, wherein said hinge region comprises a sequence of CPAP.

According to an embodiment, the invention concerns the heterodimeric antibody, wherein said hinge region comprises a sequence of SEQ ID NO: 72.

According to an embodiment, the invention concerns the heterodimeric antibody, wherein said hinge region comprises a sequence of SEQ ID NO: 72 with one amino acid substitution, one amino acid modification, one amino acid deletion or one amino acid addition.

According to an embodiment, the invention concerns the heterodimeric antibody, wherein said heterodimeric antibody comprises a CH1 region and said CH1 region comprises a Cys within the first 20 amino acid residues of said CH1 region.

According to an embodiment, the invention concerns the heterodimeric antibody, wherein said heterodimeric antibody comprises a CH1 region and said CH1 region comprises a Cys at position 14 of said CH1 region.

According to an embodiment, the invention concerns the heterodimeric antibody, wherein said heterodimeric antibody comprises a CH1 region selected from the group consisting of IgG2 CH1 regions, IgG3 CH1 regions and IgG4 CH1 regions.

According to an embodiment, the invention concerns the heterodimeric antibody, wherein said heterodimeric antibody comprises a linker.

According to an embodiment, the invention concerns the heterodimeric antibody, wherein said first heavy chain and/or said second heavy chain is chimeric, humanized, or human.

According to an embodiment, the invention concerns the heterodimeric antibody, wherein said first and second heavy chains are full-length heavy chains.

According to an embodiment, the invention concerns the heterodimeric antibody, wherein said first and second heavy chains bind different epitopes.

According to an embodiment, the invention concerns the heterodimeric antibody, wherein said first and second heavy chains are full-length heavy chains of two antibodies that bind different epitopes.

According to an embodiment, the invention concerns the heterodimeric antibody, further comprising two full-length light chains.

According to an embodiment, the invention concerns a pharmaceutical composition comprising a heterodimeric antibody according to the invention and an excipient, such as a pharmaceutically-acceptable carrier.

According to an embodiment, the invention concerns the method, wherein step c) further comprises co-expressing one or more nucleic-acid constructs encoding a light-chain in said host cell.

According to an embodiment, the invention concerns the heterodimeric antibody, wherein said first and/or second heavy chains bind CD3.

According to an embodiment, the invention concerns the heterodimeric antibody, wherein said first and/or second heavy chains bind CD25.

According to an embodiment, the invention concerns the heterodimeric antibody, wherein said first heavy chain binds CD3 and said second heavy chain binds CD25.

According to an embodiment, the invention concerns the heterodimeric antibody, wherein said heterodimeric antibody comprises a sequence selected from the group consisting of SEQ ID NO:51-69.

According to an embodiment, the invention concerns the heterodimeric antibody, wherein said heterodimeric antibody comprises a sequence with a least 70%, preferably 75%, more preferred 80%, preferably 85%, more preferred 90%, preferably 95%, more preferred 97% sequence identity to a sequence selected from the group consisting of SEQ ID NO:51-69.

According to an embodiment, the invention concerns a bispecific antibody or fragment thereof comprising a first antigen binding site capable of binding to a first antigen and a second antigen binding site capable of binding to a second antigen, wherein said first antigen binding site is comprised in a Fab fragment and said second antigen binding site is comprised in a moiety selected from the group consisting of scFv, antibody fragments and protein moiety, wherein said moiety being attached to the light chain of said Fab fragment.

According to an embodiment, the invention concerns the bispecific antibody or fragment thereof, wherein said moiety is a scFv.

According to an embodiment, the invention concerns the bispecific antibody, wherein said moiety is attached to the C-terminus or N-terminus of the light chain of said Fab fragment.

According to an embodiment, the invention concerns the bispecific antibody, wherein said moiety is attached to the C-terminus of the light chain of said Fab fragment.

According to an embodiment, the invention concerns the bispecific antibody, wherein said Fab fragment is derived from an IgG or an IgM.

According to an embodiment, the invention concerns the bispecific antibody, wherein said Fab fragment is derived from an IgG.

According to an embodiment, the invention concerns the bispecific antibody, wherein said Fab fragment is derived from an IgG selected from the group consisting of IgG1, IgG2, IgG3 and IgG4.

According to an embodiment, the invention concerns the bispecific antibody, wherein said Fab fragment is derived from an IgG2 or an IgG4.

According to an embodiment, the invention concerns the bispecific antibody, wherein said Fab fragment is derived from an IgG2 or an IgG4 as defined according to the invention.

According to an embodiment, the invention concerns the bispecific antibody, wherein said moiety is attached to a light chain of an IgG2 or IgG4 as defined according to the invention.

According to an embodiment, the invention concerns the bispecific antibody, wherein said first antigen is CD3 or CD25.

According to an embodiment, the invention concerns the bispecific antibody, wherein said second antigen is CD3 or CD25.

According to an embodiment, the invention concerns a heterodimeric antibody, preferably according to the invention, wherein said heterodimeric antibody comprises a human IgG4 Fc domain, wherein said heterodimeric antibody binds to two or more different targets and wherein said heterodimeric antibody further comprises a component selected from
(i) a Fab,
(ii) a scFv,
(iii) a nanobody,
(iv) a variable heavy homodimer (VHH), and/or
(v) an antibody fragment.

According to an embodiment, the invention concerns the heterodimeric antibody further comprising a S228P mutation (EU numbering) in the hinge region.

According to an embodiment, the invention concerns the heterodimeric antibody, wherein said heterodimeric antibody comprises
(i) a first Fc domain comprising native sequence in position 405-409 (EU numbering) of the CH3 domain and
(ii) a second Fc domain comprising F405L and R409K mutations (EU numbering) in the CH3 domain.

According to an embodiment, the invention concerns the heterodimeric antibody, wherein said heterodimeric antibody comprises a Fc-null mutation selected from the group consisting of V234A, N297A, and K322A (EU numbering).

According to an embodiment, the invention concerns a heterodimeric antibody, wherein said heterodimeric antibody comprises a human IgG2 Fc domain,
wherein said heterodimeric antibody binds to two or more different targets and
wherein said heterodimeric antibody further comprises a component selected from
   (i) a Fab,
   (ii) a scFv,
   (iii) a nanobody,
   (iv) a variable heavy homodimer (VHH), and/or
   (v) an antibody fragment.

According to an embodiment, the invention concerns the heterodimeric antibody, wherein the native IgG2 hinge is replaced by an IgG1 hinge or an IgG4 hinge with S228P mutation (EU numbering).

According to an embodiment, the invention concerns the heterodimeric antibody, wherein said native IgG2 hinge comprises the sequence CCVECPPCPAP (SEQ ID NO:85), said IgG1 hinge comprises the sequence DKTHTCPPCPAP (SEQ ID NO:86) and said IgG4 hinge with S228P mutation comprises the sequence YGPPCPPCPAP (SEQ ID NO:87).

According to an embodiment, the invention concerns the heterodimeric antibody, wherein said heterodimeric antibody comprises
(i) a first Fc domain comprising a K409R mutation (EU numbering) in the CH3 domain and
(ii) a second Fc domain comprising a F405L mutation (EU numbering) in the CH3 domain.

According to an embodiment, the invention concerns the heterodimeric antibody, wherein said heterodimeric antibody comprises a Fc-null mutation selected from the group consisting of F234A, L235A, N297A, and K322A (EU numbering).

According to an embodiment, the invention concerns an IgG2, IgG3 or IgG4 molecule that comprises a light chain and an antibody domain and/or protein fused to the C-terminus of said light chain, wherein said antibody domain is selected from
(i) a Fab,
(ii) a scFv,
(iii) a nanobody,
(iv) a variable heavy homodimer (VHH), and/or
(v) an antibody fragment.

According to an embodiment, the invention concerns the IgG2, IgG3 or IgG4 molecule further comprising a linker.

According to an embodiment, the invention concerns the IgG2, IgG3 or IgG4 molecule, wherein said linker comprises a GGGGS (SEQ ID NO: 71) repeat.

According to an embodiment, the invention concerns the IgG2, IgG3 or IgG4 molecule, wherein said linker comprises a number of GGGGS (SEQ ID NO: 71) repeats selected from 1, 2, 3, 4, 5, 6, 7 and 8 repeats, preferably 4-6 repeats.

According to an embodiment, the invention concerns the IgG2, IgG3 or IgG4 molecule, wherein the IgG2, IgG3 or IgG4 molecule comprises a CH1 region and said CH1 region comprises a Cys at position 14 of said CH1 region.

Immunoglobulins are glycoproteins composed of one or more units, each containing four polypeptide chains: two identical heavy chains (HCs) and two identical light chains (LCs). The amino terminal ends of the polypeptide chains show considerable variation in amino acid composition and are referred to as the variable (V) regions to distinguish them from the relatively constant (C) regions. Each light chain consists of one variable domain, VL, and one constant domain, CL. The heavy chains consist of a variable domain, VH, and three constant domains CH1, CH2 and CH3. Heavy and light chains are held together by a combination of non-covalent interactions and covalent interchain disulfide bonds, forming a bilaterally symmetric structure. The V regions of H and L chains comprise the antigen-binding sites of the immunoglobulin (Ig) molecules. Each Ig monomer contains two antigen-binding sites and is said to be bivalent.

The Fab contains one complete L chain in its entirety and the V and CH1 portion of one H chain. The Fab can be further divided into a variable fragment (Fv) composed of the VH and VL domains, and a constant fragment (Fb) composed of the CL and CH1 domains.

The H chain constant domain is generally defined as CH1-CH2-CH3 (IgG, IgA, IgD) with an additional domain (CH4) for IgM and IgE. As described above, the CH1 domain is located within the F(ab) region whereas the remaining CH domains (CH2-CH3 or CH2-CH4) comprise the Fc fragment. This Fc fragment defines the isotype and subclass of the immunoglobulin.

CH3 domain: The terms CH3 domain and CH3 region are used interchangeable herein.

CH1 domain: The terms CH1 domain and CH1 region are used interchangeable herein.

Hinge region: The hinge region is the area of the heavy chains between the first and second C region domains and is held together by disulfide bonds. A hinge region typically comprises between 10 and 30 amino acid residues. IgG hinge region sequences might be defined as the underlined sequences below:

| | Hinge |
|---|---|
| Construct | region sequence (underlined) |
| IgG1 | KS--CDKTHT-----------CPPCPAP |
| IgG2 | K----------------CCVECPPCPAP |
| IgG3 | KTPLGDTTHTPEPKSCDTPPPCPRCPAP |
| IgG4 | KY--G--------------PPCPSCPAP |
| V-IGG2, -A, -B | KD---------------KTHTCPPCPAP |
| (contains IgG1 | |
| hinge) | |
| V-IGG2-C, -D, -E | K----------------YGPPCPPCPAP |
| (contains IgG4 | |
| hinge) | |

Linker: A linker might be a peptide linker or a non-peptide linker. An example of a peptide linker is a Gly/Ser peptide linker comprising a five amino acid residue unit, GGGGS (SEQ ID NO:71), that can be repeated a suitable amount of times. A linker might be a naturally occurring linker or a synthetically produced linker. A linker might occur naturally in a molecule or might be synthetically added to a molecule.

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen-binding fragment of an antibody may be produced by any means. For example, an antigen-binding fragment of an antibody may be enzymatically or chemically produced by fragmentation of an intact antibody and/or it may be recombinantly produced from a gene encoding the partial antibody sequence. Alternatively or additionally, antigen-binding fragment of an antibody may be wholly or partially synthetically produced. An antigen-binding fragment of an antibody may optionally comprise a single chain antibody fragment. Alternatively or additionally, an antigen-binding fragment of an antibody may comprise multiple chains that are linked together, for example, by disulfide linkages. An antigen-binding fragment of an antibody may optionally comprise a multi-molecular complex. A functional antibody fragment typically comprises at least about 50 amino acids and more typically comprises at least about 200 amino acids.

Antibody or fragment thereof: As used herein, an “antibody or fragment thereof” refers to an antibody or antibody fragment as defined above.

Humanized antibodies: Humanized antibodies are antibodies from non-human species whose protein sequences have been modified to increase their similarity to antibody variants produced naturally in humans.

Nanobodies: Nanobodies are known in the art and may comprise the variable regions of a heavy chain antibody as found in the Camelidae family. Unlike other antibodies camelid antibodies lack a light chain and are composed of two identical heavy chains. Heavy chain antibodies are also found in some sharks and such antibodies can also provide the basis for nanobodies. Nanobodies are usually smaller than scFv.

Single-chain Fv (scFv): Single-chain Fvs (scFvs) are widely known and used in the art. A single-chain Fv is a fusion protein of the variable regions of the heavy (VH) and light chains (VL) of immunoglobulins, often connected by a short linker peptide (see, e.g., see, e.g., Benny K. C. Lo (ed.), Antibody Engineering—Methods and Protocols, Humana Press 2004, and references cited therein).

The light chains of immunoglobulins can be a lambda (λ) or kappa (κ) chain. Lambda light chain sequence and kappa light chain sequence might be defined as below:

| Construct | Sequence |
|---|---|
| IGKC HUMAN | RTVAAPSVFIFPPSDEQLKSGTASVVCLLNNFY |
| Immunoglobulin | PREAKVQWKVDNALQSGNSQESVTEQDSKDSTY |
| kappa constant | SLSSTLTLSKADYEKHKVYACEVTHQGLSSPVT |
| | KSFNRGEC (SEQ ID NO: 88) |
| IGLC1 HUMAN | GQPKANPTVTLFPPSSEELQANKATLVCLISDF |
| Immunoglobulin | YPGAVTVAWKADGSPVKAGVETTKPSKQSNNKY |
| lambda constant | AASSYLSLTPEQWKSHRSYSCQVTHEGSTVEKT |
| 1 | VAPTECS (SEQ ID NO: 89) |
| IGLC2 HUMAN | GQPKAAPSVTLFPPSSEELQANKATLVCLISDF |
| Immunoglobulin | YPGAVTVAWKADSSPVKAGVETTTPSKQSNNKY |
| lambda constant | AASSYLSLTPEQWKSHRSYSCQVTHEGSTVEKT |
| 2 | VAPTECS (SEQ ID NO: 90) |
| IGLC3 HUMAN | GQPKAAPSVTLFPPSSEELQANKATLVCLISDF |
| Immunoglobulin | YPGAVTVAWKADSSPVKAGVETTTPSKQSNNKY |
| lambda constant | AASSYLSLTPEQWKSHKSYSCQVTHEGSTVEKT |
| 3 | VAPTECS (SEQ ID NO: 91) |
| IGLC6 HUMAN | GQPKAAPSVTLFPPSSEELQANKATLVCLISDF |
| Immunoglobulin | YPGAVKVAWKADGSPVNTGVETTTPSKQSNNKY |
| lambda constant | AASSYLSLTPEQWKSHRSYSCQVTHEGSTVEKT |
| 6 | VAPAECS (SEQ ID NO: 92) |
| IGLC7 HUMAN | GQPKAAPSVTLFPPSSEELQANKATLVCLVSDF |
| Immunoglobulin | NPGAVTVAWKADGSPVKVGVETTKPSKQSNNKY |
| lambda constant | AASSYLSLTPEQWKSHRSYSCRVTHEGSTVEKT |
| 7 | VAPAECS (SEQ ID NO: 93) |

Throughout this application, “EU numbering” refers to numbering according to the EU Index.

A psycho-immune disorder may be understood as a psychiatric disorder caused and/or affected by inflammation, and/or an inflammatory disorder caused and/or affected by a psychiatric disorder.

A mood disorder may be defined as a general emotional state or mood that is distorted or inconsistent with the circumstances and interferes with a subject's ability to function. A subject may be extremely sad, empty or irritable (depressed), or may have periods of depression alternating with being excessively happy (mania). A mood disorder may also be understood as a disorder classified by the American Psychiatric Association's Diagnostic and Statistical Manual of Mental Disorders (DSM-V).

According to an embodiment, the invention concerns an antibody or fragment thereof comprising a variable light chain sequence with at least 82% identity to a human germline variable light chain sequence.

According to an embodiment, the invention concerns an antibody or fragment thereof comprising a variable light chain sequence with at least 83%, alternatively at least 84%, alternatively at least 85%, alternatively at least 86%, alternatively at least 87% identity to a human germline variable light chain sequence.

According to an embodiment, the invention concerns an antibody or fragment thereof comprising a variable heavy chain sequence with at least 80% identity to a human germline variable heavy chain sequence.

According to an embodiment, the invention concerns antibody or fragment thereof comprising a variable heavy chain sequence with at least 80%, alternatively at least 81%, alternatively at least 82%, alternatively at least 83%, alternatively at least 84%, alternatively at least 85%, alternatively at least 86%, alternatively at least 87%, alternatively at least 88%, alternatively at least 89% identity to a human germline variable heavy chain sequence.

According to an embodiment, the invention concerns an antibody or fragment thereof comprising a variable light chain sequence with at least 70% identity to a human germline variable light chain sequence

According to an embodiment, the invention concerns an antibody or fragment thereof comprises a variable light chain sequence with at least 70%, alternatively at least 75%, alternatively at least 80%, alternatively at least 81%, alternatively at least 82%, alternatively at least 83%, alternatively at least 84% identity to a human germline variable light chain sequence.

According to an embodiment, the invention concerns an antibody or fragment thereof comprises a variable heavy chain sequence with at least 70% identity to a human germline variable heavy chain sequence.

According to an embodiment, the invention concerns an antibody or fragment thereof comprises a variable heavy chain sequence with at least 70%, alternatively at least 75%, alternatively at least 80%, alternatively at least 81%, alternatively at least 82%, alternatively at least 83%, alternatively at least 84%, alternatively at least 85%, alternatively at least 86% identity to a human germline variable heavy chain sequence.

According to an embodiment, the invention concerns an antibody or fragment thereof, wherein the antigen contact residues are determined using IMGT.

### FIGURES

FIG. 1a shows a schematic representation of monospecific and bispecific antibodies. VIT-100 is a 4 chain IgG anti-CD3 monospecific antibody, VIT-101 is a 4 chain IgG anti-CD25 monospecific antibody, VIT-102 is a 4 chain IgG antiCD3/anti-CD25 bispecific antibody, VIT-103 is a 2 chain scFv-Fc anti-CD3 monospecific antibody, VIT-104 is a 2 chain scFv-Fc anti-CD25 monospecific antibody and VIT-105 is 2 chain scFv-Fc anti-CD3/anti-CD25 bispecific antibody.

FIG. 1b shows a schematic representation of bispecific antibodies. VIT-106-108 are 4 chain IgG-scFv fusion anti-CD3/anti-CD25 bispecific antibodies and VIT-109-111 are 2 chain scFv-Fc-scFv anti-CD3/anti-CD25 bispecific antibodies.

FIG. 2a and FIG. 2b shows the SPR sensorgrams for VP001 and VP002 to human CD3de immobilized onto the surface of CM5 sensor chip.

FIG. 2a: VP001 (Teplizumab H:C114S) had a KD of 5.2 nM

FIG. 2b: VP002 (huOKT3 H3L3) had a KD of 12.4 nM.

FIG. 3a and FIG. 3b shows the SPR sensorgrams for VP007 and VP008 to human CD25 immobilized onto the surface of CM5 sensor chip.

FIG. 3a: VP007 (chimeric Basiliximab) had a KD of 0.8 nM.

FIG. 3b: VP008 (huBasiliximab H2L2) had a KD of 1.2 nM.

FIGS. 4a and 4b shows hydrophobic interaction chromatography (HIC) profiles documenting that VP021 and VP022 efficiently formed heterodimers.

FIG. 5 shows CD8+ T cells proliferation without the addition of Tregs and in the cocultures with Tregs in response to treatment with different compounds. VP008-PUR and VP021-2 treatment led to a decrease in CD8⁺ T cell proliferation in the presence of Tregs in a dose-dependent manner. VP022 also showed some modest decrease in CD8⁺ T cell proliferation in the presence of Tregs. The same trends were not observed when the test articles were added to CD8+ T cell in the absence of Treg cells.

FIG. 6 shows the VIT-112 format.

FIG. 7 shows additional formats for antibodies according to the invention comprising a nanobody binding to CD3.

FIGS. 8a and 8b show the SPR sensorgrams for VP100 and VP101 respectively to human CD25 immobilized onto the surface of CM5 sensor chip. VP100 (ch7G7) had a KD1 of 3.8 nM, and VP101 (hu7G7) had an apparent KD1 of <1 nM.

FIG. 9a shows the results from an in vitro study to assess Treg activation in a gut mucosal-like environment, where test articles were assessed for their ability to affect neuropilin expression in CD25+FoxP3+ Tregs at 24 hours.

FIG. 9b shows the results from an in vitro study to assess Treg activation in a gut mucosal-like environment, where test articles were assessed for their ability to affect IL-10 expression in CD25+FoxP3+ Tregs at 24 hours.

FIG. 9c shows the results from an in vitro study to assess Treg activation in a gut mucosal-like environment, where test articles were assessed for their ability to affect LAP expression in CD25+FoxP3+ Tregs at 24 hours.

FIG. 10a shows the results from an in vitro study to assess Treg activation in a gut mucosal-like environment, where test articles were assessed for their ability to affect neuropilin expression in CD25+FoxP3+ Tregs at 72 hours.

FIG. 10b shows the results from an in vitro study to assess Treg activation in a gut mucosal-like environment, where test articles were assessed for their ability to affect IL-10 expression in CD25+FoxP3+ Tregs at 72 hours.

FIG. 10c shows the results from an in vitro study to assess Treg activation in a gut mucosal-like environment, where test articles were assessed for their ability to affect LAP expression in CD25+FoxP3+ Tregs at 72 hours.

FIG. 11a shows the results from an in vitro study to assess Treg activation in a gut mucosal-like environment, where test articles were assessed for their ability to sustain CD25+FoxP3+ Tregs as a proportion of CD4+ T cells at 24 hours.

FIG. 11b shows the results from an in vitro study to assess Treg activation in a gut mucosal-like environment, where test articles were assessed for their ability to sustain CD25+FoxP3+ Tregs as a proportion of CD4+ T cells at 72 hours.

FIG. 12 shows the schematic of an animal study to assess how CD3/CD25 antibodies can affect depression and Experimental Autoimmune Encephalomyelitis (EAE) using huCD3e×huCD25 transgenic mice.

FIGS. 13a and 13b show the SPR sensorgrams for VP103 (chimeric alpaca anti-CD3 VHH F10-human IgG4) to human CD3de and cynomolgus CD3de immobilized onto the surface of CM5 sensor chip. The KD of VP103 was 3.7 nM for human CD3de and 2.8 nM for cynomolgus CD3de.

FIGS. 14a and 14b show the SPR sensorgrams for VP104 (Humanized F10A VHH (C55A)-IgG4) for recombinant human CD3de (delta epsilon subunits) and recombinant cynomolgus CD3de (delta epsilon subunits) were assessed by SPR on a Biacore T200 (GE Healthcare). The KD of VP104 was 14.2 nM for human CD3de and 13.3 nM for cynomolgus CD3de.

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Example 1: Design of Humanized Anti-CD3 Sequences

A proposed sequence for a humanized anti-CD3 murine mAb muromomab (OKT3) is hereby provided as follows.

The sequence of anti-CD3 murine mAb muromomab (OKT3) is shown below, antigen contact residues are shown in bold and IMGT CDR sequences are underlined.

VL:
(SEQ ID NO: 1)
QIVLICISPAIMSASPGEKVTMTCSASSSVSYMNWYQQKSGTSPKRWIYD
TSKLASGVPAHFRGSGSGTSYSLTISGMEAEDAATYYCQQWSSNPFTFGS
GTKLEIN
VH:
(SEQ ID NO: 6)
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSS

All contact residues were retained in the humanized sequences. It was noted that the previous humanized OKT3 sequence found in Teplizumab did not retain the non-CDR contact residue H:K82 (IMGT numbering).

All IMGT CDR residues were retaining with the exception of an unpaired H:Cys114 (IMGT numbering) in H:CDR3, which was mutated to Ser in the humanized constructs. The Cys was not a contact residue and it is speculated that it causes aggregation of the humanized products.

Humanizing mutations were made based on identity to human germline sequence or prevalence in human antibody sequences. Final constructs are presented in Table 1 and Table 2.

TABLE 1
Anti-CD3 VL sequences

| | | | Predicted | |
|---|---|---|---|---|
| | | % human | immunogenic | |
| Construct | Sequence | germline | peptides | Notes |
| mOKT3 VL | QIVLTQSPAIMSAS | 61.1% | QIVLTQSPAIMSASP | |
| | PGEKVTMTCSASSS | IGKV3- | | |
| | VSYMNWYQQKSG | 11*01 | | |
| | TSPKRWIYDTSKLA | | | |
| | SGVPAHFRGSGSG | | | |
| | TSYSLTISGMEAED | | | |
| | AATYYCQQWSSNP | | | |
| | FTFGSGTKLEIN | | | |
| | (SEQ ID NO: 1) | | | |
| Teplizuma | DIQMTQSPSSLSAS | 81.1% | none | |
| b VL | VGDRVTITCSASSS | IGKV1- | | |
| | VSYMNWYQQTPG | 33*01 | | |
| | KAPKRWIYDTSKLA | | | |
| | SGVPSRFSGSGSGT | | | |
| | DYTFTISSLQPEDIA | | | |
| | TYYCQQWSSNPFT | | | |
| | FGQGTKLQIT (SEQ | | | |
| | ID NO: 2) | | | |
| VCD3-L1 | DIQMTQSPSSLSAS | 83.2% | none | Based on |
| | VGDRVTITCQASSS | IGKV1- | | germline |
| | VSYMNWYQQKPG | 33*01 | | IGKV1-33 |
| | KAPKRWIYDTSKLA | | | |
| | SGVPSRFSGSGSGT | | | |
| | DYTFTISSLQPEDIA | | | |
| | TYYCQQWSSNPFT | | | |
| | FGQGTKLEIK (SEQ | | | |
| | ID NO: 3) | | | |
| VCD3-L2 | EIVLTQSPATLSLSP | 87.4% | EIVLTQSPATLSLSP | Based on |
| | GERATLSCRASSSV | IGKV3- | QAPRRLIYDTSKRAT | germline |
| | SYMNWYQQKPGQ | 11*01 | | IGKV3-11 |
| | APRRLIYDTSKRAT | | | |
| | G I PARFSGSGSGTD | | | |
| | YTLTISSLEPEDAAV | | | |
| | YYCQQWSSNPFTF | | | |
| | GQGTKLEIK (SEQ | | | |
| | ID NO: 4) | | | |
| VCD3-L3 | EIQLTQSPATLSLSP | 84.2% | none | Removed |
| | GERATLSCRASSSV | IGKV3- | | immunogenic |
| | SYMNWYQQKPGQ | 11*01 | | epitopes |
| | APRRWIYDTSKLAT | | | In V2 |
| | GIPARFSGSGSGTD | | | |
| | YTLTISSLEPEDAAV | | | |
| | YYCQQWSSNPFTF | | | |
| | GQGTKLEIK (SEQ | | | |
| | ID NO: 5) | | | |

TABLE 2
Anti-CD3 VH sequences

| | | | Predicted | |
|---|---|---|---|---|
| | | % human | immunogenic | |
| Construct | Sequence | germline | peptides | Notes |
| mOKT3 VH | QVQLQQSGAELA | 72.4% | GYITTRYTMHINVK | |
| | RPGASVKMSCKA | IGHV1- | QR | |
| | SGYTFTRYTMHW | 46*01 | | |
| | VKQRPGQGLEWI | | | |
| | GYINPSRGYTNYN | | | |
| | QKFKDKATLTTDK | | | |
| | SSSTAYMQLSSLTS | | | |
| | EDSAVYYCARYYD | | | |
| | DHYCLDYWGQGT | | | |
| | TLTVSS (SEQ ID | | | |
| | NO: 6) | | | |
| Teplizumab | QVQLVQSGGGVV | 72.4% | RYTMHWVRQAPGK | |
| VH | QPGRSLRLSCKAS | IGHV3- | GL | |
| | GYTFTRYTMHWV | 30*10 | KNTAFLOMDSLRPE | |
| | RQAPGKGLEWIG | | D | |
| | YINPSRGYTNYNQ | | | |
| | KVKDRFTISRDNS | | | |
| | KNTAFLQMDSLRP | | | |
| | EDTGVYFCARYYD | | | |
| | DHYCLDYWGQGT | | | |
| | PVTVSS (SEQ ID | | | |
| | NO: 7) | | | |
| VCD3-H1 | QVQLVQSGGGVV | 81.6% | RYTMHWVRQAPGK | Based on |
| | QPGRSLRLSCAAS | IGHV3- | GL | germline |
| | GYTFTRYTMHWV | 30*10 | KNTAYLQMNSLRAE | IGHV3-30 |
| | RQAPGKGLEWVG | | D | |
| | YINPSRGYTNYTD | | | |
| | SVKGRFTISTDKSK | | | |
| | NTAYLQMNSLRA | | | |
| | EDTAVYYCARYYD | | | |
| | DHYSLDYWGQGT | | | |
| | TVTVSS (SEQ ID | | | |
| | NO: 8) | | | |
| VCD3-H2 | QVQLVQSGAEVK | 89.8% | RYTMHVRQAPG | Based on |
| | KPGASVKVSCKAS | IGHV1- | QGL | germline |
| | GYTFTRYTMHWV | 46*01 | | IGHV1-46 |
| | RQAPGQGLEWM | | | |
| | GYINPSRGYTNYN | | | |
| | QKFQGRVTMTTD | | | |
| | KSTSTAYMELSSLR | | | |
| | SEDTAVYYCARYY | | | |
| | DDHYSLDYWGQG | | | |
| | TTVTVSS (SEQ ID | | | |
| | NO: 9) | | | |
| VCD3-H3 | QVQLVQSGAEVK | 88.8% | none | Removed |
| | KPGASVKVSCKAS | IGHV1- | | immunogenic |
| | GYTFTRYTMHWV | 46*01 | | epitopes |
| | RQSPGQGLEWM | | | in V2 |
| | GYINPSRGYTNYN | | | |
| | QKFQGRVTMTTD | | | |
| | KSTSTAYMELSSLR | | | |
| | SEDTAVYYCARYY | | | |
| | DDHYSLDYWGQG | | | |
| | TTVTVSS (SEQ ID | | | |
| | NO: 10) | | | |

Single chain variable fragments (scFv) were designed based on a VH-VL orientation and are presented in Table 3. Additional disulfide stabilization between the VH and VL domains was engineered by substituting Cys at positions VH44-VL100 (Kabat numbering).

TABLE 3
Anti-CD3 scFv sequences

| Construct | Sequence |
|---|---|
| VCD3- | QVQLVQSGGGVVQPGRSLRLSCAASGYTFTRYTMHWVRQAPGKGLEWVGYINPSRGYTNYNDSV |
| H1L1 | KGRFTISTDKSKNTAYLQMNSLRAEDTAVYYCARYYDDHYSLDYWGQGTTVTVSSGGGGSGGGGS |
| | GGGGSGGGGSGGGGSGGGGS |
| | DIQMTQSPSSLSASVGDRVTITCQASSSVSYMNWYQQKPGKAPKRWIYDTSKLASGVPSRFSGSGS |
| | GTDYTFTISSLQPEDIATYYCQQWSSNPFTFGQGTKLEIK (SEQ ID NO: 11) |
| VCD3- | QVQLVQSGGGVVQPGRSLRLSCAASGYTFTRYTMHWVRQAPGKCLEWVGYINPSRGYTNYNDSV |
| H1L1-DS | KGRFTISTDKSKNTAYLQMNSLRAEDTAVYYCARYYDDHYSLDYWGQGTTVTVSSGGGGSGGGGS |
| | GGGGSGGGGSGGGGSGGGGS |
| | DIQMTQSPSSLSASVGDRVTITCQASSSVSYMNWYQQKPGKAPKRWIYDTSKLASGVPSRFSGSGS |
| | GTDYTFTISSLQPEDIATYYCQQWSSNPFTFGCGTKLEIK (SEQ ID NO: 12) |
| VCD3- | QVQLVQSGGGVVQPGRSLRLSCAASGYTFTRYTMHWVRQAPGKGLEWVGYINPSRGYTNYNDSV |
| H1L2 | KGRFTISTDKSKNTAYLQMNSLRAEDTAVYYCARYYDDHYSLDYWGQGTTVTVSSGGGGSGGGGS |
| | GGGGSGGGGSGGGGSGGGGS |
| | EIVLTQSPATLSLSPGERATLSCRASSSVSYMNWYQQKPGQAPRRLIYDTSKRATGIPARFSGSGSGT |
| | DYTLTISSLEPEDAAVYYCQQWSSNPFTFGQGTKLEIK (SEQ ID NO: 13) |
| VCD3- | QVQLVQSGGGVVQPGRSLRLSCAASGYTFTRYTMHWVRQAPGKCLEWVGYINPSRGYTNYNDSV |
| H1L2-DS | KGRFTISTDKSKNTAYLQMNSLRAEDTAVYYCARYYDDHYSLDYWGQGTTVTVSSGGGGSGGGGS |
| | GGGGSGGGGSGGGGSGGGGS |
| | EIVLTQSPATLSLSPGERATLSCRASSSVSYMNWYQQKPGQAPRRLIYDTSKRATGIPARFSGSGSGT |
| | DYTLTISSLEPEDAAVYYCQQWSSNPFTFGCGTKLEIK (SEQ ID NO: 14) |
| VCD3- | QVQLVQSGGGVVQPGRSLRLSCAASGYTFTRYTMHWVRQAPGKGLEWVGYINPSRGYTNYNDSV |
| H1L3 | KGRFTISTDKSKNTAYLQMNSLRAEDTAVYYCARYYDDHYSLDYWGQGTTVTVSSGGGGSGGGGS |
| | GGGGSGGGGSGGGGSGGGGS |
| | EIQLTQSPATLSLSPGERATLSCRASSSVSYMNWYQQKPGQAPRRWIYDTSKLATGIPARFSGSGSGT |
| | DYTLTISSLEPEDAAVYYCQQWSSNPFTFGQGTKLEIK (SEQ ID NO: 15) |
| VCD3- | QVQLVQSGGGVVQPGRSLRLSCAASGYTFTRYTMHWVRQAPGKCLEWVGYINPSRGYTNYNDSV |
| H1L3-DS | KGRFTISTDKSKNTAYLQMNSLRAEDTAVYYCARYYDDHYSLDYWGQGTTVTVSSGGGGSGGGGS |
| | GGGGSGGGGSGGGGSGGGGS |
| | EIQLTQSPATLSLSPGERATLSCRASSSVSYMNWYQQKPGQAPRRWIYDTSKLATGIPARFSGSGSGT |
| | DYTLTISSLEPEDAAVYYCQQWSSNPFTFGCGTKLEIK (SEQ ID NO: 16) |
| VCD3- | QVQLVQSGAEVKKPGASVKVSCKASGYTFTRYTMHWVRQAPGQGLEWMGYINPSRGYTNYNQKF |
| H2L1 | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | DIQMTQSPSSLSASVGDRVTITCQASSSVSYMNWYQQKPGKAPKRWIYDTSKLASGVPSRFSGSGS |
| | GTDYTFTISSLQPEDIATYYCQQWSSNPFTFGQGTKLEIK (SEQ ID NO: 17) |
| VCD3- | QVQLVQSGAEVKKPGASVKVSCKASGYTFTRYTMHWVRQAPGQCLEWMGYINPSRGYTNYNQKF |
| H2L1-DS | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | DIQMTQSPSSLSASVGDRVTITCQASSSVSYMNWYQQKPGKAPKRWIYDTSKLASGVPSRFSGSGS |
| | GTDYTFTISSLQPEDIATYYCQQWSSNPFTFGCGTKLEIK (SEQ ID NO: 18) |
| VCD3- | QVQLVQSGAEVKKPGASVKVSCKASGYTFTRYTMHWVRQAPGQGLEWMGYINPSRGYTNYNQKF |
| H2L2 | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIVLTQSPATLSLSPGERATLSCRASSSVSYMNWYQQKPGQAPRRLIYDTSKRATGIPARFSGSGSGT |
| | DYTLTISSLEPEDAAVYYCQQWSSNPFTFGQGTKLEIK (SEQ ID NO: 19) |
| VCD3- | QVQLVQSGAEVKKPGASVKVSCKASGYTFTRYTMHWVRQAPGQCLEWMGYINPSRGYTNYNQKF |
| H2L2-DS | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIVLTQSPATLSLSPGERATLSCRASSSVSYMNWYQQKPGQAPRRLIYDTSKRATGIPARFSGSGSGT |
| | DYTLTISSLEPEDAAVYYCQQWSSNPFTFGCGTKLEIK (SEQ ID NO: 20) |
| VCD3- | QVQLVQSGAEVKKPGASVKVSCKASGYTFTRYTMHWVRQAPGQGLEWMGYINPSRGYTNYNQKF |
| H2L3 | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIQLTQSPATLSLSPGERATLSCRASSSVSYMNWYQQKPGQAPRRWIYDTSKLATGIPARFSGSGSGT |
| | DYTLTISSLEPEDAAVYYCQQWSSNPFTFGQGTKLEIK (SEQ ID NO: 21) |
| VCD3- | QVQLVQSGAEVKKPGASVKVSCKASGYTFTRYTMHWVRQAPGQCLEWMGYINPSRGYTNYNQKF |
| H2L3-DS | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIQLTQSPATLSLSPGERATLSCRASSSVSYMNWYQQKPGQAPRRWIYDTSKLATGIPARFSGSGSGT |
| | DYTLTISSLEPEDAAVYYCQQWSSNPFTFGCGTKLEIK (SEQ ID NO: 22) |
| VCD3- | QVQLVQSGAEVKKPGASVKVSCKASGYTFTRYTMHWVRQSPGQGLEWMGYINPSRGYTNYNQKF |
| H3L1 | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | DIQMTQSPSSLSASVGDRVTITCQASSSVSYMNWYQQKPGKAPKRWIYDTSKLASGVPSRFSGSGS |
| | GTDYTFTISSLQPEDIATYYCQQWSSNPFTFGQGTKLEIK (SEQ ID NO: 23) |
| VCD3- | QVQLVQSGAEVKKPGASVKVSCKASGYTFTRYTMHWVRQSPGQCLEWMGYINPSRGYTNYNQKF |
| H3L1-DS | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | DIQMTQSPSSLSASVGDRVTITCQASSSVSYMNWYQQKPGKAPKRWIYDTSKLASGVPSRFSGSGS |
| | GTDYTFTISSLQPEDIATYYCQQWSSNPFTFGCGTKLEIK (SEQ ID NO: 24) |
| VCD3- | QVQLVQSGAEVKKPGASVKVSCKASGYTFTRYTMHWVRQSPGQGLEWMGYINPSRGYTNYNQKF |
| H3L2 | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIVLTQSPATLSLSPGERATLSCRASSSVSYMNWYQQKPGQAPRRLIYDTSKRATGIPARFSGSGSGT |
| | DYTLTISSLEPEDAAVYYCQQWSSNPFTFGQGTKLEIK (SEQ ID NO: 25) |
| VCD3- | QVQLVQSGAEVKKPGASVKVSCKASGYTFTRYTMHWVRQSPGQCLEWMGYINPSRGYTNYNQKF |
| H3L2-DS | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIVLTQSPATLSLSPGERATLSCRASSSVSYMNWYQQKPGQAPRRLIYDTSKRATGIPARFSGSGSGT |
| | DYTLTISSLEPEDAAVYYCQQWSSNPFTFGCGTKLEIK (SEQ ID NO: 26) |
| VCD3- | QVQLVQSGAEVKKPGASVKVSCKASGYTFTRYTMHWVRQSPGQGLEWMGYINPSRGYTNYNQKF |
| H3L3 | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIQLTQSPATLSLSPGERATLSCRASSSVSYMNWYQQKPGQAPRRWIYDTSKLATGIPARFSGSGSGT |
| | DYTLTISSLEPEDAAVYYCQQWSSNPFTFGQGTKLEIK (SEQ ID NO: 27) |
| VCD3- | QVQLVQSGAEVKKPGASVKVSCKASGYTFTRYTMHWVRQSPGQCLEWMGYINPSRGYTNYNQKF |
| H3L3-DS | QGRVTMTTDKSTSTAYMELSSLRSEDTAVYYCARYYDDHYSLDYWGQGTTVTVSSGGGGSGGGGS |
| | GGGGSGGGGSGGGGSGGGGS |
| | EIQLTQSPATLSLSPGERATLSCRASSSVSYMNWYQQKPGQAPRRWIYDTSKLATGIPARFSGSGSGT |
| | DYTLTISSLEPEDAAVYYCQQWSSNPFTFGCGTKLEIK (SEQ ID NO: 28) |

### Example 2: Design of Humanized Anti-CD25 Sequences

A proposed sequence for a humanized anti-CD25 chimeric mAb basiliximab is hereby provided as follows.

The sequence of anti-CD25 chimeric mAb basiliximab is shown below, antigen contact residues are shown in bold and IMGT CDR sequences are underlined.

VH:
(SEQ ID NO: 29)
QVQLQQSGTVLARPGASVKMSCKASGYSFTRYWHWIKQRPGQGLEWIGAI
YPGNSDTSYNQKFEGKAKLTAVISASTAYMELSSLTHEDSAVYYCSRDYG
YYFDFWGQGTTLIVSS
VL:
(SEQ ID NO: 32)
QIVSTQSPAIMSASPGEKVTMTCSASSSRSYMQWYQQKPGTSPKRWIYDT
SKLASGVPARFSGSGSGTSYSLTISSMEAEDAATYYCHQRSSYTFGGGTK
LEIK

All contact residues and IMGT CDR sequences were retained in the humanized sequences.

Humanizing mutations were made based on identity to human germline sequence or prevalence in human antibody sequences. Final constructs are presented in Table 4 and Table 5.

TABLE 4
Anti-CD25 VH sequences

| | | | Predicted | |
|---|---|---|---|---|
| | | % human V- | immunogenic | |
| Construct | Sequence | region | peptides | Notes |
| mBasil VH | QVQLQQSGTVLARPGAS | 66.3% | GYSFTRYWMHWKQR | |
| | VKMSCKASGYSFTRYW | HV1-3*01 | | |
| | MHWIKQRPGQGLEWIG | | | |
| | AIYPGNSDTSYNQKFEGK | | | |
| | AKLTAVTSASTAYMELSS | | | |
| | LTHEDSAVYYCSRDYGYY | | | |
| | FDFWGQGTTLTVSS | | | |
| | (SEQ ID NO: 29) | | | |
| VCD25-H1 | QVQLVQSGAGVAKPGA | 83.7% | none | |
| | SVKVSCKASGYSFTRYW | HV1-3*01 | | |
| | MHWVRQPPGQGLEW | | | |
| | MGAIYPGNSDTSYSQKF | | | |
| | EGRVTITADTSASTAYME | | | |
| | LSSLRSEDTAVYYCSRDY | | | |
| | GYYFDFWGQGTTVTVSS | | | |
| | (SEQ ID NO: 30) | | | |
| VCD25-H2 | QVQLVQSGAEVKKPGAS | 86.7% | none | |
| | VKVSCKASGYSFTRYWM | HV1-3*01 | | |
| | HWVRQPPGQRLEWMG | | | |
| | AIYPGNSDTSYSQKFEGR | | | |
| | VTITADTSASTAYMELSSL | | | |
| | RSEDTAVYYCSRDYGYYF | | | |
| | DFWGQGTTVTVSS (SEQ | | | |
| | ID NO: 31) | | | |

TABLE 5
Anti-CD25 VL sequences

| | | | Predicted | |
|---|---|---|---|---|
| | | % human | immunogenic | |
| Construct | Sequence | germline | peptides | Notes |
| mBasil VL | QIVSTQSPAIMSASPGE | 64.5% | none | |
| | KVTMTCSASSSRSYMQ | IGKV6- | | |
| | WYQQKPGTSPKRWIYD | 21*02 | | |
| | TSKLASGVPARFSGSGS | | | |
| | GTSYSLTISSMEAEDAA | | | |
| | TYYCHQRSSYTFGGGTK | | | |
| | LEIK (SEQ ID NO:32) | | | |
| VCD25-L1 | QIVSTQSPDTQSVTPKE | 80.6% | none | |
| | KVTITCRASSSRSYMQ | IGKV6- | | |
| | WYQQKPDQSPKRWIY | 21*02 | | |
| | DTSKSASGVPSRFSGSG | | | |
| | SGTDYTLTINSLEAEDAA | | | |
| | TYYCHQRSSYTFGQGTK | | | |
| | LEIK (SEQ ID NO: 33) | | | |
| VCD25-L2 | EIVLTQSPDFQSVTPKEK | 84.9% | none | |
| | VTITCRASSSRSYMQWY | IGKV6- | | |
| | QQKPDQSPKRLIYDTSK | 21*02 | | |
| | SASGVPSRFSGSGSGTD | | | |
| | YTLTINSLEAEDAATYYC | | | |
| | HQRSSYTFGQGTKLEIK | | | |
| | (SEQ ID NO: 34) | | | |

Single chain variable fragments (scFv) were designed based on a VH-VL and VL-VH orientations and are presented in Table 6. Additional disulfide stabilization between the VH and VL domains was engineered by substituting Cys at positions VH44-VL100 (Kabat numbering).

TABLE 6
Anti-CD25 scFv sequences

| Construct | Sequence |
|---|---|
| VCD25- | QVQLVQSGAGVAKPGASVKVSCKASGYSFTRYWMHWVRQPPGQGLEWMGAIYPGNSDTSYSQK |
| H1L1 | FEGRVTITADTSASTAYMELSSLRSEDTAVYYCSRDYGYYFDFWGQGTTVTVSS |
| | GGGGSGGGGSGGGGSGGGGSGGGGSGGGGS |
| | QIVSTQSPDTQSVTPKEKVTITCRASSSRSYMQWYQQKPDQSPKRWIYDTSKSASGVPSRFSGSGSG |
| | TDYTLTINSLEAEDAATYYCHQRSSYTFGQGTKLEIK (SEQ ID NO: 35) |
| VCD25- | QVQLVQSGAGVAKPGASVKVSCKASGYSFTRYWMHWVRQPPGQGLEWMGAIYPGNSDTSYSQK |
| H1L1-DS | FEGRVTITADTSASTAYMELSSLRSEDTAVYYCSRDYGYYFDFWGQCTIVIVSS |
| | GGGGSGGGGSGGGGSGGGGSGGGGSGGGGS |
| | QIVSTQSPDTQSVTPKEKVTITCRASSSRSYMQWYQQKPDQSPKRWIYDTSKSASGVPSRFSGSGSG |
| | TDYTLTINSLEAEDAATYYCHQRSSYTFGCGTKLEIK (SEQ ID NO: 36) |
| VCD25- | QVQLVQSGAGVAKPGASVKVSCKASGYSFTRYWMHWVRQPPGQGLEWMGAIYPGNSDTSYSQK |
| H1L2 | FEGRVTITADTSASTAYMELSSLRSEDTAVYYCSRDYGYYFDFWGQGTTVTVSS |
| | GGGGSGGGGSGGGGSGGGGSGGGGSGGGGS |
| | EIVLTQSPDFQSVTPKEKVTITCRASSSRSYMQWYQQKPDQSPKRLIYDTSKSASGVPSRFSGSGSGT |
| | DYTLTINSLEAEDAATYYCHQRSSYTFGQGTKLEIK (SEQ ID NO: 37) |
| VCD25- | QVQLVQSGAGVAKPGASVKVSCKASGYSFTRYWMHWVRQPPGQCLEWMGAIYPGNSDTSYSQK |
| H1L2-DS | FEGRVTITADTSASTAYMELSSLRSEDTAVYYCSRDYGYYFDFWGQGTTVTVSS |
| | GGGGSGGGGSGGGGSGGGGSGGGGSGGGGS |
| | EIVLTQSPDFQSVTPKEKVTITCRASSSRSYMQWYQQKPDQSPKRLIYDTSKSASGVPSRFSGSGSGT |
| | DYTLTINSLEAEDAATYYCHQRSSYTFGCGTKLEIK (SEQ ID NO: 38) |
| VCD25- | QVQLVQSGAEVKKPGASVKVSCKASGYSFTRYWMHWVRQPPGQRLEWMGAIYPGNSDTSYSQKF |
| H2L1 | EGRVTITADTSASTAYMELSSLRSEDTAVYYCSRDYGYYFDFWGQGTTVTVSS |
| | GGGGSGGGGSGGGGSGGGGSGGGGSGGGGS |
| | QIVSTQSPDTQSVTPKEKVTITCRASSSRSYMQWYQQKPDQSPKRWIYDTSKSASGVPSRFSGSGSG |
| | TDYTLTINSLEAEDAATYYCHQRSSYTFGQGTKLEIK (SEQ ID NO: 39) |
| VCD25- | QVQLVQSGAEVKKPGASVKVSCKASGYSFTRYWMHWVRQPPGQCLEWMGAIYPGNSDTSYSQKF |
| H2L1-DS | EGRVTITADTSASTAYMELSSLRSEDTAVYYCSRDYGYYFDFWGQGTTVTVSS |
| | GGGGSGGGGSGGGGSGGGGSGGGGSGGGGS |
| | QIVSTQSPDTQSVTPKEKVTITCRASSSRSYMQWYQQKPDQSPKRWIYDTSKSASGVPSRFSGSGSG |
| | TDYTLTINSLEAEDAATYYCHQRSSYTFGCGTKLEIK (SEQ ID NO: 40) |
| VCD25- | QVQLVQSGAEVKKPGASVKVSCKASGYSFTRYWMHWVRQPPGQRLEWMGAIYPGNSDTSYSQKF |
| H2L2 | EGRVTITADTSASTAYMELSSLRSEDTAVYYCSRDYGYYFDFWGQGTTVTVSS |
| | GGGGSGGGGSGGGGSGGGGSGGGGSGGGGS |
| | EIVLTQSPDFQSVTPKEKVTITCRASSSRSYMQWYQQKPDQSPKRLIYDTSKSASGVPSRFSGSGSGT |
| | DYTLTINSLEAEDAATYYCHQRSSYTFGQGTKLEIK (SEQ ID NO: 41) |
| VCD25- | QVQLVQSGAEVKKPGASVKVSCKASGYSFTRYWMHWVRQPPGQCLEWMGAIYPGNSDTSYSQKF |
| H2L2-DS | EGRVTITADTSASTAYMELSSLRSEDTAVYYCSRDYGYYFDFWGQGTTVTVSS |
| | GGGGSGGGGSGGGGSGGGGSGGGGSGGGGS |
| | EIVLTQSPDFQSVTPKEKVTITCRASSSRSYMQWYQQKPDQSPKRLIYDTSKSASGVPSRFSGSGSGT |
| | DYTLTINSLEAEDAATYYCHQRSSYTFGCGTKLEIK (SEQ ID NO: 42) |
| VCD25- | QIVSTQSPDTQSVTPKEKVTITCRASSSRSYMQWYQQKPDQSPKRWIYDTSKSASGVPSRFSGSGSG |
| L1H1 | TDYTLTINSLEAEDAATYYCHQRSSYTFGQGTKLEIK |
| | GGGGSGGGGSGGGGSGGGGSGGGGSGGGGS |
| | QVQLVQSGAGVAKPGASVKVSCKASGYSFTRYWMHWVRQPPGQGLEWMGAIYPGNSDTSYSQK |
| | FEGRVTITADTSASTAYMELSSLRSEDTAVYYCSRDYGYYFDFWGQGTTVTVSS (SEQ ID NO: 43) |
| VCD25- | QIVSTQSPDTQSVTPKEKVTITCRASSSRSYMQWYQQKPDQSPKRWIYDTSKSASGVPSRFSGSGSG |
| L1H1-DS | TDYTLTINSLEAEDAATYYCHQRSSYTFGCGTKLEIK |
| | GGGGSGGGGSGGGGSGGGGSGGGGSGGGGS |
| | QVQLVQSGAGVAKPGASVKVSCKASGYSFTRYWMHWVRQPPGQCLEWMGAIYPGNSDTSYSQK |
| | FEGRVTITADTSASTAYMELSSLRSEDTAVYYCSRDYGYYFDFWGQGTTVTVSS (SEQ ID NO: 44) |
| VCD25- | QIVSTQSPDTQSVTPKEKVTITCRASSSRSYMQWYQQKPDQSPKRWIYDTSKSASGVPSRFSGSGSG |
| L1H2 | TDYTLTINSLEAEDAATYYCHQRSSYTFGQGTKLEIK |
| | GGGGSGGGGSGGGGSGGGGSGGGGSGGGGS |
| | QVQLVQSGAEVKKPGASVKVSCKASGYSFTRYWMHWVRQPPGQRLEWMGAIYPGNSDTSYSQKF |
| | EGRVTITADTSASTAYMELSSLRSEDTAVYYCSRDYGYYFDFWGQGTTVTVSS (SEQ ID NO: 45) |
| VCD25- | QIVSTQSPDTQSVTPKEKVTITCRASSSRSYMQWYQQKPDQSPKRWIYDTSKSASGVPSRFSGSGSG |
| L1H2-DS | TDYTLTINSLEAEDAATYYCHQRSSYTFGCGTKLEIK |
| | GGGGSGGGGSGGGGSGGGGSGGGGSGGGGS |
| | QVQLVQSGAEVKKPGASVKVSCKASGYSFTRYWMHWVRQPPGQRLEWMGAIYPGNSDTSYSQKF |
| | EGRVTITADTSASTAYMELSSLRSEDTAVYYCSRDYGYYFDFWGQCTTVTVSS (SEQ ID NO: 46) |
| VCD25- | EIVLTQSPDFQSVTPKEKVTITCRASSSRSYMQWYQQKPDQSPKRLIYDTSKSASGVPSRFSGSGSGT |
| L2H1 | DYTLTINSLEAEDAATYYCHQRSSYTFGQGTKLEIK |
| | GGGGSGGGGSGGGGSGGGGSGGGGSGGGGS |
| | QVQLVQSGAGVAKPGASVKVSCKASGYSFTRYWMHWVRQPPGQGLEWMGAIYPGNSDTSYSQK |
| | FEGRVTITADTSASTAYMELSSLRSEDTAVYYCSRDYGYYFDFWGQGTTVTVSS (SEQ ID NO: 47) |
| VCD25- | EIVLTQSPDFQSVTPKEKVTITCRASSSRSYMQWYQQKPDQSPKRLIYDTSKSASGVPSRFSGSGSGT |
| L2H1-DS | DYTLTINSLEAEDAATYYCHQRSSYTFGCGTKLEIK |
| | GGGGSGGGGSGGGGSGGGGSGGGGSGGGGS |
| | QVQLVQSGAGVAKPGASVKVSCKASGYSFTRYWMHWVRQPPGQGLEWMGAIYPGNSDTSYSQK |
| | FEGRVTITADTSASTAYMELSSLRSEDTAVYYCSRDYGYYFDFWGQCTTVTVSS (SEQ ID NO: 48) |
| VCD25- | EIVLTQSPDFQSVTPKEKVTITCRASSSRSYMQWYQQKPDQSPKRLIYDTSKSASGVPSRFSGSGSGT |
| L2H2 | DYTLTINSLEAEDAATYYCHQRSSYTFGQGTKLEIK |
| | GGGGSGGGGSGGGGSGGGGSGGGGSGGGGS |
| | QVQLVQSGAEVKKPGASVKVSCKASGYSFTRYWMHWVRQPPGQRLEWMGAIYPGNSDTSYSQKF |
| | EGRVTITADTSASTAYMELSSLRSEDTAVYYCSRDYGYYFDFWGQGTTVTVSS (SEQ ID NO: 49) |
| VCD25- | EIVLTQSPDFQSVTPKEKVTITCRASSSRSYMQWYQQKPDQSPKRLIYDTSKSASGVPSRFSGSGSGT |
| L2H2-DS | DYTLTINSLEAEDAATYYCHQRSSYTFGCGTKLEIK |
| | GGGGSGGGGSGGGGSGGGGSGGGGSGGGGS |
| | QVQLVQSGAEVKKPGASVKVSCKASGYSFTRYWMHWVRQPPGQRLEWMGAIYPGNSDTSYSQKF |
| | EGRVTITADTSASTAYMELSSLRSEDTAVYYCSRDYGYYFDFWGQCTTVTVSS (SEQ ID NO: 50) |

### Example 3: Design of Antibody Heterodimer Platforms

Human IgG2 and IgG4 antibodies have the lowest ADCC and CDC among human IgG antibodies, and were chosen for optimization for creating novel heterodimeric constructs, which are outlined in Table 7.

Heterodimerization of the IgG2 and IgG4 constructs are based on including K409R (on one half-antibody) and F405L (on second antibody) mutations in the CH3 domains (numbering according to the EU Index, referred to as “EU numbering”. Reference https://www.nature.com/articles/nprot.2014.169). Each half antibody is first generated as a single homodimer, then mixed together and allowed to recombine as heterodimers under reducing and oxidizing conditions.

To further reduce any potential ADCC/ADCP/CDC effector functions, LALA mutations L234A/L235A (EU numbering) were incorporated (from 1992 paper: https://pubmed.ncbi.nlm.nih.gov/1530984/), as well as K322A (Ref https://pubmed.ncbi.nlm.nih.gov/11711607/).

For the IgG4 constructs, S228P mutations (EU numbering) were incorporated in IgG4 designs to prevent Fab arm exchange.

For the IgG2 constructs, the WT IGG2 hinge was replaced with an IGG1 hinge (DKTHTCPPCPAP) or an IGG4 hinge with S228P mutation (YGPPCPPCPAP) to maintain 2 disulfide bonds at the hinge rather than 4. It is speculated that this might decrease the possibility of proteolytic cleavage and enhance heterodimerization formation with inserted K409R/F405L mutations.

For IgG4 heterodimers, V-IGG4-A pairs with V-IGG4-B. For IgG2 heterodimers, V-IGG2-A pairs with V-IGG2-B, and V-IGG2-D pairs with V-IGG2-E.

TABLE 7
Sequences for Fc heterodimerization

| Construct | Sequence | Notes |
|---|---|---|
| WT hulgG1 | ASTKGPSVFPLAPSSKSTSGGTAALGCLVKDYFPEPVTVSWNSG | |
| CH1-CH2-CH3 | ALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFS | |
| | CSVMHEALHNHYTQKSLSLSPGK (SEQ ID NO: 51) | |
| WT hulgG4 | ASTKGPSVFPLAPCSRSTSESTAALGCLVKDYFPEPVTVSWNSG | |
| CH1-CH2-CH3 | ALTSGVHTFPAVLQSSGLYSLSSVVTVPSSSLGTKTYTCNVDHKP | |
| | SNTKVDKRVESKYGPPCPSCPAPEFLGGPSVFLFPPKPKDTLMIS | |
| | RTPEVTCVVVDVSQEDPEVQFNWYVDGVEVHNAKTKPREEQF | |
| | NSTYRVVSVLTVLHQDWLNGKEYKCKVSNKGLPSSIEKTISKAK | |
| | GQPREPQVYTLPPSQEEMTKNQVSLTCLVKGFYPSDIAVEWES | |
| | NGQPENNYKTTPPVLDSDGSFFLYSRLTVDKSRWQEGNVFSCS | |
| | VMHEALHNHYTQKSLSLSLGK (SEQ ID NO: 52) | |
| V-IGG4-A | ASTKGPSVFPLAPCSRSTSESTAALGCLVKDYFPEPVTVSWNSG | 5228P |
| | ALTSGVHTFPAVLQSSGLYSLSSVVTVPSSSLGTKTYTCNVDHKP | FALA (F234A, L235A) |
| | SNTKVDKRVESKYGPPCPPCPAPEAAGGPSVFLFPPKPKDTLMI | K322A |
| | SRTPEVTCVVVDVSQEDPEVQFNWYVDGVEVHNAKTKPREEQ | Naturally contains |
| | FNSTYRVVSVLTVLHQDWLNGKEYKCAVSNKGLPSSIEKTISKAK | F405 and R409 |
| | GQPREPQVYTLPPSQEEMTKNQVSLTCLVKGFYPSDIAVEWES | |
| | NGQPENNYKTTPPVLDSDGSFFLYSRLTVDKSRWQEGNVFSCS | |
| | VMHEALHNHYTQKSLSLSLGK (SEQ ID NO: 53) | |
| V-IGG4-A-Fc | GGGGSGGGGSGGGGSKYGPPCPPCPAPEAAGGPSVFLFPPKP | Same as SEQ ID |
| | KDTLMISRTPEVTCVVVDVSQEDPEVQFNWYVDGVEVHNAKT | NO: 53, but with |
| | KPREEQFNSTYRVVSVLTVLHQDWLNGKEYKCAVSNKGLPSSIE | linker and fc |
| | KTISKAKGQPREPQVYTLPPSQEEMTKNQVSLTCLVKGFYPSDI | only |
| | AVEWESNGQPENNYKTTPPVLDSDGSFFLYSRLTVDKSRWQE | |
| | GNVFSCSVMHEALHNHYTQKSLSLSLGK (SEQ ID NO: 54) | |
| V-IGG4-B | ASTKGPSVFPLAPCSRSTSESTAALGCLVKDYFPEPVTVSWNSG | S228P |
| | ALTSGVHTFPAVLQSSGLYSLSSVVTVPSSSLGTKTYTCNVDHKP | FALA (F234A, L235A) |
| | SNTKVDKRVESKYGPPCPPCPAPEAAGGPSVFLFPPKPKDTLMI | K322A |
| | SRTPEVTCVVVDVSQEDPEVQFNWYVDGVEVHNAKTKPREEQ | F405L, R409K |
| | FNSTYRVVSVLTVLHQDWLNGKEYKCAVSNKGLPSSIEKTISKAK | |
| | GQPREPQVYTLPPSQEEMTKNQVSLTCLVKGFYPSDIAVEWES | |
| | NGQPENNYKTTPPVLDSDGSFLLYSKLTVDKSRWQEGNVFSCS | |
| | VMHEALHNHYTQKSLSLSLGK (SEQ ID NO: 55) | |
| V-IGG4-B-Fc | GGGGSGGGGSGGGGSKYGPPCPPCPAPEAAGGPSVFLFPPKP | Same as SEQ ID |
| | KDTLMISRTPEVTCVVVDVSQEDPEVQFNWYVDGVEVHNAKT | NO: 55, but with |
| | KPREEQFNSTYRVVSVLTVLHQDWLNGKEYKCAVSNKGLPSSIE | linker and fc only |
| | KTISKAKGQPREPQVYTLPPSQEEMTKNQVSLTCLVKGFYPSDI | |
| | AVEWESNGQPENNYKTTPPVLDSDGSFLLYSKLTVDKSRWQE | |
| | GNVFSCSVMHEALHNHYTQKSLSLSLGK (SEQ ID NO: 56) | |
| WT huIgG2 | ASTKGPSVFPLAPCSRSTSESTAALGCLVKDYFPEPVTVSWNSG | |
| CH1-CH2-CH3 | ALTSGVHTFPAVLQSSGLYSLSSVVTVPSSNFGTQTYTCNVDHK | |
| | PSNTKVDKTVERKCCVECPPCPAPPVAGPSVFLFPPKPKDTLMI | |
| | SRTPEVTCVVVDVSHEDPEVQFNWYVDGVEVHNAKTKPREEQ | |
| | FNSTFRVVSVLTVVHQDWLNGKEYKCKVSNKGLPAPIEKTISKT | |
| | KGQPREPQVYTLPPSREEMTKNQVSLTCLVKGFYPSDISVEWES | |
| | NGQPENNYKTTPPMLDSDGSFFLYSKLTVDKSRWQQGNVFSC | |
| | SVMHEALHNHYTQKSLSLSPGK (SEQ ID NO: 57) | |
| V-IGG2 | ASTKGPSVFPLAPCSRSTSESTAALGCLVKDYFPEPVTVSWNSG | Contains IgG1 hinge |
| | ALTSGVHTFPAVLQSSGLYSLSSVVTVPSSNFGTQTYTCNVDHK | (DKTHTCPPCPAP) |
| | PSNTKVDKTVERKDKTHTCPPCPAPPAAGPSVFLFPPKPKDTLM | VA (V234A) |
| | ISRTPEVTCVVVDVSHEDPEVQFNWYVDGVEVHNAKTKPREE | K322A |
| | QFNSTFRVVSVLTVVHQDWLNGKEYKCAVSNKGLPAPIEKTISK | |
| | TKGQPREPQVYTLPPSREEMTKNQVSLTCLVKGFYPSDISVEWE | |
| | SNGQPENNYKTTPPMLDSDGSFFLYSKLTVDKSRWQQGNVFS | |
| | CSVMHEALHNHYTQKSLSLSPGK (SEQ ID NO: 58) | |
| V-IGG2-Fc | GGGGSGGGGSGGGGSDKTHTCPPCPAPPAAGPSVFLFPPKPK | Same as SEQ ID |
| | DTLMISRTPEVTCVVVDVSHEDPEVQFNWYVDGVEVHNAKTK | NO: 58, but with |
| | PREEQFNSTFRVVSVLTVVHQDWLNGKEYKCAVSNKGLPAPIE | linker and fc only |
| | KTISKTKGQPREPQVYTLPPSREEMTKNQVSLTCLVKGFYPSDIS | |
| | VEWESNGQPENNYKTTPPMLDSDGSFFLYSKLTVDKSRWQQG | |
| | NVFSCSVMHEALHNHYTQKSLSLSPGK (SEQ ID NO: 59) | |
| V-IGG2-A | ASTKGPSVFPLAPCSRSTSESTAALGCLVKDYFPEPVTVSWNSG | Contains IgG1 hinge |
| | ALTSGVHTFPAVLQSSGLYSLSSVVTVPSSNFGTQTYTCNVDHK | (DKTHTCPPCPAP) |
| | PSNTKVDKTVERKDKTHTCPPCPAPPAAGPSVFLFPPKPKDTLM | VA (V234A) |
| | ISRTPEVTCVVVDVSHEDPEVQFNWYVDGVEVHNAKTKPREE | K322A |
| | QFNSTFRVVSVLTVVHQDWLNGKEYKCAVSNKGLPAPIEKTISK | K409R |
| | TKGQPREPQVYTLPPSREEMTKNQVSLTCLVKGFYPSDISVEWE | |
| | SNGQPENNYKTTPPMLDSDGSFFLYSRLTVDKSRWQQGNVFS | |
| | CSVMHEALHNHYTQKSLSLSPGK (SEQ ID NO: 60) | |
| V-IGG2-A-Fc | GGGGSGGGGSGGGGSDKTHTCPPCPAPPAAGPSVFLFPPKPK | Same as SEQ ID |
| | DTLMISRTPEVTCVVVDVSHEDPEVQFNWYVDGVEVHNAKTK | NO: 60, but with |
| | PREEQFNSTFRVVSVLTVVHQDWLNGKEYKCAVSNKGLPAPIE | linker and fc only |
| | KTISKTKGQPREPQVYTLPPSREEMTKNQVSLTCLVKGFYPSDIS | |
| | VEWESNGQPENNYKTTPPMLDSDGSFFLYSRLTVDKSRWQQG | |
| | NVFSCSVMHEALHNHYTQKSLSLSPGK (SEQ ID NO: 61) | |
| V-IGG2-B | ASTKGPSVFPLAPCSRSTSESTAALGCLVKDYFPEPVTVSWNSG | Contains IgG1 hinge |
| | ALTSGVHTFPAVLQSSGLYSLSSVVTVPSSNFGTQTYTCNVDHK | (DKTHTCPPCPAP) |
| | PSNTKVDKTVERKDKTHTCPPCPAPPAAGPSVFLFPPKPKDTLM | LALA (V234A) |
| | ISRTPEVTCVVVDVSHEDPEVQFNWYVDGVEVHNAKTKPREE | K322A |
| | QFNSTFRVVSVLTVVHQDWLNGKEYKCAVSNKGLPAPIEKTISK | F405L |
| | TKGQPREPQVYTLPPSREEMTKNQVSLTCLVKGFYPSDISVEWE | |
| | SNGQPENNYKTTPPMLDSDGSFLLYSKLTVDKSRWQQGNVFS | |
| | CSVMHEALHNHYTQKSLSLSPGK (SEQ ID NO: 62) | |
| V-IGG2-B-Fc | GGGGSGGGGSGGGGSDKTHTCPPCPAPPAAGPSVFLFPPKPK | Same SEQ ID NO: 62, |
| | DTLMISRTPEVTCVVVDVSHEDPEVQFNWYVDGVEVHNAKTK | but with linker and |
| | PREEQFNSTFRVVSVLTVVHQDWLNGKEYKCAVSNKGLPAPIE | fc only |
| | KTISKTKGQPREPQVYTLPPSREEMTKNQVSLTCLVKGFYPSDIS | |
| | VEWESNGQPENNYKTTPPMLDSDGSFLLYSKLTVDKSRWQQG | |
| | NVFSCSVMHEALHNHYTQKSLSLSPGK (SEQ ID NO: 63) | |
| V-IGG2-C | ASTKGPSVFPLAPCSRSTSESTAALGCLVKDYFPEPVTVSWNSG | Contains IgG4 hinge |
| | ALTSGVHTFPAVLQSSGLYSLSSVVTVPSSNFGTQTYTCNVDHK | (YGPPCPPCPAP) with |
| | PSNTKVDKTVERKYGPPCPPCPAPPAAGPSVFLFPPKPKDTLMI | S228P |
| | SRTPEVTCVVVDVSHEDPEVQFNWYVDGVEVHNAKTKPREEQ | LALA (V234A) |
| | FNSTFRVVSVLTVVHQDWLNGKEYKCAVSNKGLPAPIEKTISKT | K322A |
| | KGQPREPQVYTLPPSREEMTKNQVSLTCLVKGFYPSDISVEWES | |
| | NGQPENNYKTTPPMLDSDGSFFLYSKLTVDKSRWQQGNVFSC | |
| | SVMHEALHNHYTQKSLSLSPGK (SEQ ID NO: 64) | |
| V-IGG2-C-Fc | GGGGSGGGGSGGGGSYGPPCPPCPAPPAAGPSVFLFPPKPKD | Same as SEQ ID NO: |
| | TLMISRTPEVTCVVVDVSHEDPEVQFNWYVDGVEVHNAKTKP | 64, but with linker |
| | REEQFNSTFRVVSVLTVVHQDWLNGKEYKCAVSNKGLPAPIEK | and fc only |
| | TISKTKGQPREPQVYTLPPSREEMTKNQVSLTCLVKGFYPSDISV | |
| | EWESNGQPENNYKTTPPMLDSDGSFFLYSKLTVDKSRWQQG | |
| | NVFSCSVMHEALHNHYTQKSLSLSPGK (SEQ ID NO: 65) | |
| V-IGG2-D | ASTKGPSVFPLAPCSRSTSESTAALGCLVKDYFPEPVTVSWNSG | Contains IgG4 hinge |
| | ALTSGVHTFPAVLQSSGLYSLSSVVTVPSSNFGTQTYTCNVDHK | (YGPPCPPCPAP) with |
| | PSNTKVDKTVERKYGPPCPPCPAPPAAGPSVFLFPPKPKDTLMI | 5228P |
| | SRTPEVTCVVVDVSHEDPEVQFNWYVDGVEVHNAKTKPREEQ | LALA (V234A) |
| | FNSTFRVVSVLTVVHQDWLNGKEYKCAVSNKGLPAPIEKTISKT | K322A |
| | KGQPREPQVYTLPPSREEMTKNQVSLTCLVKGFYPSDISVEWES | K409R |
| | NGQPENNYKTTPPMLDSDGSFFLYSRLTVDKSRWQQGNVFSC | |
| | SVMHEALHNHYTQKSLSLSPGK (SEQ ID NO: 66) | |
| V-IGG2-D-Fc | GGGGSGGGGSGGGGSYGPPCPPCPAPPAAGPSVFLFPPKPKD | Same as SEQ ID NO: |
| | TLMISRTPEVTCVVVDVSHEDPEVQFNWYVDGVEVHNAKTKP | 66, but with linker |
| | REEQFNSTFRVVSVLTVVHQDWLNGKEYKCAVSNKGLPAPIEK | and fc only |
| | TISKTKGQPREPQVYTLPPSREEMTKNQVSLTCLVKGFYPSDISV | |
| | EWESNGQPENNYKTTPPMLDSDGSFFLYSRLTVDKSRWQQG | |
| | NVFSCSVMHEALHNHYTQKSLSLSPGK (SEQ ID NO: 67) | |
| V-IGG2-E | ASTKGPSVFPLAPCSRSTSESTAALGCLVKDYFPEPVTVSWNSG | Contains IgG4 hinge |
| | ALTSGVHTFPAVLQSSGLYSLSSVVTVPSSNFGTQTYTCNVDHK | (YGPPCPPCPAP) with |
| | PSNTKVDKTVERKYGPPCPPCPAPPAAGPSVFLFPPKPKDTLMI | 5228P |
| | SRTPEVTCVVVDVSHEDPEVQFNWYVDGVEVHNAKTKPREEQ | LALA (V234A) |
| | FNSTFRVVSVLTVVHQDWLNGKEYKCAVSNKGLPAPIEKTISKT | K322A |
| | KGQPREPQVYTLPPSREEMTKNQVSLTCLVKGFYPSDISVEWES | F405L |
| | NGQPENNYKTTPPMLDSDGSFLLYSKLTVDKSRWQQGNVFSC | |
| | SVMHEALHNHYTQKSLSLSPGK (SEQ ID NO: 68) | |
| V-IGG2-E-Fc | GGGGSGGGGSGGGGSYGPPCPPCPAPPAAGPSVFLFPPKPKD | Same as SEQ ID NO: |
| | TLMISRTPEVTCVVVDVSHEDPEVQFNWYVDGVEVHNAKTKP | 68, but with linker |
| | REEQFNSTFRVVSVLTVVHQDWLNGKEYKCAVSNKGLPAPIEK | and fc only |
| | TISKTKGQPREPQVYTLPPSREEMTKNQVSLTCLVKGFYPSDISV | |
| | EWESNGQPENNYKTTPPMLDSDGSFLLYSKLTVDKSRWQQGN | |
| | VFSCSVMHEALHNHYTQKSLSLSPGK (SEQ ID NO: 69) | |

### Example 4: Design of Monospecific and 1+1 Bispecific Antibodies

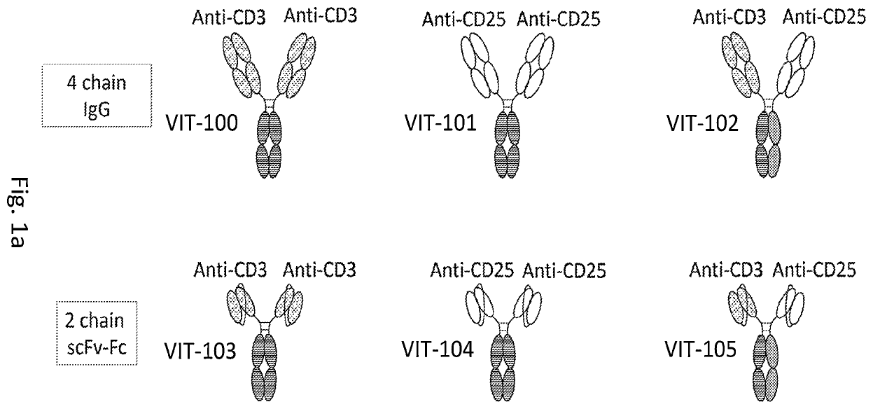

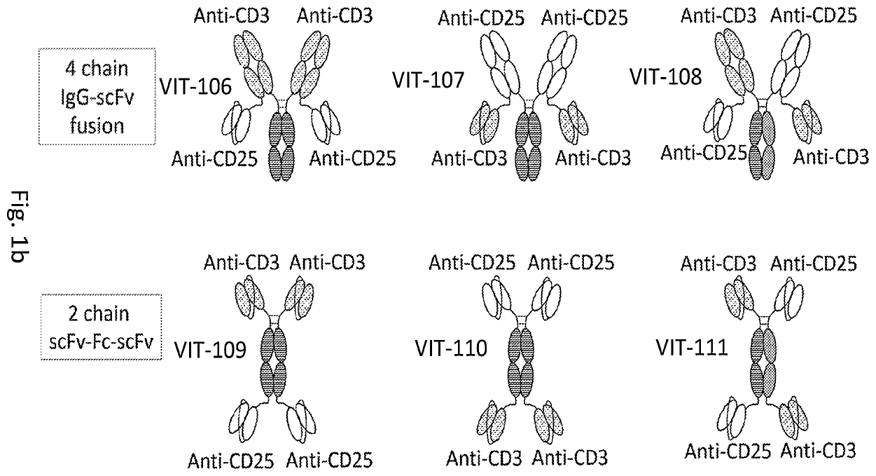

Monospecific anti-CD3, anti-CD25 and 1+1 bispecific anti-CD3×anti-CD25 antibodies were constructed based on sequences from Examples 1-3 and are presented in FIG. 1 and Table 8.

TABLE 8
Sequences of monospecific anti-CD3, anti-CD25 and 1 + 1
bispecific anti-CD3 × anti-CD25 antibodies
For the constructs in TABLE 8, the Kappa CL domain (KCL) is defined as:
RTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC (SEQ ID NO: 70)

| | Sequence of | Sequence of | Sequence of | Sequence of |
|---|---|---|---|---|
| Construct | Chain 1 | Chain 2 | Chain 3 | Chain 4 |
| VIT-100 | (VCD3-L1 or | Same as Chain 1 | (VCD3-H1 or | Same as Chain 3 |
| | VCD3-L2 or VCD3- | | VCD3-H2 or VCD3- | |
| | L3)- KCL | | H3)- | |
| | | | (V-IGG4-A or V- | |
| | | | IGG2 or V-IGG2-C) | |
| VIT-101 | (VCD25-L1 or | Same as Chain 1 | (VCD25-H1 or | Same as Chain 3 |
| | VCD25-L2)- KCL | | VCD25-H2)- | |
| | | | (V-IGG4-A or V- | |
| | | | IGG2 or V-IGG2-C) | |
| VIT-102 | (VCD3-L1 or | (VCD25-L1 or | (VCD3-H1 or | (VCD25-H1 or |
| | VCD3-L2 or VCD3- | VCD25-L2)-KCL | VCD3-H2 or VCD3- | VCD25-H2)- |
| | L3)- KCL | | H3)- | (V-IGG4-B or V- |
| | | | (V-IGG4-A or V- | IGG2-B or V-IGG2- |
| | | | IGG2-A or V-IGG2- | E) |
| | | | D) | |
| VIT-103 | (any sequence | Same as Chain 1 | | |
| | from TABLE 3)-(V- | | | |
| | IGG4-A-Fc or V- | | | |
| | IGG2-Fc or V- | | | |
| | IGG2-C-Fc) | | | |
| VIT-104 | (any sequence | Same as Chain 1 | | |
| | from TABLE 6)-(V- | | | |
| | IGG4-A-Fc or V- | | | |
| | IGG2-Fc or V- | | | |
| | IGG2-C-Fc) | | | |
| VIT-105 | (any sequence | (any sequence | | |
| | from TABLE 3)-(V- | from TABLE 6)-(V- | | |
| | IGG4-A-Fc or V- | IGG4-B-Fc or V- | | |
| | IGG2-A-Fc or V- | IGG2-B-Fc or V- | | |
| | IGG2-D-Fc) | IGG2-E-Fc) | | |

### Example 5: Design of 2+2 Bispecific Antibodies

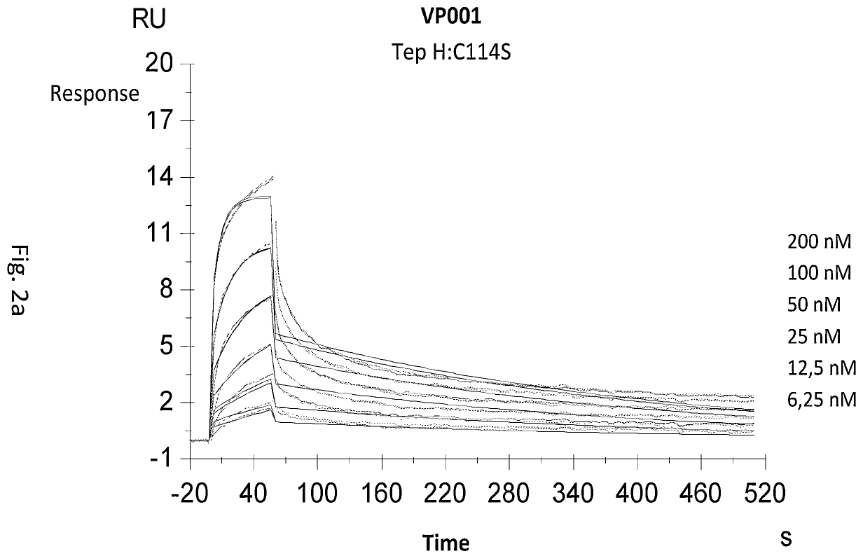

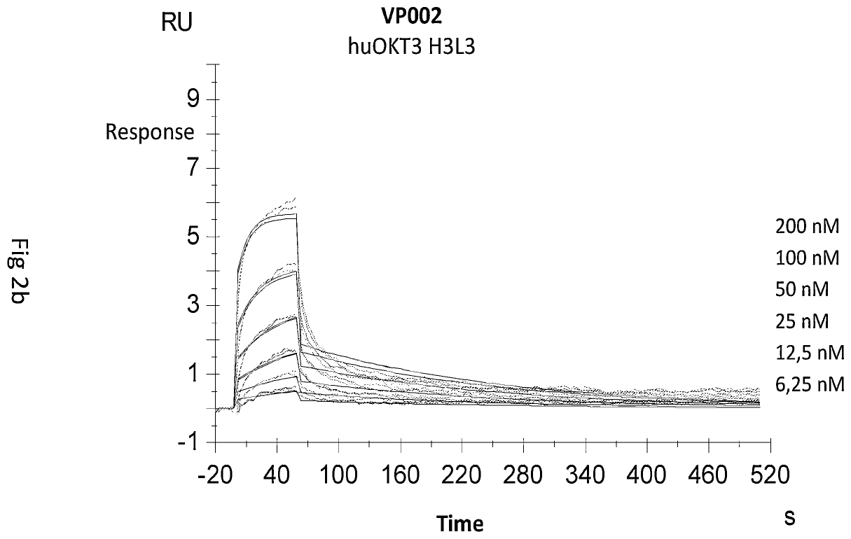

2+2 bispecific anti-CD3×anti-CD25 antibodies were constructed based on sequences from Examples 1-3 and are presented in FIG. 2 and Table 9.

For the constructs VIT-106, VIT-107 and VIT-108, the specificity on one antigen is encoded in a Fab domain, and the specificity of the second antigen is encoded by a scFv fused to the end of the light chain. It is speculated that IgG2 and IgG4 domains are ideal to make LC-scFv fusions (both as homodimers and heterodimers) because of the position of the CL-CH1 interdomain disulfide bond, which is in a different location than IgG1.

TABLE 9
Sequences of 2 + 2 bispecific anti-CD3 × anti-CD25 antibodies

| | Sequence of | Sequence of | Sequence of | Sequence of |
|---|---|---|---|---|
| Construct | Chain 1 | Chain 2 | Chain 3 | Chain 4 |
| VIT-106 | (VCD3-L1 or | Same as Chain1 | (VCD3-H1 or | Same as Chain 3 |
| | VCD3-L2 | or VCD3- | VCD3-H2 or VCD3- | |
| | L3)-KCL- | | H3)- | |
| | GGGGSGGGGSGG | | (V-IGG4-A or V- | |
| | GGS(SEQ ID NO: | | IGG2 or V-IGG2-C) | |
| | 71 x3)-(any | | | |
| | sequence from | | | |
| | TABLE 6) | | | |
| VIT-107 | (VCD25-L1 or | Same as Chain1 | (VCD25-H1 or | Same as Chain 3 |
| | VCD25-L2)-KCL- | | VCD25-H2)- | |
| | GGGGSGGGGSGG | | (V-IGG4-A or V- | |
| | GGS(SEQ ID NO: | | IGG2 or V-IGG2-C) | |
| | 71 x3)-(any | | | |
| | sequence from | | | |
| | TABLE 3) | | | |
| VIT-108 | (VCD3-L1 or | (VCD25-L1 or | (VCD3-H1 or | (VCD25-H1 or |
| | VCD3-L2 or VCD3- | VCD25-L2)-KCL- | VCD3-H2 or VCD3- | VCD25-H2)- |
| | L3)-KCL- | GGGGSGGGGSGG | H3)- | (V-IGG4-B or V- |
| | GGGGSGGGGSGG | GGS(SEQ ID NO: | (V-IGG4-A or V- | IGG2-B or V-IGG2- |
| | GGS(SEQ ID NO: | 71 x3)-(any | IGG2-A or V-IGG2- | E) |
| | 71 x3)-(any | sequence from | D) | |
| | sequence from | TABLE 3) | | |
| | TABLE 6) | | | |
| VIT-109 | (any sequence | Same as Chain 1 | | |
| | from TABLE 3)-(V- | | | |
| | IGG4-A-Fc or V- | | | |
| | IGG2-Fc or V- | | | |
| | IGG2-C-Fc)- | | | |
| | GGGGSGGGGSGG | | | |
| | GGS(SEQ ID NO: | | | |
| | 71 x3)-(any | | | |
| | sequence from | | | |
| | TABLE 6) | | | |
| VIT-110 | (any sequence | Same as Chain 1 | | |
| | from TABLE 6)-(V- | | | |
| | IGG4-A-Fc or V- | | | |
| | IGG2-Fc or V- | | | |
| | IGG2-C-Fc)- | | | |
| | GGGGSGGGGSGG | | | |
| | GGS(SEQ ID NO: | | | |
| | 71 x3)-(any | | | |
| | sequence from | | | |
| | TABLE 3) | | | |
| VIT-111 | (any sequence | (any sequence | | |
| | from TABLE 3)-(V- | from TABLE 6)-(V- | | |
| | IGG4-A-Fc or V- | IGG4-B-Fc or V- | | |
| | IGG2-A-Fc or V- | IGG2-B-Fc or V- | | |
| | IGG2-D-Fc)- | IGG2-E-Fc)- | | |
| | GGGGSGGGGSGG | GGGGSGGGGSGG | | |
| | GGS(SEQ ID NO: | GGS(SEQ ID NO: | | |
| | 71 x3)-(any | 71 x3)-(any | | |
| | sequence from | sequence from | | |
| | TABLE 6) | TABLE 3) | | |

### Example 6: Small Scale Expression and Purification of Recombinant Proteins

The 10 proteins from Table 10 were expressed and batch purified form 2.5 mL ExpiCHO cultures using Protein A/G magnetic agarose beads. The relevant formats are illustrated in FIG. 1. Expression yields and % monomeric purity are shown in Table 10.

The sequences of the proteins are disclosed in Table 10B.

TABLE 10
Small scale expression of protein constructs

| | | | | | | | % Purity |
|---|---|---|---|---|---|---|---|
| | | | | Fc | | Yield | by SEC- |
| Sample | Description | Gene 1 | Gene 2 | domain | Format | (ug) | HPLC |
| VP001 | Teplizumab H:C114S | 3P-LC | 3P-HC | IgG4 | chVIT- | 454 | 53.9 |
| | with V-IGG4-A | | | | 100 | | |
| VP002 | huOKT3 VCD3-L3H3 | 3H-LC | 3H-HC | IgG4 | huVIT- | 286 | 50.4 |
| | with V-IGG4-A | | | | 100 | | |
| VP003 | huOKT3 VCD3-L3H3 | 3H-LC-5H | 3H-HC | IgG4 | huVIT-106 | 51 | 31.3 |
| | with V-IGG4-A with LC | | | | | | |
| | fusion VCD25-H2L2- | | | | | | |
| | scFv-DS | | | | | | |
| VP004 | Teplizumab-VH-VL | 3P-SC- | | IgG4 | chVIT- | 433 | 42.8 |
| | scFv C114S DS with V- | G4 | | | 103 | | |
| | IGG4-A-Fc | | | | | | |
| VP005 | VCD3-H3L3-DS with V- | 3H-SC- | | IgG4 | huVIT- | 565 | 42.6 |
| | IGG4-A-Fc | G4 | | | 103 | | |
| VP006 | VCD3-H3L3-VIGG4-A- | 3H-SC- | | IgG4 | huVIT- | 11 | ND |
| | Fc with VCD25 H2L2 | G4-5H | | | 109 | | |
| | DS | | | | | | |
| VP007 | Basiliximab with V- | 5P-LC | 5P-HC | IgG4 | chVIT- | 195 | 52.6 |
| | IGG4-B | | | | 101 | | |
| VP008 | VCD25-L2H2 with V- | 5H-LC | 5H-HC | IgG4 | huVIT- | 186 | 54.7 |
| | IGG4-B | | | | 101 | | |
| VP009 | Basiliximab VH-VL | 5P-SC- | | IgG4 | chVIT- | 519 | 37.8 |
| | with DS with V-IGG4- | G4 | | | 104 | | |
| | B-Fc | | | | | | |
| VP010 | VCD25-H2L2-DS with | 5H-SC- | | IgG4 | huVIT- | 26 | ND |
| | V-IGG4-B-Fc | G4 | | | 104 | | |

ND = not determined due to low yield

TABLE 10B
Sequences of expressed recombinant proteins.

| Protein | | |
|---|---|---|
| Name | Description | Sequence |
| VP001 | Tep-VL | DIQMTQSPSSLSASVGDRVTITCSASSSVSYMNWYQQTPGKAPKRWIYDTSKLA |
| | with KCL | SGVPSRFSGSGSGTDYTFTISSLQPEDIATYYCQQWSSNPFTFGQGTKLQIT |
| | | RTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | (SEQ ID NO: 94) |
| | Tep-VH | QVQLVQSGGGVVQPGRSLRLSCKASGYTFTRYTMHWVRQAPGKGLEWIGYIN |
| | C114S with | PSRGYTNYNQKVKDRFTISRDNSKNTAFLQMDSLRPEDTGVYFCARYYDDHYSL |
| | V-IGG4-A | DYWGQGTPVTVSS |
| | | ASTKGPSVFPLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESKYGPPCPPCPA |
| | | PEAAGGPSVFLFPPKPKDTLMISRTPEVTCVVVDVSQEDPEVQFNWYVDGVEV |
| | | HNAKTKPREEQFNSTYRVVSVLTVLHQDWLNGKEYKCAVSNKGLPSSIEKTISKA |
| | | KGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLG |
| | | K (SEQ ID NO: 95) |
| VP002 | VCD3-L3 | EIQLTQSPATLSLSPGERATLSCRASSSVSYMNWYQQKPGQAPRRWIYDTSKLA |
| | with KCL | TGIPARFSGSGSGTDYTLTISSLEPEDAAVYYCQQWSSNPFTFGQGTKLEIK |
| | | RTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | (SEQ ID NO: 96; amino acids 1-213) |
| | VCD3-H3 | QVQLVQSGAEVKKPGASVKVSCKASGYTFTRYTMHWVRQSPGQGLEWMGYI |
| | with V- | NPSRGYTNYNQKFQGRVTMTTDKSTSTAYMELSSLRSEDTAVYYCARYYDDHYS |
| | IGG4-A | LDYWGQGTTVTVSS |
| | | ASTKGPSVFPLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESKYGPPCPPCPA |
| | | PEAAGGPSVFLFPPKPKDTLMISRTPEVTCVVVDVSQEDPEVQFNWYVDGVEV |
| | | HNAKTKPREEQFNSTYRVVSVLTVLHQDWLNGKEYKCAVSNKGLPSSIEKTISKA |
| | | KGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLG |
| | | K (SEQ ID NO: 96; amino acids 214-659) |
| VP003 | VIT-106 LC | EIQLTQSPATLSLSPGERATLSCRASSSVSYMNWYQQKPGQAPRRWIYDTSKLA |
| | VCD3-L3 - | TGIPARFSGSGSGTDYTLTISSLEPEDAAVYYCQQWSSNPFTFGQGTKLEIK |
| | KCL- linker- | RTVAAPSVFIFPPSDEQLKSGTASVVCLLNNFYPREAKVQWKVDNALQSGNSQE |
| | VCD25- | SVTEQDSKDSTYSLSSTLTLSKADYEKHKVYACEVTHQGLSSPVTKSFNRGEC |
| | H2L2-DS | GGGGGGGGSGGGGSQVQLVQSGAEVKKPGASVKVSCKASGYSFTRYWMH |
| | | WVRQPPGQCLEWMGAIYPGNSDTSYSQKFEGRVTITADTSASTAYMELSSLRS |
| | | EDTAVYYCSRDYGYYFDFWGQGTTVTVSSGGGGSGGGGSGGGGSGGGGSGG |
| | | GGSGGGGSEIVLTQSPDFQSVTPKEKVTITCRASSSRSYMQWYQQKPDQSPKRL |
| | | IYDTSKSASGVPSRFSGSGSGTDYTLTINSLEAEDAATYYCHQRSSYTFGCGTKLEI |
| | | K (SEQ ID NO: 97) |
| | VCD3-H3 | ASTKGPSVFPLAPCSRSTSESTAALGCLVKDYFPEPVTVSWNSGALTSGVHTFPA |
| | with V- | VLQSSGLYSLSSVVTVPSSSLGTKTYTCNVDHKPSNTKVDKRVESKYGPPCPPCPA |
| | IGG4-A | PEAAGGPSVFLFPPKPKDTLMISRTPEVTCVVVDVSQEDPEVQFNWYVDGVEV |
| | | HNAKTKPREEQFNSTYRVVSVLTVLHQDWLNGKEYKCAVSNKGLPSSIEKTISKA |
| | | KGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLG |
| | | K SEQ ID NO: 98) |
| VP004 | Tep-VH-VL | QVQLVQSGGGVVQPGRSLRLSCKASGYTFTRYTMHWVRQAPGKCLEWIGYIN |
| | scFv C114S | PSRGYTNYNQKVKDRFTISRDNSKNTAFLQMDSLRPEDTGVYFCARYYDDHYSL |
| | DS with V- | DYWGQGTPVTVSSGGGGSGGGGSGGGGSGGGGSGGGGSGGGGSDIQMTQ |
| | IGG4-A-Fc | SPSSLSASVGDRVTITCSASSSVSYMNWYQQTPGKAPKRWIYDTSKLASGVPSR |
| | | FSGSGSGTDYTFTISSLQPEDIATYYCQQWSSNPFTFGCGTKLQIT |
| | | GGGGSGGGGSGGGGSKYGPPCPPCPAPEAAGGPSVFLFPPKPKDTLMISRTPE |
| | | VTCVVVDVSQEDPEVQFNWYVDGVEVHNAKTKPREEQFNSTYRVVSVLTVLH |
| | | QDWLNGKEYKCAVSNKGLPSSIEKTISKAKGQPREPQVYTLPPSQEEMTKNQVS |
| | | LTCLVKGFYPSDIAVEWESNGQPENNYKTTPPVLDSDGSFFLYSRLTVDKSRWQ |
| | | EGNVFSCSVMHEALHNHYTQKSLSLSLGK (SEQ ID NO: 99) |
| VP005 | VCD3- | QVQLVQSGAEVKKPGASVKVSCKASGYTFTRYTMHWVRQSPGQCLEWMGYI |
| | H3L3-DS | NPSRGYTNYNQKFQGRVTMTTDKSTSTAYMELSSLRSEDTAVYYCARYYDDHYS |
| | with V- | LDYWGQGTTVTVSSGGGGSGGGGSGGGGSGGGGSGGGGSGGGGSEIQLTQS |
| | IGG4-A-Fc | PATLSLSPGERATLSCRASSSVSYMNWYQQKPGQAPRRWIYDTSKLATGIPARF |
| | | SGSGSGTDYTLTISSLEPEDAAVYYCQQWSSNPFTFGCGTKLEIK |
| | | GGGGSGGGGSGGGGSKYGPPCPPCPAPEAAGGPSVFLFPPKPKDTLMISRTPE |
| | | VTCVVVDVSQEDPEVQFNWYVDGVEVHNAKTKPREEQFNSTYRVVSVLTVLH |
| | | QDWLNGKEYKCAVSNKGLPSSIEKTISKAKGQPREPQVYTLPPSQEEMTKNQVS |
| | | LTCLVKGFYPSDIAVEWESNGQPENNYKTTPPVLDSDGSFFLYSRLTVDKSRWQ |
| | | EGNVFSCSVMHEALHNHYTQKSLSLSLGK (SEQ ID NO: 100) |
| VP006 | VIT-109 | QVQLVQSGAEVKKPGASVKVSCKASGYTFTRYTMHWVRQSPGQCLEWMGYI |
| | VCD3- | NPSRGYTNYNQKFQGRVTMTTDKSTSTAYMELSSLRSEDTAVYYCARYYDDHYS |
| | H3L3- | LDYWGQGTTVTVSSGGGGSGGGGSGGGGSGGGGSGGGGSGGGGSEIQLTQS |
| | VIGG4-A-Fc | PATLSLSPGERATLSCRASSSVSYMNWYQQKPGQAPRRWIYDTSKLATGIPARF |
| | with VCD25 | SGSGSGTDYTLTISSLEPEDAAVYYCQQWSSNPFTFGCGTKLEIK |
| | H2L2 DS | GGGGSGGGGSGGGGSKYGPPCPPCPAPEAAGGPSVFLFPPKPKDTLMISRTPE |
| | | VTCVVVDVSQEDPEVQFNWYVDGVEVHNAKTKPREEQFNSTYRVVSVLTVLH |
| | | QDWLNGKEYKCAVSNKGLPSSIEKTISKAKGQPREPQVYTLPPSQEEMTKNQVS |
| | | LTCLVKGFYPSDIAVEWESNGQPENNYKTTPPVLDSDGSFFLYSRLTVDKSRWQ |
| | | EGNVFSCSVMHEALHNHYTQKSLSLSLGK |
| | | GGGGSGGGGSGGGGSQVQLVQSGAEVKKPGASVKVSCKASGYSFTRYWMH |
| | | WVRQPPGQCLEWMGAIYPGNSDTSYSQKFEGRVTITADTSASTAYMELSSLRS |
| | | EDTAVYYCSRDYGYYFDFWGQGTTVTVSSGGGGSGGGGSGGGGSGGGGSGG |
| | | GGSGGGGSEIVLTQSPDFQSVTPKEKVTITCRASSSRSYMQWYQQKPDQSPKRL |
| | | IYDTSKSASGVPSRFSGSGSGTDYTLTINSLEAEDAATYYCHQRSSYTFGCGTKLEI |
| | | K (SEQ ID NO: 101) |
| VP007 | mBasil VL | QIVSTQSPAIMSASPGEKVTMTCSASSSRSYMQWYQQKPGTSPKRWIYDTSKL |
| | with KCL | ASGVPARFSGSGSGTSYSLTISSMEAEDAATYYCHQRSSYTFGGGTKLEIK |
| | | RTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | (SEQ ID NO: 102) |
| | mBasil VH | QVQLQQSGTVLARPGASVKMSCKASGYSFTRYWMHWIKQRPGQGLEWIGAIY |
| | with V- | PGNSDTSYNQKFEGKAKLTAVTSASTAYMELSSLTHEDSAVYYCSRDYGYYFDF |
| | IGG4-B | WGQGTTLTVSS |
| | | ASTKGPSVFPLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESKYGPPCPPCPA |
| | | PEAAGGPSVFLFPPKPKDTLMISRTPEVTCVVVDVSQEDPEVQFNWYVDGVEV |
| | | HNAKTKPREEQFNSTYRVVSVLTVLHQDWLNGKEYKCAVSNKGLPSSIEKTISKA |
| | | KGQPREPQVYTLPPSQEEMTKNQVSLTCLVKGFYPSDIAVEWESNGQPENNYK |
| | | TTPPVLDSDGSFLLYSKLTVDKSRWQEGNVFSCSVMHEALHNHYTQKSLSLSLG |
| | | K (SEQ ID NO: 103) |
| VP008 | VCD25-L2 | EIVLTQSPDFQSVTPKEKVTITCRASSSRSYMQWYQQKPDQSPKRLIYDTSKSAS |
| | with KCL | GVPSRFSGSGSGTDYTLTINSLEAEDAATYYCHQRSSYTFGQGTKLEIK |
| | | RTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | (SEQ ID NO: 104) |
| | VCD25-H2 | QVQLVQSGAEVKKPGASVKVSCKASGYSFTRYWMHWVRQPPGQRLEWMGA |
| | with V- | IYPGNSDTSYSQKFEGRVTITADTSASTAYMELSSLRSEDTAVYYCSRDYGYYFDF |
| | IGG4-B | WGQGTTVTVSS |
| | | ASTKGPSVFPLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESKYGPPCPPCPA |
| | | PEAAGGPSVFLFPPKPKDTLMISRTPEVTCVVVDVSQEDPEVQFNWYVDGVEV |
| | | HNAKTKPREEQFNSTYRVVSVLTVLHQDWLNGKEYKCAVSNKGLPSSIEKTISKA |
| | | KGQPREPQVYTLPPSQEEMTKNQVSLTCLVKGFYPSDIAVEWESNGQPENNYK |
| | | TTPPVLDSDGSFLLYSKLTVDKSRWQEGNVFSCSVMHEALHNHYTQKSLSLSLG |
| | | K (SEQ ID NO: 105) |
| VP009 | Bas VH-VL | QVQLVQSGAEVKKPGASVKVSCKASGYSFTRYWMHWVRQPPGQCLEWMGA |
| | with DS | IYPGNSDTSYSQKFEGRVTITADTSASTAYMELSSLRSEDTAVYYCSRDYGYYFDF |
| | with V- | WGQGTTVTVSSGGGGSGGGGSGGGGSGGGGSGGGGSGGGGSEIVLTQSPDF |
| | IGG4-B-Fc | QSVTPKEKVTITCRASSSRSYMQWYQQKPDQSPKRLIYDTSKSASGVPSRFSGSG |
| | | SGTDYTLTINSLEAEDAATYYCHQRSSYTFGCGTKLEIKGGGGSGGGGSGGGGS |
| | | KYGPPCPPCPAPEAAGGPSVFLFPPKPKDTLMISRTPEVTCVVVDVSQEDPEVQF |
| | | NWYVDGVEVHNAKTKPREEQFNSTYRVVSVLTVLHQDWLNGKEYKCAVSNKG |
| | | LPSSIEKTISKAKGQPREPQVYTLPPSQEEMTKNQVSLTCLVKGFYPSDIAVEWES |
| | | NGQPENNYKTTPPVLDSDGSFLLYSKLTVDKSRWQEGNVFSCSVMHEALHNHY |
| | | TQKSLSLSLGK (SEQ ID NO: 106) |
| VP010 | VCD25- | QVQLVQSGAEVKKPGASVKVSCKASGYSFTRYWMHWVRQPPGQCLEWMGA |
| | H2L2-DS | IYPGNSDTSYSQKFEGRVTITADTSASTAYMELSSLRSEDTAVYYCSRDYGYYFDF |
| | with V- | WGQGTTVTVSSGGGGSGGGGSGGGGSGGGGSGGGGSGGGGSEIVLTQSPDF |
| | IGG4-B-Fc | QSVTPKEKVTITCRASSSRSYMQWYQQKPDQSPKRLIYDTSKSASGVPSRFSGSG |
| | | SGTDYTLTINSLEAEDAATYYCHQRSSYTFGCGTKLEIK |
| | | GGGGSGGGGSGGGGSKYGPPCPPCPAPEAAGGPSVFLFPPKPKDTLMISRTPE |
| | | VTCVVVDVSQEDPEVQFNWYVDGVEVHNAKTKPREEQFNSTYRVVSVLTVLH |
| | | QDWLNGKEYKCAVSNKGLPSSIEKTISKAKGQPREPQVYTLPPSQEEMTKNQVS |
| | | LTCLVKGFYPSDIAVEWESNGQPENNYKTTPPVLDSDGSFLLYSKLTVDKSRWQE |
| | | GNVFSCSVMHEALHNHYTQKSLSLSLGK (SEQ ID NO: 107) |
| VP100 | ch7G7 VL | QIVLSQSPAILSASPGERVTMTCRASSSVSFMHWLQQKPGSSPKPWIYATSNLA |
| | with KCL | SGVSARFSGSGSGTSYSLTITRVEAEDAATYYCQQWSSNPPAFGGGTKLEIK |
| | | RTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | (SEQ ID NO: 108) |
| | ch7G7 with | EVQLVESGGDLVQPRGSLKLSCAASGFTFSSYGMSWVRQTPDKRLELVATINGY |
| | V-IGG4-B- | GDTTYYPDSVKGRFTISRDNAKNTLYLQMSSLKSEDTAMYFCARDRDYGNSYYY |
| | Fc | ALDYWGQGTSVTVSSASTKGPSVFPLAPCSRSTSESTAALGCLVKDYFPEPVTVS |
| | | WNSGALTSGVHTFPAVLQSSGLYSLSSVVTVPSSSLGTKTYTCNVDHKPSNTKVD |
| | | KRVESKYGPPCPPCPAPEAAGGPSVFLFPPKPKDTLMISRTPEVTCVVVDVSQED |
| | | PEVQFNWYVDGVEVHNAKTKPREEQFNSTYRVVSVLTVLHQDWLNGKEYKCA |
| | | VSNKGLPSSIEKTISKAKGQPREPQVYTLPPSQEEMTKNQVSLTCLVKGFYPSDIA |
| | | VEWESNGQPENNYKTTPPVLDSDGSFLLYSKLTVDKSRWQEGNVFSCSVMHEA |
| | | LHNHYTQKSLSLSLGK (SEQ ID NO: 109) |
| VP101 | hu7G7 VL | EIVLTQSPATLSLSPGERATLSCRASSSVSFMHWLQQKPGQAPKPLIYATSNLAS |
| | with KCL | GIPARFSGSGSGTDYTLTISSLEPEDFAVYYCQQWSSNPPAFGGGTKVEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC (SEQ ID |
| | | NO: 110) |
| | hu7G7 with | EVQLVESGGGLVQPGGSLRLSCAASGFTFSSYGMSWVRQAPGKRLELVATINGY |
| | V-IGG4-B- | GDTTYYPDSVKGRFTISRDNAKNTLYLQMNSLRAEDTAVYYCARDRDYGNSYYY |
| | Fc | ALDYWGQGTLVTVSSASTKGPSVFPLAPCSRSTSESTAALGCLVKDYFPEPVTVS |
| | | WNSGALTSGVHTFPAVLQSSGLYSLSSVVTVPSSSLGTKTYTCNVDHKPSNTKVD |
| | | KRVESKYGPPCPPCPAPEAAGGPSVFLFPPKPKDTLMISRTPEVTCVVVDVSQED |
| | | PEVQFNWYVDGVEVHNAKTKPREEQFNSTYRVVSVLTVLHQDWLNGKEYKCA |
| | | VSNKGLPSSIEKTISKAKGQPREPQVYTLPPSQEEMTKNQVSLTCLVKGFYPSDIA |
| | | VEWESNGQPENNYKTTPPVLDSDGSFLLYSKLTVDKSRWQEGNVFSCSVMHEA |
| | | LHNHYTQKSLSLSLGK (SEQ ID NO: 111) |
| VP102A | Tep-VH-VL | QVQLVQSGGGVVQPGRSLRLSCKASGYTFTRYTMHWVRQAPGKCLEWIGYIN |
| | scFv C114S | PSRGYTNYNQKVKDRFTISRDNSKNTAFLQMDSLRPEDTGVYFCARYYDDHYSL |
| | DS and | DYWGQGTPVTVSSGGGGSGGGGSGGGGSGGGGSGGGGSGGGGSDIQMTQ |
| | hu7G7 scFv | SPSSLSASVGDRVTITCSASSSVSYMNWYQQTPGKAPKRWIYDTSKLASGVPSR |
| | with V- | FSGSGSGTDYTFTISSLQPEDIATYYCQQWSSNPFTFGCGTKLQITGGGGSGGG |
| | IGG4-A-Fc | GSGGGGSEVOLVESGGGLVQPGGSLRLSCAASGFTFSSYGMSWVRQAPGKCLE |
| | | LVATINGYGDTTYYPDSVKGRFTISRDNAKNTLYLQMNSLRAEDTAVYYCARDR |
| | | DYGNSYYYALDYWGQGTLVTVSSGGGGSGGGGSGGGGSGGGGSGGGGSGG |
| | | GGSEIVLTQSPATLSLSPGERATLSCRASSSVSFMHWLQQKPGQAPKPLIYATSN |
| | | LASGIPARFSGSGSGTDYTLTISSLEPEDFAVYYCQQWSSNPPAFGCGTKVEIKGG |
| | | GGSGGGGSGGGGSKYGPPCPPCPAPEAAGGPSVFLFPPKPKDTLMISRTPEVTC |
| | | VVVDVSQEDPEVQFNWYVDGVEVHNAKTKPREEQFNSTYRVVSVLTVLHQD |
| | | WLNGKEYKCAVSNKGLPSSIEKTISKAKGQPREPQVYTLPPSQEEMTKNQVSLTC |
| | | LVKGFYPSDIAVEWESNGQPENNYKTTPPVLDSDGSFFLYSRLTVDKSRWQEGN |
| | | VFSCSVMHEALHNHYTQKSLSLSLGK (SEQ ID NO: 112) |
| VP102B | Tep-VH-VL | QVQLVQSGGGVVQPGRSLRLSCKASGYTFTRYTMHWVRQAPGKCLEWIGYIN |
| | scFv C114S | PSRGYTNYNQKVKDRFTISRDNSKNTAFLQMDSLRPEDTGVYFCARYYDDHYSL |
| | DS with V- | DYWGQGTPVTVSSGGGGSGGGGSGGGGSGGGGSGGGGSGGGGSDIQMTQ |
| | IGG4-A-Fc | SPSSLSASVGDRVTITCSASSSVSYMNWYQQTPGKAPKRWIYDTSKLASGVPSR |
| | and and | FSGSGSGTDYTFTISSLQPEDIATYYCQQWSSNPFTFGCGTKLQITGGGGSGGG |
| | hu7G7 scFv | GSGGGGSKYGPPCPPCPAPEAAGGPSVFLFPPKPKDTLMISRTPEVTCVVVDVS |
| | | QEDPEVQFNWYVDGVEVHNAKTKPREEQFNSTYRVVSVLTVLHQDWLNGKEY |
| | | KCAVSNKGLPSSIEKTISKAKGQPREPQVYTLPPSQEEMTKNQVSLTCLVKGFYPS |
| | | DIAVEWESNGQPENNYKTTPPVLDSDGSFFLYSRLTVDKSRWQEGNVFSCSVM |
| | | HEALHNHYTQKSLSLSLGKGGGGSGGGGSGGGGSEVOLVESGGGLVQPGGSL |
| | | RLSCAASGFTFSSYGMSWVRQAPGKCLELVATINGYGDTTYYPDSVKGRFTISRD |
| | | NAKNTLYLQMNSLRAEDTAVYYCARDRDYGNSYYYALDYWGQGTLVTVSSGG |
| | | GGSGGGGSGGGGSGGGGSGGGGSGGGGSEIVLTQSPATLSLSPGERATLSCRA |
| | | SSSVSFMHWLQQKPGQAPKPLIYATSNLASGIPARFSGSGSGTDYTLTISSLEPED |
| | | FAVYYCQQWSSNPPAFGCGTKVEIK (SEQ ID NO: 113) |
| VP103 | Alpaca | QVQLQESGGGLVQAGGSLTLSCAASGFTLDDYAIGWFRQAPGKEREGVSCIRRS |
| | Anti-CD3 | DGHTYSADSVKGRFTISSDNAKNTVYLQMNNLKPEDTAVYYCAAGTGWGRYFY |
| | nanobody | AGMDYWGKGTQVTVSSGGGGSGGGGSGGGGSKYGPPCPPCPAPEAAGGPS |
| | F10 with | VFLFPPKPKDTLMISRTPEVTCVVVDVSQEDPEVQFNWYVDGVEVHNAKTKPR |
| | with V- | EEQFNSTYRVVSVLTVLHQDWLNGKEYKCAVSNKGLPSSIEKTISKAKGQPREPQ |
| | IGG4-A-Fc | VYTLPPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK (SEQ ID |
| | | NO: 114) |
| VP104 | humanized | EVQLLESGGGLVQPGGSLRLSCAASGFTLDDYAIGWVRQAPGKGREGVSAIRRS |
| | Anti-CD3 | DGHTYYADSVKGRFTISSDNSKNTVYLQMNSLRAEDTAVYYCAAGTGWGRYFY |
| | nanobody | AGMDYWGQGTLVTVSSGGGGSGGGGSGGGGSKYGPPCPPCPAPEAAGGPSV |
| | F10 with | FLFPPKPKDTLMISRTPEVTCVVVDVSQEDPEVQFNWYVDGVEVHNAKTKPRE |
| | free Cys | EQFNSTYRVVSVLTVLHQDWLNGKEYKCAVSNKGLPSSIEKTISKAKGQPREPQV |
| | removed | YTLPPSQEEMTKNQVSLTCLVKGFYPSDIAVEWESNGQPENNYKTTPPVLDSDG |
| | and with V- | SFFLYSRLTVDKSRWQEGNVFSCSVMHEALHNHYTQKSLSLSLGK (SEQ ID NO: |
| | IGG4-A-Fc | 115) |
| VP105 | humanized | EVQLLESGGGLVQPGGSLRLSCAASGFTLDDYAIGWVRQAPGKGREGVSCIRRS |
| | Anti-CD3 | DGHTYYADSVKGRFTISSDNSKNTVYLQMNSLRAEDTAVYYCAAGTGWGRYFY |
| | nanobody | AGMDYWGQGTLVTVSSGGGGSGGGGSGGGGSKYGPPCPPCPAPEAAGGPSV |
| | F10 | FLFPPKPKDTLMISRTPEVTCVVVDVSQEDPEVQFNWYVDGVEVHNAKTKPRE |
| | (contains | EQFNSTYRVVSVLTVLHQDWLNGKEYKCAVSNKGLPSSIEKTISKAKGQPREPQV |
| | free Cys) | YTLPPSQEEMTKNQVSLTCLVKGFYPSDIAVEWESNGQPENNYKTTPPVLDSDG |
| | with V- | SFFLYSRLTVDKSRWQEGNVFSCSVMHEALHNHYTQKSLSLSLGK (SEQ ID NO: |
| | IGG4-A-Fc | 116) |
| VP106 | humanized | EVQLLESGGGLVQPGGSLRLSCAASGFTLDDYAIGWVRQAPGKGREGVSAIRRS |
| | Anti-CD3 | DGHTYYADSVKGRFTISSDNSKNTVYLQMNSLRAEDTAVYYCAAGTGWGRYFY |
| | nanobody | AGMDYWGQGTLVTVSSGGGGSGGGGSGGGGSEVOLVESGGGLVQPGGSLRL |
| | F10 | SCAASGFTFSSYGMSWVRQAPGKCLELVATINGYGDTTYYPDSVKGRFTISRDN |
| | (contains | AKNTLYLQMNSLRAEDTAVYYCARDRDYGNSYYYALDYWGQGTLVTVSSGGG |
| | free Cys) | GSGGGGSGGGGSGGGGSGGGGSGGGGSEIVLTQSPATLSLSPGERATLSCRAS |
| | and hu7G7 | SSVSFMHWLQQKPGQAPKPLIYATSNLASGIPARFSGSGSGTDYTLTISSLEPEDF |
| | scFv with | AVYYCQQWSSNPPAFGCGTKVEIKGGGGSGGGGSGGGGSKYGPPCPPCPAPE |
| | V-IGG4-A- | AAGGPSVFLFPPKPKDTLMISRTPEVTCVVVDVSQEDPEVQFNWYVDGVEVHN |
| | Fc | AKTKPREEQFNSTYRVVSVLTVLHQDWLNGKEYKCAVSNKGLPSSIEKTISKAKG |
| | | QPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| | | (SEQ ID NO: 117) |

### Example 7: Binding Affinity Measurements for VP001, VP002, VP007 and VP008

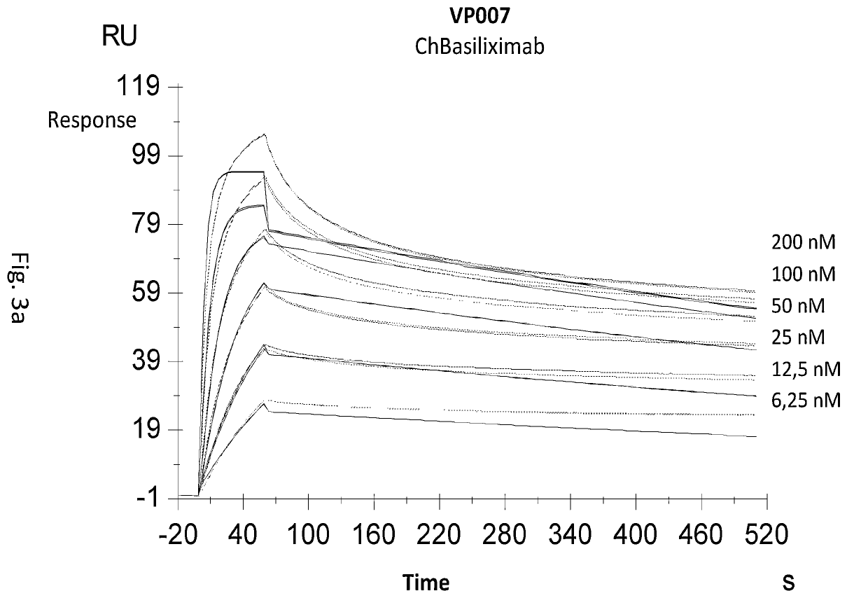

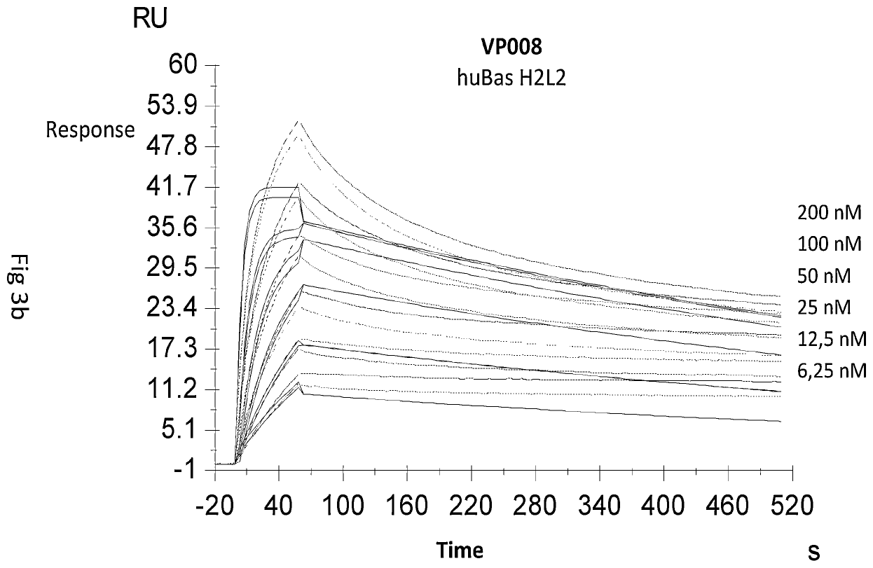

The relative affinities of VP001, VP002, VP007 and VP008 for recombinant human CD3de (Delta epsilon fusion) and recombinant human CD25 were assessed by SPR on a Biacore T200 (GE Healthcare). FIGS. 2a and 2b shows the SPR sensorgrams for VP001 and VP002 respectively to human CD3de immobilized onto the surface of CM5 sensor chip. VP001 (Teplizumab H:C114S) had a KD of 5.2 nM, while VP002 (huOKT3 H3L3) had a KD of 12.4 nM. FIGS. 3a and 3b shows the SPR sensorgrams for VP007 and VP008 respectively to human CD25 immobilized onto the surface of CM5 sensor chip. VP007 (chimeric Basiliximab) had a KD of 0.8 nM, while VP008 (huBasiliximab H2L2) had a KD of 1.2 nM.

### Example 8: Larger Scale Expression and Purification of VP001, VP002 and VP008

Larger scale preps were done in ExpiCHO cells for VP001 (100 mL), VP002 (250 mL), VP008 (250 mL). The products were purified by MabSelect SuRe protein A resin column chromatography. VP001 and VP008 were purified with by SEC-HPLC. Expression yields and % monomeric purity are shown in Table 11.

TABLE 11
Larger scale expression of protein constructs

| | | | Initial % | % Purity after |
|---|---|---|---|---|
| | | Yield | Purity by | preparatory |
| Protein Name | Format | (mg) | SEC-HPLC | SEC-HPLC |
| VP001 | chVIT-100 | 25.0 | 74.5 | 97.1 |
| VP002 | huVIT-100 | 24.3 | 93.4 | ND |
| VP008 | huVIT-101 | 23.9 | 74.8 | 99.0 |

*ND = Not done

### Example 9: Preparation of Bispecific Heterodimer Molecules

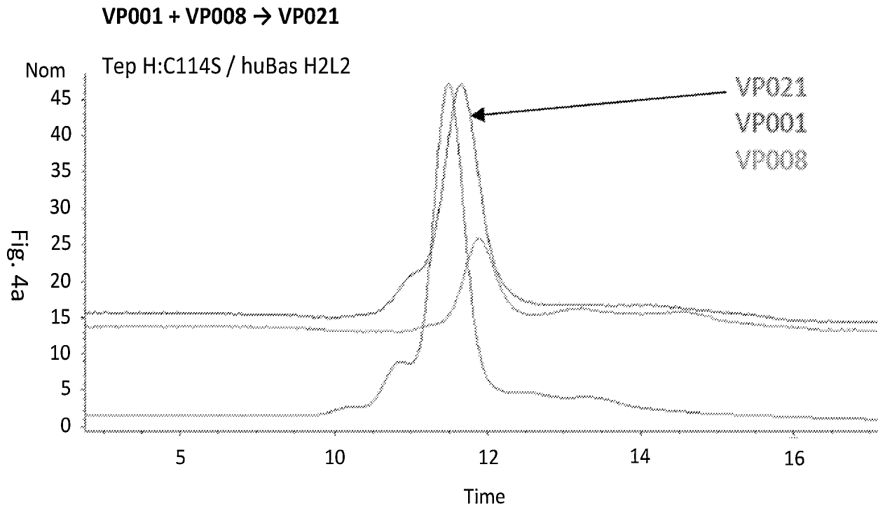

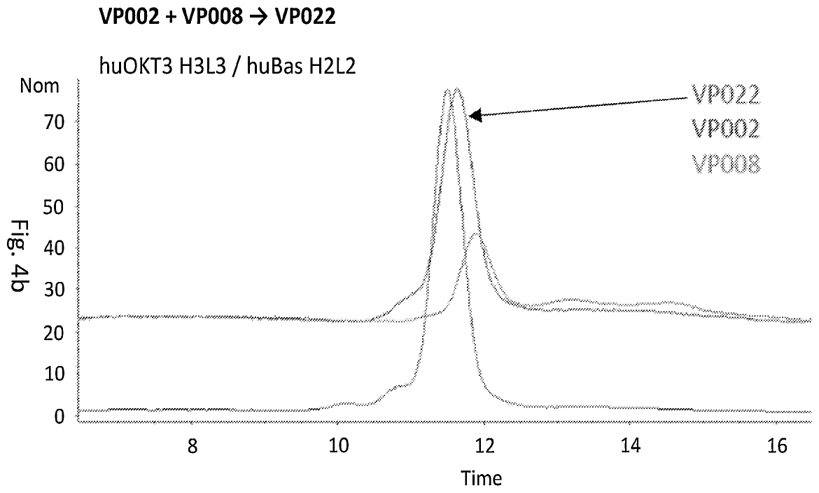

CD3×CD25 Bispecific heterodimer products VP021 and VP022 (see FIGS. 4a and 4b) were generated by mixing the parent homodimer molecules in equimolar amount under mild reducing agent 2-MEA (2-Mercaptoethylamine) for 5 h at 31 deg C. and then removing the 2-MEA to allow re-oxidation of the hinge disulfide bonds, as described by Labrijn et al. (https://www.nature.com/articles/nprot.2014.169) VP021 was formed by mixing VP001 and VP008. VP022 was formed by mixing VP002 and VP008. Hydrophobic interaction chromatography (HIC) profiles shown in FIGS. 4a and 4b show that VP021 and VP022 efficiently formed heterodimers. The purity by SEC-HPLC is shown in Table 12.

TABLE 12
Properties of CD3 × CD25 bispecific heterodimers VP021 and VP022

| | | % Purity by |
|---|---|---|
| Protein Name | Format | SEC-HPLC |
| VP021 | VIT-102 | 91.6 |
| VP022 | VIT-102 | 94.5 |

### Example 10: Treg Suppression Assay

A Treg suppression assay was performed with autologous CD8+ T Cells and regulatory T cells (Tregs; CD4+CD25+) from a normal healthy human donor in the presence of test article treatment. The objective was to see if the test articles could enhance the ability of Tregs to inhibit the proliferation of CD8+ cytotoxic T cells. Tregs were incubated with three concentrations of VP001, VP002, VP008, VP021, VP022, and IgG4 isotype control antibody (0.1, 1, and 10 μg/mL) and control test articles (2 ng/mL FK506, 12.5 ng/mL PMA/50 ng/mL lonomycin, and 10 U/mL IL-2), or left untreated in triplicate.

CFSE-labeled CD8+ T Cells and Tregs were cocultured at a 3:1 ratio with human T cell activation beads (1:2 bead to CD8+ T cell ratio) for 96 hours at 37 degrees C., 5% CO2.

Cells were harvested and stained with fluorescently labeled anti-CD4 antibodies to identify the CD4⁺ regulatory T cells. Diluted CFSE signal was gated on (excluding the CFSE-unlabeled signal) within the CD4⁻ population to ascertain the percentage of CD8⁺ T cells that had proliferated.

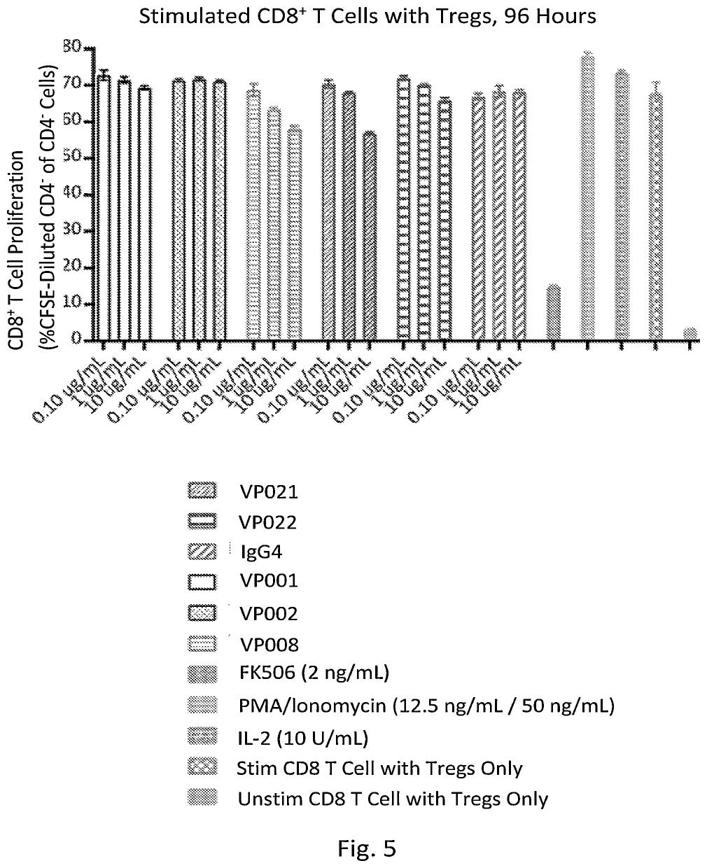

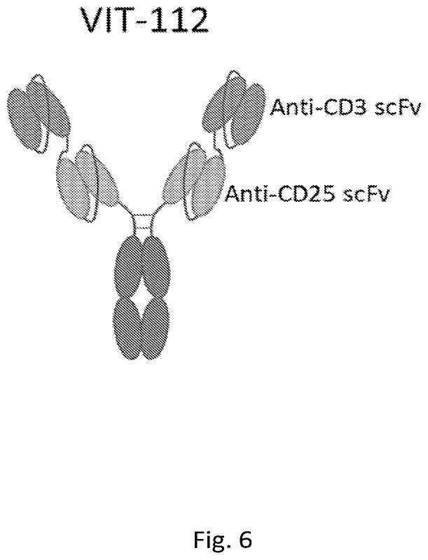

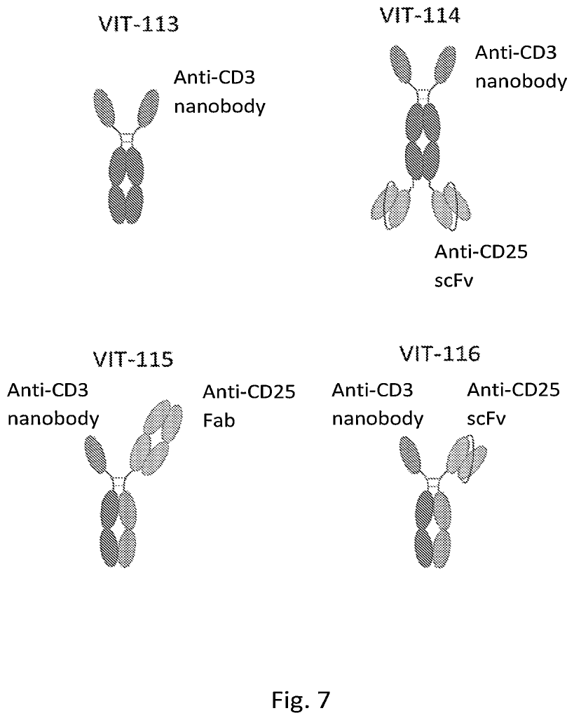

FIG. 5 shows CD8+ T cells proliferation without the addition of Tregs and in the cocultures with Tregs in response to treatment with different compounds. VP008 and VP021 treatment led to a decrease in CD8⁺ T cell proliferation in the presence of Tregs in a dose-dependent manner. VP022 also showed some modest decrease in CD8⁺ T cell proliferation in the presence of Tregs. The same trends were not observed when the test articles were added to CD8+ T cell in the absence of Treg cells.

### Example 11: Humanization of Anti-CD25 Murine mAb 7G7

The murine mAbs 7G7 (Rubin et al., (https://pubmed.ncbi.nlm.nih.gov/2408992/)) has been shown to bind CD25 at an epitope that does not block IL-2, unlike basiliximab.

A proposed sequence for a humanized anti-CD25 murine mAb 7G7 is hereby provided as follows.

The sequence of anti-CD25 murine mAb 7G7 is shown below where IMGT CDR sequences are underlined. The CDR sequences of the VL domain are designated SEQ ID NO: 125-127, and the CDR sequences of the VH domain are designated SEQ ID NO: 128-130.

VL:
(SEQ ID NO: 118)
QIVLSQSPAILSASPGERVTMTCRASSSVSFMHWLQQKPGSSPKPWIYAT
SNLASGVSARFSGSGSGTSYSLTITRVEAEDAATYYCQQWSSNPPAFGGG
TKLEIK
VH:
(SEQ ID NO: 119)
EVQLVESGGDLVQPRGSLKLSCAASGFTFSSYGMSWVRQTPDKRLELVAT
INGYGDTTYYPDSVKGRFTISRDNAKNTLYLQMSSLKSEDTAMYFCARDR
DYGNSYYYALDYWGQGTSVTVSS

A molecular model of the murine Fv of mAb 7G7 was generated using Discovery Studio modeling software (Dassault Systemes) and then utilized to rationally engineer humanizing mutations based on identity to human germline sequence and how these mutations could possibly affect antigen binding and internal protein stability. The humanized 7G7 sequence is shown below.

VL:
(SEQ ID NO: 120)
EIVLTQSPATLSLSPGERATLSCRASSSVSFMHWLQQKPGQAPKPLIYAT
SNLASGIPARFSGSGSGTDYTLTISSLEPEDFAVYYCQQWSSNPPAFGGG
TKVEIK

Sequence 120 has 83.2% identity to human germline sequence IGKV3-11*01 and 100% identity to IGKJ4*01.

VH:
(SEQ ID NO: 121)
EVQLVESGGGLVQPGGSLRLSCAASGFTFSSYGMSWVRQAPGKRLELVAT
INGYGDTTYYPDSVKGRFTISRDNAKNTLYLQMNSLRAEDTAVYYCARDR
DYGNSYYYALDYWGQGTLVTVSS

Sequence 121 has 87.8% identity to human germline sequence IGHV3-23*04 and 92.9% identity to IGHJ4*01.

### Example 12: Humanization of Anti-CD3 Alpaca VHH Nanobody F10

A proposed sequence for a humanized anti-CD3 VHH nanobody F10 is hereby provided as follows.

The sequence of anti-CD3 alpaca VHH F10 is shown below where IMGT CDR sequences are underlined. The CDR sequences are designated SEQ ID NO: 131-133.

Alpaca F10 VHH
(SEQ ID NO: 122)
QVQLQESGGGLVQAGGSLTLSCAASGFTLDDYAIGWFRQAPGKEREGVSC
IRRSDGHTYSADSVKGRFTISSDNAKNTVYLQMNNLKPEDTAVYYCAAGT
GWGRYFYAGMDYWGKGTQVTVSS

A molecular model of alpaca VHH F10 was generated using Discovery Studio modeling software (Dassault Systemes) and then utilized to rationally engineer humanizing mutations based on identity to human germline sequence and how these mutations could possibly affect antigen binding and internal protein stability. The humanized F10 VHH is shown below.

Humanized F10 VHH
(SEQ ID NO: 123)
EVQLLESGGGLVQPGGSLRLSCAASGFTLDDYAIGWVRQAPGKGREGVSC
IRRSDGHTYYADSVKGRFTISSDNSKNTVYLQMNSLRAEDTAVYYCAAGT
GWGRYFYAGMDYWGQGTLVTVSS

Sequence 123 has 85.6% identity to human germline sequence IGHV3-23*01 and 100% identity to IGHJ4*01.

A second humanized construct huF10A was generated to replace a free unpaired Cys at position 55 (shown in bold) with an Ala found in the closest human germline sequence. This was done to prevent aggregation common to protein with unpaired Cys residues.

Humanized F10A VHH (C55A)
(SEQ ID NO: 124)
EVQLLESGGGLVQPGGSLRLSCAASGFTLDDYAIGWVRQAPGKGREGVSA
IRRSDGHTYYADSVKGRFTISSDNSKNTVYLQMNSLRAEDTAVYYCAAGT
GWGRYFYAGMDYWGQGTLVTVSS

Sequence 124 has 86.6% identity to human germline sequence IGHV3-23*01 and 100% identity to IGHJ4*01.

### Example 13: Binding Affinity Measurements for VP100 and VP101

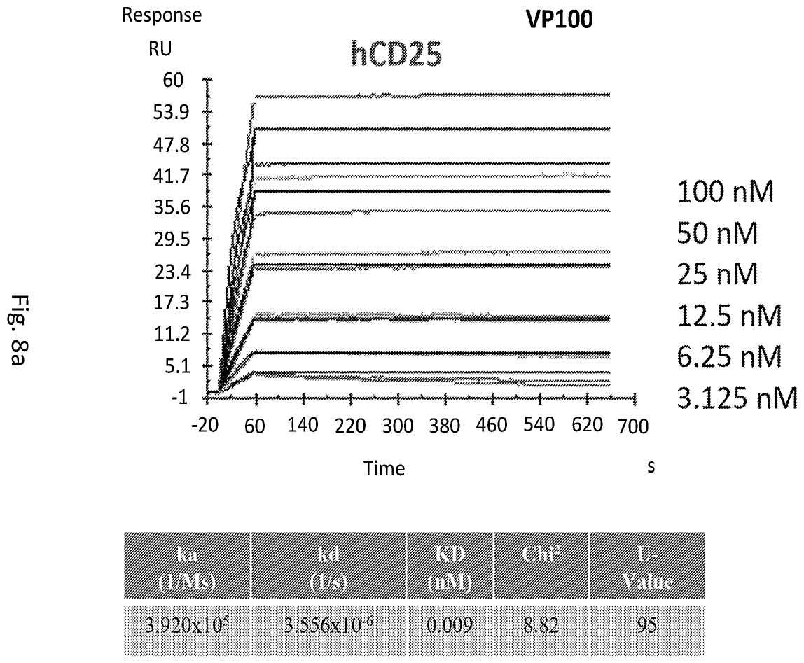

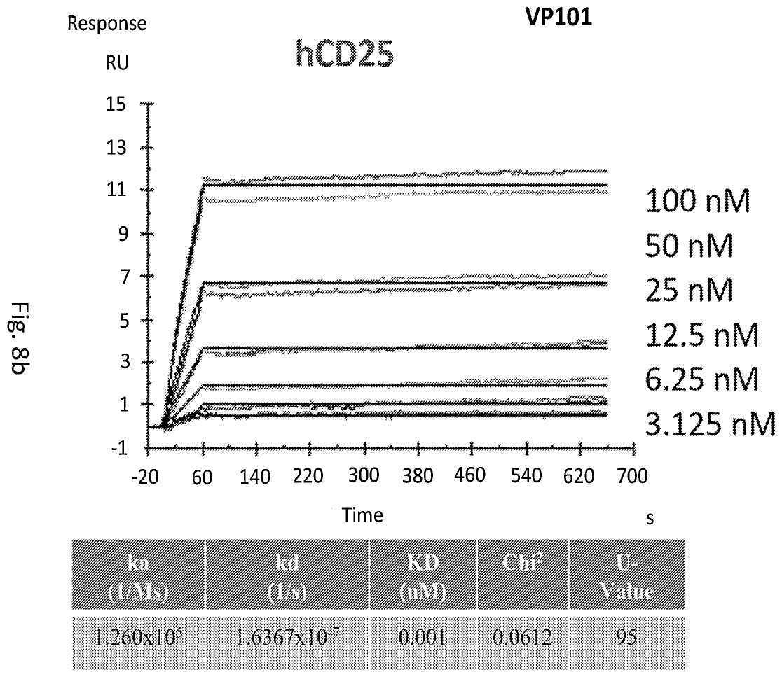

The binding affinities of VP100 (ch7G7-IgG4) and VP101 (hu7G7-IgG4) for recombinant human CD25 were assessed by SPR on a Biacore T200 (GE Healthcare). FIGS. 8a and 8b show the SPR sensorgrams for VP100 and VP101 to human CD25 immobilized onto the surface of CM5 sensor chip. The data was fit with a 1:1 fitting model. The KD of VP100 was 9 pM. VP101 unexpectedly had higher affinity to CD25 than VP100, with an apparent KD1 of <1 pM.

### Example 14: Binding Affinity Measurements for VP103 and VP104

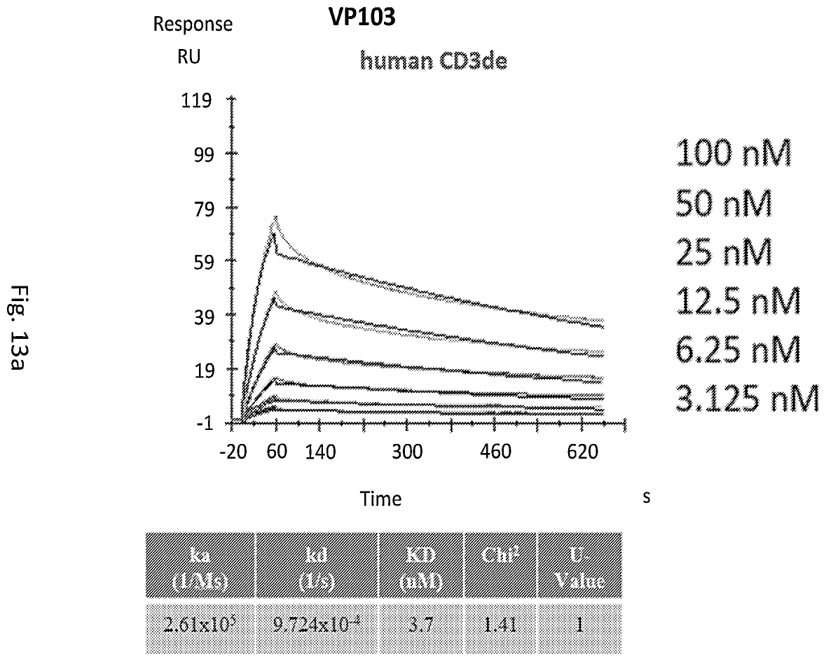

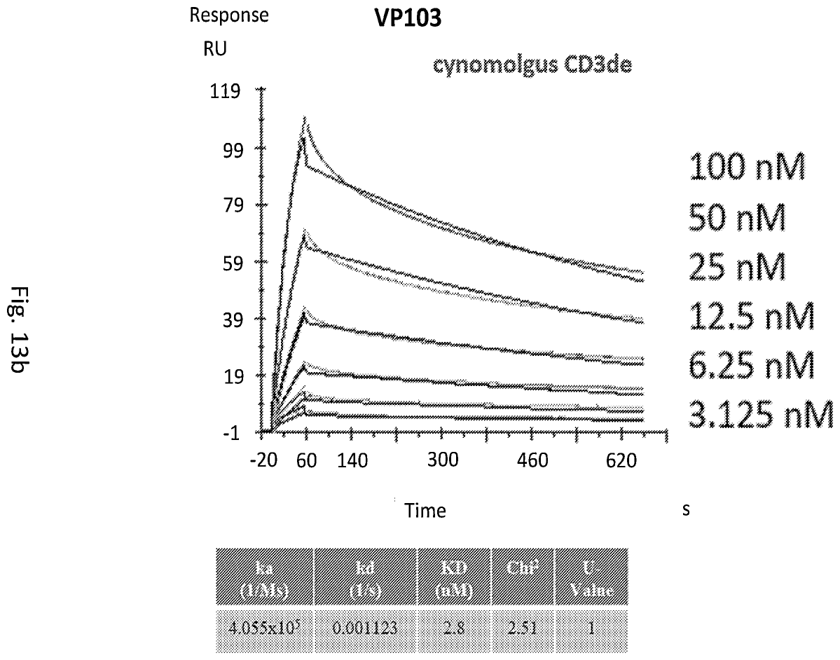

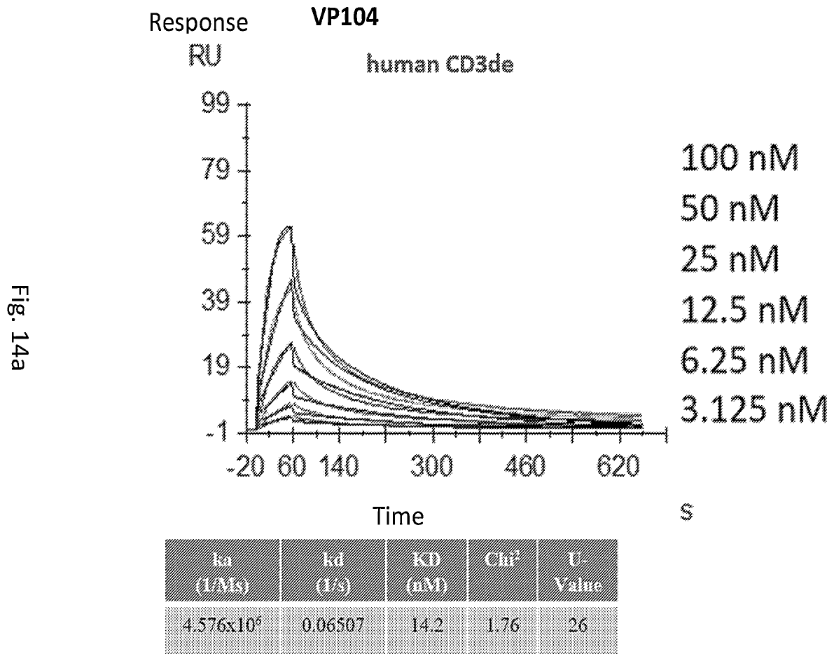

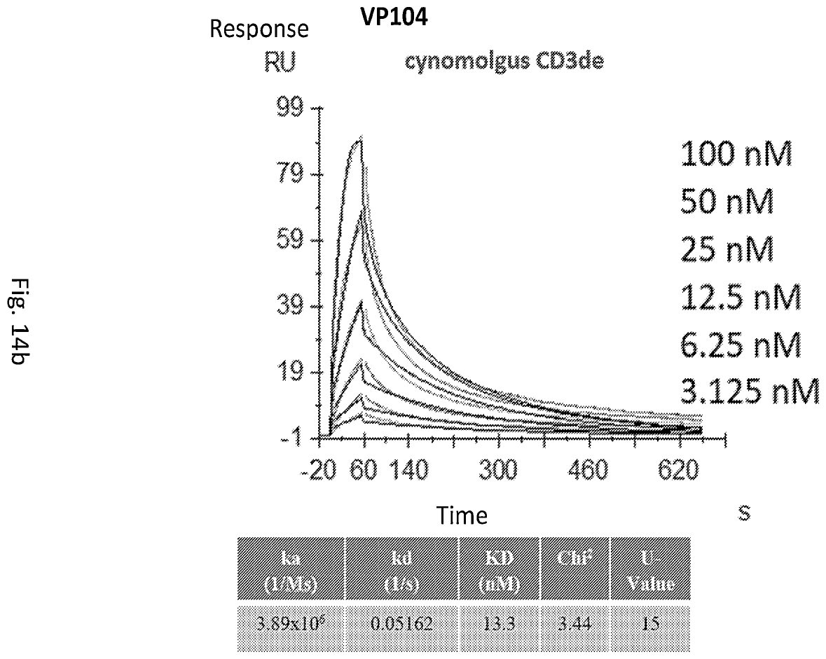

The binding affinities of VP103 (chimeric alpaca anti-CD3 VHH F10-human IgG4) and VP104 (Humanized F10A VHH (C55A)-IgG4) for recombinant human CD3de (delta epsilon subunits) and recombinant cynomolgus CD3de (delta epsilon subunits) were assessed by SPR on a Biacore T200 (GE Healthcare). FIGS. 13a and 13b show the SPR sensorgrams for VP103 to human CD3de and cynomolgus CD3de immobilized onto the surface of CM5 sensor chip. FIGS. 14a and 14b show the SPR sensorgrams for VP104 to human CD3de and cynomolgus CD3de immobilized onto the surface of CM5 sensor chip. The data were fit with a 1:1 fitting model. The KD of VP103 was 3.7 nM for human CD3de and 2.8 nM for cynomolgus CD3de. The KD of VP103 was 14.2 nM for human CD3de and 13.3 nM for cynomolgus CD3de. It is expected that the KD of the huF10 with free Cys found in VP105 would have KD values between what was observed for VP103 and VP104. The close correspondence of the affinities for the human CD3de and cynomolgus CD3de of VP103 and VP104 make these constructs highly useful for running pre-clinical toxicology studies in cynomolgus monkeys. These constructs can also be paired with anti-CD25 VP008, which is based on basiliximab, which is known to cross-react with cynomolgus CD25.

### Example 15: In Vitro Study to Assess Treg Activation in a Gut Mucosal-Like Environment

In a separate in vitro study, T cells from 3 PBMC donors were isolated and expanded using Dynabeads™ Human T-Expander CD3/CD28 (ThermoFisher Scientific) at a 3:1 bead-to-cell ratio and treated with 30 U/mL IL-2 and 1 μM retinoic acid, a chemical known to induce upregulation of gut homing receptors and mucosal-like phenotype in tissue culture (https://pubmed.ncbi.nlm.nih.gov/25027601/, https://pubmed.ncbi.nlm.nih.gov/20944006/). The expanded T-cells were then treated with 5 test articles (VP008, VP021, VP022, VP100, VP100, VP101) or Teplizumab biosimilar or media alone and a physiological concentration of IL-2 (0.3 U/mL) for 72 hours. The test articles were administered at either 1 μg/mL or 10 μg/mL. The following markers were stained for by flow cytometry at Baseline (Day 0—no treatment), 24 h and 72 h post treatment: CD3, CD4, CD8, CD25, CD127, FOXP3, LAP, Neuropilin and IL-10.

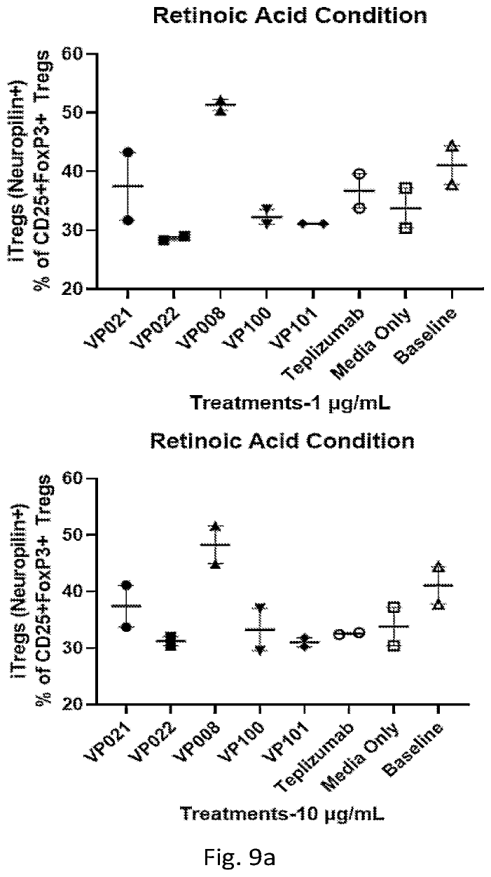

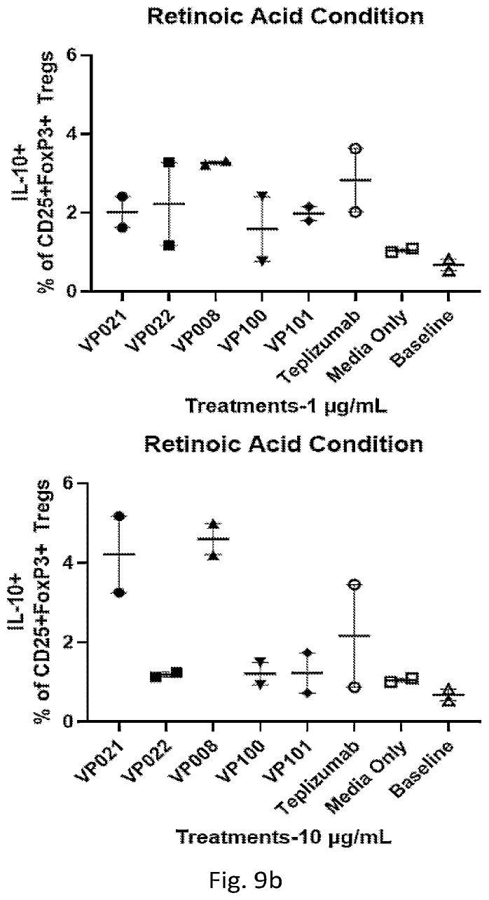

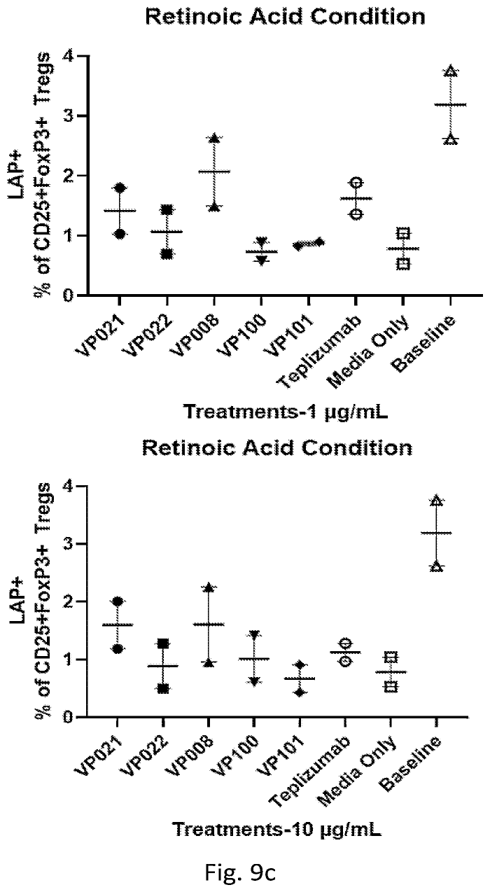

FIGS. 9a, 9b and 9c show the % Treg cells (defined as CD25+ FOXP3+) that expressed the 3 key activation markers at 24 hours of treatment: neuropilin (an induced Treg marker), IL-10, and LAP (precursor for TGFbeta). At both 1 μg/mL and 10 μg/mL, VP021 and VP008 had substantial increases in neuropilin compared to Teplizumab and media only control (FIG. 9a). At 10 μg/mL, VP021 and VP008 had increases in IL-10 compared to Teplizumab and media only control (FIG. 9b). At 10 μg/mL, VP021 and VP008 had small increases in LAP compared to Teplizumab and media only control (FIG. 9c).

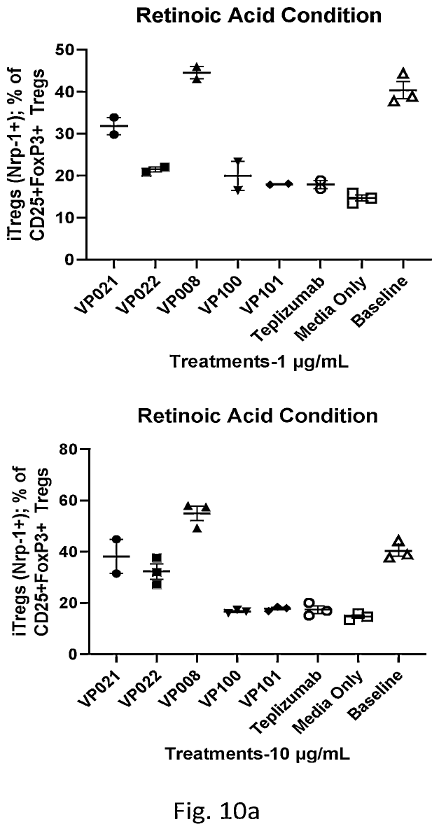

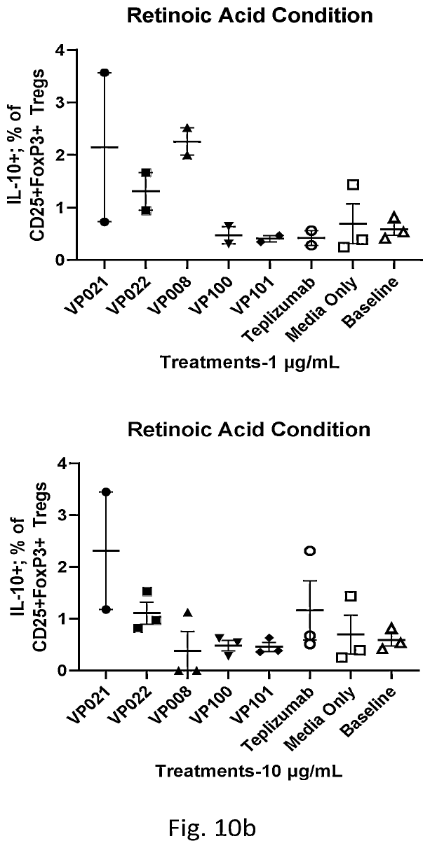

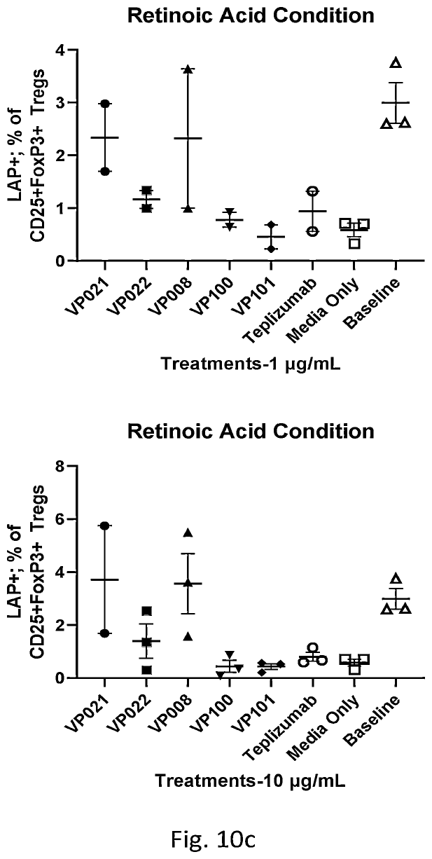

FIGS. 10a, 10b and 10c show the % Treg cells (defined as CD25+ FOXP3+) that expressed the 3 key activation markers (neuropilin, IL-10 and LAP) at 72 hours of treatment. At both 1 μg/mL and 10 μg/mL, VP021 and VP008 had substantial increases in neuropilin compared to Teplizumab and media only control (FIG. 10a). At 1 μg/mL, VP021 and VP008 had increases in IL-10 compared to Teplizumab and media only control (FIG. 10b). At 1 and 10 μg/mL, VP021 and VP008 had increases in LAP compared to Teplizumab and media only control (FIG. 10c).

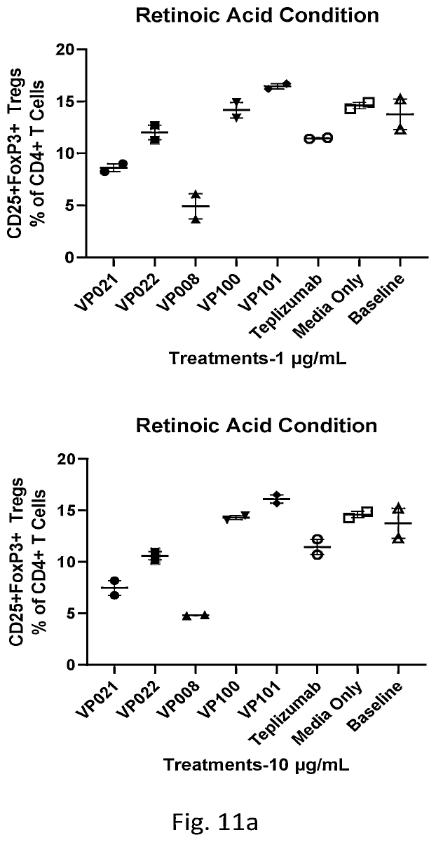

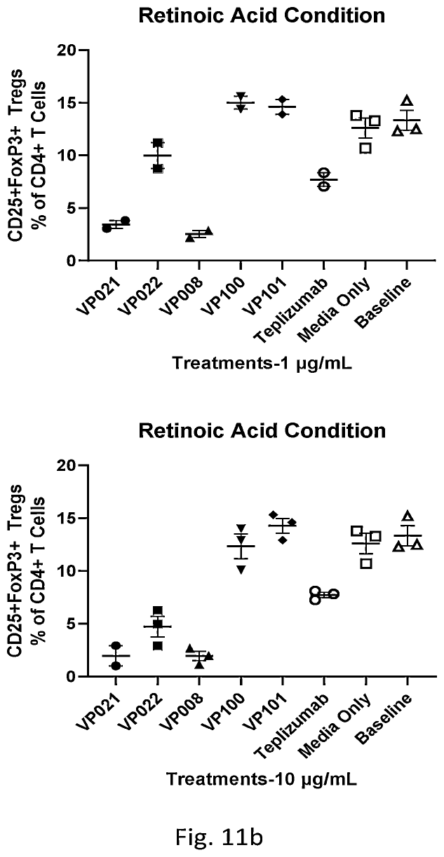

The percentage of Tregs (defined as CD25+ FOXP3+) out of the total CD4+ T cells at 24 h is shown in FIG. 11a and at 72 h is shown in FIG. 11b. Both VP021 and VP08 had lower percentages of Tregs than Teplizumab and media alone. VP100 and VP101 had highest proportion of Tregs under most conditions.

### Example 16: Depression and Experimental Autoimmune Encephalomyelitis Animal Model Using huCD3e×huCD25 Transgenic Mice

An animal experiment was designed to investigate whether orally administered anti-CD3/anti-CD25 antibodies can affect depressive behavior and Experimental Autoimmune Encephalomyelitis (EAE) in a huCD3e×huCD25 transgenic mouse model. Animals will be inoculated with MOG35-55 (EAE animals) or PBS (sham group n=4) and pertussis toxin (i.p) to induce EAE.

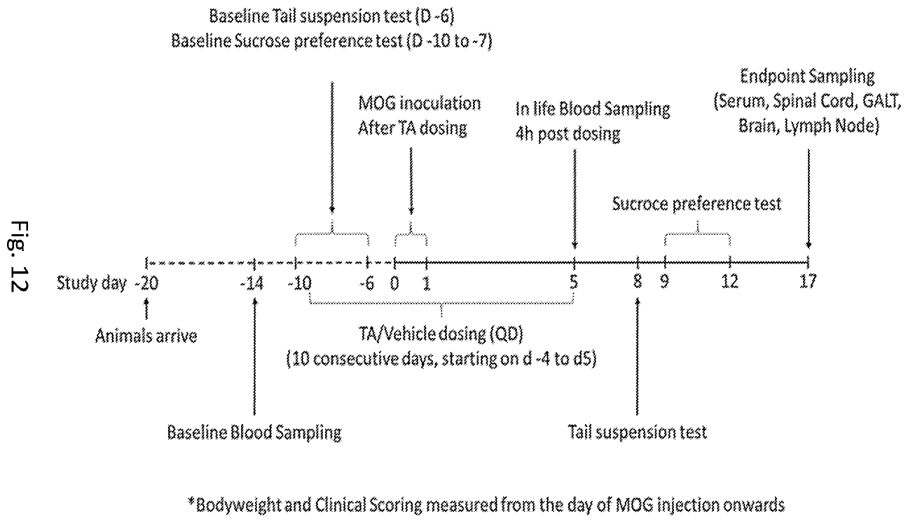

The experimental plan is shown in FIG. 12. On day −4 through day 5 animals will be dosed orally (p.o) with vehicle or test compounds (VP008 or VP021). All mice will be subjected to daily body weight measurement and daily clinical scoring throughout the study. The mice will be subjected to tail suspension test at baseline (d-6) and on day 8, and a sucrose preference test at baseline (Day −10 to −7 before MOG implantation) and on days 9 to 12. Blood sampling and serum collection will be performed at baseline (14 days before MOG implantation) and on days 5 (4 h post dosing) and on d17 end point collection. In the end point the animals will be euthanized and Spinal cord, Gut-associated lymphoid tissues (GALT) and brain collected and immuno-profiled.

1-206. (canceled)

207. An antibody or fragment thereof, capable of binding to CD25, wherein said antibody or fragment thereof is humanized and comprises either:
variable light chain CDR sequences of SEQ ID NO: 125, 126 and 127 and variable heavy chain CDR sequences of SEQ ID NO: 128, 129 and 130, or
variable light chain CDR sequences of SEQ ID NO: 79, 80 and 81 and heavy chain CDR sequences of SEQ ID NO: 82, 83 and 84.

208. The antibody or fragment thereof according to claim 207, wherein said antibody or fragment thereof comprises a VL sequence selected from SEQ ID NO: 32, 33, 34 and 120 and a VH sequence selected from SEQ ID NO: 29, 30, 31 and 121.

209. The antibody or fragment thereof according to claim 207, being a bispecific or multispecific antibody that is additionally capable of binding to CD3.

210. The antibody or fragment thereof according to claim 209, wherein said antibody or fragment thereof comprises one or both of variable light chain CDR sequences of SEQ ID NO: 73, 74 and 75 and variable heavy chain CDR sequences of SEQ ID NO: 76, 77 and 78.

211. The antibody or fragment thereof according to claim 209, wherein said antibody or fragment thereof comprises a variable heavy chain domain selected from SEQ ID NO: 6-10 and a variable light chain domain selected from SEQ ID NO: 1-5.

212. The antibody or fragment thereof according to claim 209, wherein said antibody or fragment thereof comprises the CDR sequences SEQ ID NO: 131, 132 and 133 of an alpaca VHH nanobody F10.

213. The antibody or fragment thereof according to claim 212, wherein said antibody or fragment thereof comprises a sequence selected from SEQ ID NO: 123 and SEQ ID NO: 124.

214. The antibody or fragment thereof according to claim 209, wherein said antibody or fragment thereof comprises a scFv, wherein said scFv binds CD3 and comprises a sequence selected from SEQ ID NO: 11 through SEQ ID NO: 28.

215. The antibody or fragment thereof according to claim 207, wherein said antibody or fragment thereof is a heterodimeric antibody comprising one or more of IgG1, IgG2 and IgG4 constant domain(s).

216. The antibody according to claim 215, wherein said heterodimeric antibody comprises a sequence selected from SEQ ID NO: 51 through SEQ ID NO: 69.

217. A pharmaceutical composition comprising an antibody or fragment thereof according to claim 207, and an excipient, such as a pharmaceutically-acceptable carrier.

218. An isolated nucleic acid molecule encoding an antibody agent or fragment thereof according to claim 207.

219. A recombinant vector comprising the nucleic acid molecule of claim 218.

220. A host cell comprising the recombinant vector of claim 219.

221. A method of preparing an antibody or fragment thereof according to claim 207, comprising the steps of:
providing a host cell of claim 220;
cultivating the host cell under conditions leading to expression of the antibody or fragment thereof; and
recovering the antibody or fragment thereof.

222. A method of preventing, treating and/or alleviating of one or more of an inflammatory disease, a psycho-immune disorder, an auto-immune disease and a mood disorder, the method comprising administering to a patient in need thereof a therapeutic amount of an antibody or fragment thereof capable of binding to one or both of CD3 and CD25.

223. The method of claim 222, wherein the antibody or fragment thereof capable of binding to one or both of CD3 and CD25 is an antibody or fragment thereof according to claim 207.

224. The method of claim 222, wherein said inflammatory disease, psycho-immune disorder, auto-immune disease or mood disorder is selected from the group consisting of depression, major depressive disorder, autism (ASD) and attention deficit/hyperactivity disorder (ADHD), bipolar disorder, seasonal affective disorder (SAD), cyclothymic disorder, premenstrual dysphoric disorder, persistent depressive disorder, disruptive mood dysregulation disorder, depression related to medical illness, depression induced by substance use or medication, Alzheimer's disease, Parkinson's disease and Multiple sclerosis.

225. The method of claim 222, wherein the patient is an adult or a pediatric patient.

226. The method of claim 222, wherein said antibody of fragment thereof is administered orally and/or nasally.


SEQUENCE LISTING (134 sequences)

SEQ ID NO: 1 - protein, 106 aa, Mus musculus [sequence omitted]

SEQ ID NO: 2 - protein, 106 aa, Artificial Sequence [sequence omitted]

SEQ ID NO: 3 - protein, 106 aa, Artificial Sequence
  VCD3-L1
DIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 4 - protein, 106 aa, Artificial Sequence
  VCD3-L2
EIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 5 - protein, 106 aa, Artificial Sequence
  VCD3-L3
EIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 6 - protein, 119 aa, Mus musculus [sequence omitted]

SEQ ID NO: 7 - protein, 119 aa, Artificial Sequence [sequence omitted]

SEQ ID NO: 8 - protein, 119 aa, Artificial Sequence
  VCD3-H1
QVQLVQSGGGVVQPGRSLRLSCAASGYTFTRYTMHWVRQAPGKGLEWVGYINPSRGYTNYTDSVKGRFTISTDKSKNTAYLQMNSLRAEDTAVYYCARYYDDHYSLDYWGQGTTVTVSS

SEQ ID NO: 9 - protein, 119 aa, Artificial Sequence
  VCD3-H2
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSS

SEQ ID NO: 10 - protein, 119 aa, Artificial Sequence
  VCD3-H3
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSS

SEQ ID NO: 11 - protein, 255 aa, Artificial Sequence
  VCD3-H1L1
QVQLVQSGGGVVQPGRSLRLSCAASGYTFTRYTMHWVRQAPGKGLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 12 - protein, 255 aa, Artificial Sequence
  VCD3-H1L1-DS
QVQLVQSGGGVVQPGRSLRLSCAASGYTFTRYTMHWVRQAPGKCLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGCGTKLEIK

SEQ ID NO: 13 - protein, 255 aa, Artificial Sequence
  VCD3-H1L2
QVQLVQSGGGVVQPGRSLRLSCAASGYTFTRYTMHWVRQAPGKGLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 14 - protein, 255 aa, Artificial Sequence
  VCD3-H1L2-DS
QVQLVQSGGGVVQPGRSLRLSCAASGYTFTRYTMHWVRQAPGKCLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGCGTKLEIK

SEQ ID NO: 15 - protein, 255 aa, Artificial Sequence
  VCD3-H1L3
QVQLVQSGGGVVQPGRSLRLSCAASGYTFTRYTMHWVRQAPGKGLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 16 - protein, 255 aa, Artificial Sequence
  VCD3-H1L3-DS
QVQLVQSGGGVVQPGRSLRLSCAASGYTFTRYTMHWVRQAPGKCLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIK

SEQ ID NO: 17 - protein, 255 aa, Artificial Sequence
  VCD3-H2L1
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 18 - protein, 255 aa, Artificial Sequence
  VCD3-H2L1-DS
QVQLVQSGAEVKKPGASVKVSCKASGYTFTRYTMHWVRQAPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGCGTKLEIK

SEQ ID NO: 19 - protein, 255 aa, Artificial Sequence
  VCD3-H2L2
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 20 - protein, 255 aa, Artificial Sequence
  VCD3-H2L2-DS
QVQLVQSGAEVKKPGASVKVSCKASGYTFTRYTMHWVRQAPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGCGTKLEIK

SEQ ID NO: 21 - protein, 255 aa, Artificial Sequence
  VCD3-H2L3
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 22 - protein, 255 aa, Artificial Sequence
  VCD3-H2L3-DS
QVQLVQSGAEVKKPGASVKVSCKASGYTFTRYTMHWVRQAPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIK

SEQ ID NO: 23 - protein, 255 aa, Artificial Sequence
  VCD3-H3L1
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 24 - protein, 255 aa, Artificial Sequence
  VCD3-H3L1-DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGCGTKLEIK

SEQ ID NO: 25 - protein, 255 aa, Artificial Sequence
  VCD3-H3L2
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 26 - protein, 255 aa, Artificial Sequence
  VCD3-H3L2-DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGCGTKLEIK

SEQ ID NO: 27 - protein, 255 aa, Artificial Sequence
  VCD3-H3L3
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 28 - protein, 255 aa, Artificial Sequence
  VCD3-H3L3-DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIK

SEQ ID NO: 29 - protein, 117 aa, Artificial Sequence [sequence omitted]

SEQ ID NO: 30 - protein, 117 aa, Artificial Sequence
  VCD25-H1
QVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQGTTVTVSS

SEQ ID NO: 31 - protein, 117 aa, Artificial Sequence
  VCD25-H2
QVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQGTTVTVSS

SEQ ID NO: 32 - protein, 104 aa, Artificial Sequence [sequence omitted]

SEQ ID NO: 33 - protein, 104 aa, Artificial Sequence
  VCD25-L1
QIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGQGTKLEIK

SEQ ID NO: 34 - protein, 104 aa, Artificial Sequence
  VCD25-L2
EIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGQGTKLEIK

SEQ ID NO: 35 - protein, 251 aa, Artificial Sequence
  VCD25-H1L1
QVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSQIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGQGTKLEIK

SEQ ID NO: 36 - protein, 251 aa, Artificial Sequence
  VCD25-H1L1-DS
QVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQCTTVTVSSGGGGSGGGGSGGGGSGGGGSGGGGSGGGGSQIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGCGTKLEIK

SEQ ID NO: 37 - protein, 251 aa, Artificial Sequence
  VCD25-H1L2
QVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGQGTKLEIK

SEQ ID NO: 38 - protein, 251 aa, Artificial Sequence
  VCD25-H1L2-DS
QVQLVQSGAGVAKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIK

SEQ ID NO: 39 - protein, 251 aa, Artificial Sequence
  VCD25-H2L1
QVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSQIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGQGTKLEIK

SEQ ID NO: 40 - protein, 251 aa, Artificial Sequence
  VCD25-H2L1-DS
QVQLVQSGAEVKKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSQIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGCGTKLEIK

SEQ ID NO: 41 - protein, 251 aa, Artificial Sequence
  VCD25-H2L2
QVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGQGTKLEIK

SEQ ID NO: 42 - protein, 251 aa, Artificial Sequence
  VCD25-H2L2-DS
QVQLVQSGAEVKKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIK

SEQ ID NO: 43 - protein, 251 aa, Artificial Sequence
  VCD25-L1H1
QIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGQGTKLEIKGGGGSGGGGSGGGGSGGGGSGGGGSGGGGSQVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQGTTVTVSS

SEQ ID NO: 44 - protein, 251 aa, Artificial Sequence
  VCD25-L1H1-DS
QIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGCGTKLEIKGGGGSGGGGSGGGGSGGGGSGGGGSGGGGSQVQLVQSGAGVAKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSS

SEQ ID NO: 45 - protein, 251 aa, Artificial Sequence
  VCD25-L1H2
QIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGQGTKLEIKGGGGSGGGGSGGGGSGGGGSGGGGSGGGGSQVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQGTTVTVSS

SEQ ID NO: 46 - protein, 251 aa, Artificial Sequence
  VCD25-L1H2-DS
QIVSTQSPDTQSVTPKEKVTITCRASSSRSYMQWYQQKPDQSPKRWIYDTSKSASGVPSRFSGSGSGTDYTLTINSLEAEDAATYYCHQRSSYTFGCGTKLEIKGGGGSGGGGSGGGGSGGGGSGGGGSGGGGSQVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQCTTVTVSS

SEQ ID NO: 47 - protein, 251 aa, Artificial Sequence
  VCD25-L2H1
EIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGQGTKLEIKGGGGSGGGGSGGGGSGGGGSGGGGSGGGGSQVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQGTTVTVSS

SEQ ID NO: 48 - protein, 251 aa, Artificial Sequence
  VCD25-L2H1-DS
EIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIKGGGGSGGGGSGGGGSGGGGSGGGGSGGGGSQVQLVQSGAGVAKPGASVKVSCKASGYSFTRYWMHWVRQPPGQGLEWMGAIYPGNSDTSYSQKFEGRVTITADTSASTAYMELSSLRSEDTAVYYCSRDYGYYFDFWGQCTTVTVSS

SEQ ID NO: 49 - protein, 251 aa, Artificial Sequence
  VCD25-L2H2
EIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGQGTKLEIKGGGGSGGGGSGGGGSGGGGSGGGGSGGGGSQVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQGTTVTVSS

SEQ ID NO: 50 - protein, 251 aa, Artificial Sequence
  VCD25-L2H2-DS
EIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIKGGGGSGGGGSGGGGSGGGGSGGGGSGGGGSQVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQCTTVTVSS

SEQ ID NO: 51 - protein, 330 aa, Homo Sapiens [sequence omitted]

SEQ ID NO: 52 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 327 aa, Artificial Sequence
  V-IGG4-A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 242 aa, Artificial Sequence
  V-IGG4-A-Fc
GGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 327 aa, Artificial Sequence
  V-IGG4-B
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 56 - protein, 242 aa, Artificial Sequence
  V-IGG4-B-Fc
GGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 57 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 58 - protein, 327 aa, Artificial Sequence
  V-IGG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 59 - protein, 241 aa, Artificial Sequence
  V-IGG2-Fc
GGGGSGGGGSGGGGSDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 60 - protein, 327 aa, Artificial Sequence
  V-IGG2-A
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 61 - protein, 241 aa, Artificial Sequence
  V-IGG2-A-Fc
GGGGSGGGGSGGGGSDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 62 - protein, 327 aa, Artificial Sequence
  V-IGG2-B
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 63 - protein, 241 aa, Artificial Sequence
  V-IGG2-B-Fc
GGGGSGGGGSGGGGSDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 64 - protein, 326 aa, Artificial Sequence
  V-IGG2-C
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 65 - protein, 240 aa, Artificial Sequence
  V-IGG2-C-Fc
GGGGSGGGGSGGGGSYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 66 - protein, 326 aa, Artificial Sequence
  V-IGG2-D
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 67 - protein, 240 aa, Artificial Sequence
  V-IGG2-D-Fc
GGGGSGGGGSGGGGSYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 68 - protein, 326 aa, Artificial Sequence
  V-IGG2-E
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 69 - protein, 240 aa, Artificial Sequence
  V-IGG2-E-Fc
GGGGSGGGGSGGGGSYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 70 - protein, 107 aa, Homo Sapiens [sequence omitted]

SEQ ID NO: 71 - protein, 5 aa, Artificial Sequence
  Linker sequence
GGGGS

SEQ ID NO: 72 - protein, 7 aa, Artificial Sequence
  Hinge sequence
CPPCPAP

SEQ ID NO: 73 - protein, 5 aa, Artificial Sequence
  CD3 VL CDR1
SSVSY

SEQ ID NO: 74 - protein, 3 aa, Artificial Sequence
  CD3 VL CDR2
DTS

SEQ ID NO: 75 - protein, 9 aa, Artificial Sequence
  CD3 VL CDR3
QQWSSNPFT

SEQ ID NO: 76 - protein, 8 aa, Artificial Sequence
  CD3 VH CDR1
GYTFTRYT

SEQ ID NO: 77 - protein, 8 aa, Artificial Sequence
  CD3 VH CDR2
INPSRGYT

SEQ ID NO: 78 - protein, 12 aa, Artificial Sequence
  CD3 VH CDR3
ARYYDDHYCLDY

SEQ ID NO: 79 - protein, 5 aa, Artificial Sequence
  CD25 VL CDR1
SSRSY

SEQ ID NO: 80 - protein, 3 aa, Artificial Sequence
  CD25 VL CDR2
DTS

SEQ ID NO: 81 - protein, 7 aa, Artificial Sequence
  CD25 VL CDR3
HQRSSYT

SEQ ID NO: 82 - protein, 8 aa, Artificial Sequence
  CD25 VH CDR1
GYSFTRYW

SEQ ID NO: 83 - protein, 8 aa, Artificial Sequence
  CD25 VH CDR2
IYPGNSDT

SEQ ID NO: 84 - protein, 10 aa, Artificial Sequence
  CD25 VH CDR3
SRDYGYYFDF

SEQ ID NO: 85 - protein, 11 aa, Homo Sapiens
CCVECPPCPAP

SEQ ID NO: 86 - protein, 12 aa, Homo Sapiens
DKTHTCPPCPAP

SEQ ID NO: 87 - protein, 11 aa, Artificial Sequence
  IgG4 hinge with S228P
YGPPCPPCPAP

SEQ ID NO: 88 - protein, 107 aa, Homo Sapiens [sequence omitted]

SEQ ID NO: 89 - protein, 106 aa, Homo Sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 90 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 91 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 92 - protein, 106 aa, Homo Sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 93 - protein, 106 aa, Homo Sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 94 - protein, 213 aa, Artificial
  VP001 Tep-Vl with KCl
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 446 aa, Artificial
  VP001 Tep-VH C114S with V-IGG4-A
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 659 aa, Artificial
  VP002 VCD3-L3 with KCL
EIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 479 aa, Artificial
  VP003 VIT-106 LC VCD3-L3 - KCL- linker- VCD25-H2L2-DS
EIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGAEVKKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIK

SEQ ID NO: 98 - protein, 327 aa, Artificial; identical to SEQ ID NO: 53
  VP003 VCD3-H3 with V-IGG4-A

SEQ ID NO: 99 - protein, 497 aa, Artificial
  VP004 Tep-VH-VL scFv C114S DS with V-IGG4-A-Fc
QVQLVQSGGGVVQPGRSLRLSCKASGYTFTRYTMHWVRQAPGKCLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 497 aa, Artificial
  VP005 VCD3-H3L3-DS with V-IGG4-A-Fc
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIKGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 763 aa, Artificial
  VP006 VIT-109 VCD3-H3L3-VIGG4-A-Fc with VCD25 H2L2 DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIKGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVQLVQSGAEVKKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIK

SEQ ID NO: 102 - protein, 211 aa, Artificial
  VP007 mBasil VL with KCL
QIVSTQSPAIMSASPGEKVTMTCSASSSRSYMQWYQQKPGTSPKRWIYDTSKLASGVPARFSGSGSGTSYSLTISSMEAEDAATYYCHQRSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 444 aa, Artificial
  VP007 mBasil VH with V-IGG4-B
QVQLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 104 - protein, 211 aa, Artificial
  VP008 VCD25-L2 with KCL
EIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 444 aa, Artificial
  VP008 VCD25-H2 with V-IGG4-B
QVQLVQSGAEVKKPGASVKVSCKASGYSFTRYWMHWVRQPPGQRLEWMGAIYPGNSDTSYSQKFEGRVTITADTSASTAYMELSSLRSEDTAVYYCSRDYGYYFDFWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 106 - protein, 493 aa, Artificial
  VP009 Bas VH-VL with DS with V-IGG4-B-Fc
QVQLVQSGAEVKKPGASVKVSCKASGYSFTRYWMHWVRQPPGQCLEWMGAIYPGNSDTSYSQKFEGRVTITADTSASTAYMELSSLRSEDTAVYYCSRDYGYYFDFWGQGTTVTVSSGGGGSGGGGSGGGGSGGGGSGGGGSGGGGSEIVLTQSPDFQSVTPKEKVTITCRASSSRSYMQWYQQKPDQSPKRLIYDTSKSASGVPSRFSGSGSGTDYTLTINSLEAEDAATYYCHQRSSYTFGCGTKLEIKGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 107 - protein, 493 aa, Artificial; identical to SEQ ID NO: 106
  VP010 VCD25-H2L2-DS with V-IGG4-B-Fc

SEQ ID NO: 108 - protein, 213 aa, Artificial
  VP100 ch7G7 VL with KCL
QIVLSQSPAILSASPGERVTMTCRASSSVSFMHWLQQKPGSSPKPWIYATSNLASGVSARFSGSGSGTSYSLTITRVEAEDAATYYCQQWSSNPPAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 450 aa, Artificial
  VP100 ch7G7 with V-IGG4-B-Fc
EVQLVESGGDLVQPRGSLKLSCAASGFTFSSYGMSWVRQTPDKRLELVATINGYGDTTYYPDSVKGRFTISRDNAKNTLYLQMSSLKSEDTAMYFCARDRDYGNSYYYALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 110 - protein, 213 aa, Artificial
  VP101 hu7G7 VL with KCL
EIVLTQSPATLSLSPGERATLSCRASSSVSFMHWLQQKPGQAPKPLIYATSNLASGIPARFSGSGSGTDYTLTISSLEPEDFAVYYCQQWSSNPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 450 aa, Artificial
  VP101 hu7G7 with V-IGG4-B-Fc
EVQLVESGGGLVQPGGSLRLSCAASGFTFSSYGMSWVRQAPGKRLELVATINGYGDTTYYPDSVKGRFTISRDNAKNTLYLQMNSLRAEDTAVYYCARDRDYGNSYYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 112 - protein, 771 aa, Artificial
  VP102A Tep-VH-VL scFv C114S DS and hu7G7 scFv with V-IGG4-A-Fc
QVQLVQSGGGVVQPGRSLRLSCKASGYTFTRYTMHWVRQAPGKCLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITGGGGSGGGGSGGGGSEVQLVESGGGLVQPGGSLRLSCAASGFTFSSYGMSWVRQAPGKCLELVATINGYGDTTYYPDSVKGRFTISRDNAKNTLYLQMNSLRAEDTAVYYCARDRDYGNSYYYALDYWGQGTLVTVSSGGGGSGGGGSGGGGSGGGGSGGGGSGGGGSEIVLTQSPATLSLSPGERATLSCRASSSVSFMHWLQQKPGQAPKPLIYATSNLASGIPARFSGSGSGTDYTLTISSLEPEDFAVYYCQQWSSNPPAFGCGTKVEIKGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 771 aa, Artificial
  VP102B Tep-VH-VL scFv C114S DS with V-IGG4-A-Fc and and hu7G7 scFv
QVQLVQSGGGVVQPGRSLRLSCKASGYTFTRYTMHWVRQAPGKCLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGFTFSSYGMSWVRQAPGKCLELVATINGYGDTTYYPDSVKGRFTISRDNAKNTLYLQMNSLRAEDTAVYYCARDRDYGNSYYYALDYWGQGTLVTVSSGGGGSGGGGSGGGGSGGGGSGGGGSGGGGSEIVLTQSPATLSLSPGERATLSCRASSSVSFMHWLQQKPGQAPKPLIYATSNLASGIPARFSGSGSGTDYTLTISSLEPEDFAVYYCQQWSSNPPAFGCGTKVEIK

SEQ ID NO: 114 - protein, 365 aa, Artificial
  VP103 Alpaca Anti-CD3 nanobody F10 with with V-IGG4-A-Fc
QVQLQESGGGLVQAGGSLTLSCAASGFTLDDYAIGWFRQAPGKEREGVSCIRRSDGHTYSADSVKGRFTISSDNAKNTVYLQMNNLKPEDTAVYYCAAGTGWGRYFYAGMDYWGKGTQVTVSSGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 365 aa, Artificial
  VP104 humanized Anti-CD3 nanobody F10 with free Cys removed and with V-IGG4-A-Fc
EVQLLESGGGLVQPGGSLRLSCAASGFTLDDYAIGWVRQAPGKGREGVSAIRRSDGHTYYADSVKGRFTISSDNSKNTVYLQMNSLRAEDTAVYYCAAGTGWGRYFYAGMDYWGQGTLVTVSSGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 365 aa, Artificial
  VP105 humanized Anti-CD3 nanobody F10 (contians free Cys) with V-IGG4-A-Fc
EVQLLESGGGLVQPGGSLRLSCAASGFTLDDYAIGWVRQAPGKGREGVSCIRRSDGHTYYADSVKGRFTISSDNSKNTVYLQMNSLRAEDTAVYYCAAGTGWGRYFYAGMDYWGQGTLVTVSSGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 639 aa, Artificial
  VP106 humanized Anti-CD3 nanobody F10 (contians free Cys) and hu7G7 scFv with V-IGG4-A-Fc
EVQLLESGGGLVQPGGSLRLSCAASGFTLDDYAIGWVRQAPGKGREGVSAIRRSDGHTYYADSVKGRFTISSDNSKNTVYLQMNSLRAEDTAVYYCAAGTGWGRYFYAGMDYWGQGTLVTVSSGGGGSGGGGSGGGGSEVQLVESGGGLVQPGGSLRLSCAASGFTFSSYGMSWVRQAPGKCLELVATINGYGDTTYYPDSVKGRFTISRDNAKNTLYLQMNSLRAEDTAVYYCARDRDYGNSYYYALDYWGQGTLVTVSSGGGGSGGGGSGGGGSGGGGSGGGGSGGGGSEIVLTQSPATLSLSPGERATLSCRASSSVSFMHWLQQKPGQAPKPLIYATSNLASGIPARFSGSGSGTDYTLTISSLEPEDFAVYYCQQWSSNPPAFGCGTKVEIKGGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 106 aa, Artificial
  m7G7-VL
QIVLSQSPAILSASPGERVTMTCRASSSVSFMHWLQQKPGSSPKPWIYATSNLASGVSARFSGSGSGTSYSLTITRVEAEDAATYYCQQWSSNPPAFGGGTKLEIK

SEQ ID NO: 119 - protein, 123 aa, Artificial
  m7G7-VH
EVQLVESGGDLVQPRGSLKLSCAASGFTFSSYGMSWVRQTPDKRLELVATINGYGDTTYYPDSVKGRFTISRDNAKNTLYLQMSSLKSEDTAMYFCARDRDYGNSYYYALDYWGQGTSVTVSS

SEQ ID NO: 120 - protein, 106 aa, Artificial
  hu7G7-VL
EIVLTQSPATLSLSPGERATLSCRASSSVSFMHWLQQKPGQAPKPLIYATSNLASGIPARFSGSGSGTDYTLTISSLEPEDFAVYYCQQWSSNPPAFGGGTKVEIK

SEQ ID NO: 121 - protein, 123 aa, Artificial
  hu7G7-VH
EVQLVESGGGLVQPGGSLRLSCAASGFTFSSYGMSWVRQAPGKRLELVATINGYGDTTYYPDSVKGRFTISRDNAKNTLYLQMNSLRAEDTAVYYCARDRDYGNSYYYALDYWGQGTLVTVSS

SEQ ID NO: 122 - protein, 123 aa, Artificial
  Alpaca F10 VHH
QVQLQESGGGLVQAGGSLTLSCAASGFTLDDYAIGWFRQAPGKEREGVSCIRRSDGHTYSADSVKGRFTISSDNAKNTVYLQMNNLKPEDTAVYYCAAGTGWGRYFYAGMDYWGKGTQVTVSS

SEQ ID NO: 123 - protein, 123 aa, Artificial
  Humanized F10VHH
EVQLLESGGGLVQPGGSLRLSCAASGFTLDDYAIGWVRQAPGKGREGVSCIRRSDGHTYYADSVKGRFTISSDNSKNTVYLQMNSLRAEDTAVYYCAAGTGWGRYFYAGMDYWGQGTLVTVSS

SEQ ID NO: 124 - protein, 123 aa, Artificial
  Humanized F10A VHH
EVQLLESGGGLVQPGGSLRLSCAASGFTLDDYAIGWVRQAPGKGREGVSAIRRSDGHTYYADSVKGRFTISSDNSKNTVYLQMNSLRAEDTAVYYCAAGTGWGRYFYAGMDYWGQGTLVTVSS

SEQ ID NO: 125 - protein, 5 aa, Artificial
  7G7 VL-CDR1
SSVSF

SEQ ID NO: 126 - protein, 3 aa, Artificial
  7G7 VL-CDR2
ATS

SEQ ID NO: 127 - protein, 9 aa, Artificial
  7G7-VL-CDR3
QQWSSNPPA

SEQ ID NO: 128 - protein, 8 aa, Artificial
  7G7-VH-CDR1
GFTFSSYG

SEQ ID NO: 129 - protein, 8 aa, Artificial
  7G7-VH_CDR2
INGYGDTT

SEQ ID NO: 130 - protein, 16 aa, Artificial
  7G7-VH-CDR3
ARDRDYGNSYYYALDY

SEQ ID NO: 131 - protein, 8 aa, Artificial
  F10 VHH-CDR1
GFTLDDYA

SEQ ID NO: 132 - protein, 8 aa, Artificial
  F10 VHH-CDR2
IRRSDGHT

SEQ ID NO: 133 - protein, 16 aa, Artificial
  F10 VHH-CDR3
AAGTGWGRYFYAGMDY

SEQ ID NO: 134 - protein, 27 aa, Homo sapiens
TPLGDTTHTPEPKSCDTPPPCPRCPAP